(19) United States
(12) Patent Application Publication
(10) Pub. No.: US 2025/0281084 A1
(43) Pub. Date: Sep. 11, 2025

(54) COMPOSITION, COATING, AND METHOD FOR REDUCING EARLY/LATE SENSOR ATTENUATION OF ANALYTE BIOSENSOR

(71) Applicant: Abbott Diabetes Care Inc., Alameda, CA (US)

(72) Inventors: Zenghe Liu, Alameda, CA (US); Jonathan D. McCanless, Oakland, CA (US); Benjamin J. Feldman, Berkeley, CA (US); Tianmei Ouyang, Saratoga, CA (US)

(21) Appl. No.: 19/069,685

(22) Filed: Mar. 4, 2025

Related U.S. Application Data
(60) Provisional application No. 63/561,463, filed on Mar. 5, 2024.

(51) Int. Cl. A61B 5/1486; G01N 27/40

(52) U.S. Cl. CPC A61B 5/14865; G01N 27/40; A61B 2562/24

(57) ABSTRACT

The present disclosure relates to an analyte sensor comprising a working electrode, a sensing layer disposed on at least a portion of the working electrode that comprises an analyte-responsive enzyme, and a polymer membrane overcoating at least the sensing layer. The polymer membrane comprises an antimicrobial agent disposed therein, a hydrogel coating disposed thereon, or both an antimicrobial agent disposed therein and a hydrogel coating disposed thereon. The presence of an antimicrobial agent, such as an antibiotic, a hydrogel coating, or both reduces early/late signal attenuation of the analyte sensor. The polymer membrane can further be combined with a metal-containing layer in electrochemical communication with a reference electrode, a counter electrode, and/or second working electrode.

### BACKGROUND

The detection of one or more suitable analytes within an individual in need can be critical for monitoring the condition of the individual's health as deviations from normal analyte levels can be indicative of a physiological condition. For example, monitoring glucose levels can enable people suffering from diabetes to take appropriate or suitable corrective action including administration of medicine or consumption of particular food or beverage products to avoid significant physiological harm. Other analytes can be desirable to monitor for other physiological conditions.

Analyte monitoring in an individual can be periodic or continuous over a period of time. Periodic analyte monitoring can take place by withdrawing a sample of bodily fluid, such as blood or urine, at set time intervals and analyzing the bodily fluid ex vivo. In some circumstances, periodic, ex vivo analyte monitoring can be sufficient to determine the physiological condition of many individuals. However, ex vivo analyte monitoring can be inconvenient or painful. Moreover, there is no way to recover lost data when an analyte measurement is not obtained at an appropriate or suitable time. Continuous analyte monitoring can be conducted utilizing one or more sensors that remain at least partially implanted within a tissue (e.g., skin) of an individual, such as dermally, subcutaneously, or intravenously, so that analyses can be conducted in vivo. Implanted sensors can collect analyte data on-demand, at a set schedule, or continuously, depending on an individual's particular health needs and/or previously measured analyte levels. Analyte monitoring with an in vivo implanted sensor can be a more desirable approach for individuals having severe analyte dysregulation and/or rapidly fluctuating analyte levels, although it can also be beneficial for other individuals as well.

However, in vivo (transcutaneous or implanted) sensor performance is highly dependent upon biologic events local to the sensor. Generally, for an accurate measurement, an analyte needs an undisturbed/consistent/predictable sensing pathway to communicate with the sensing elements. In electrochemical sensors, stable connectivity between sensor half cell(s) assures proper sensor function. Maintaining these pathways throughout sensor life is critical. However, implantable sensors with a long-term wear duration (e.g., 30-day wear duration) can be plagued by short life spans due to a late signal attenuation (LSA) when implanted in vivo. The in vivo loss and/or reduced sensitivity of sensor function seen in implantable sensors during a long-term wear, i.e., LSA, is thought to be caused by certain biological processes, including immune responses (e.g., foreign body response), infection, inflammation, fibrosis, and vessel regression, that occur in the tissue around (e.g., surrounding) implanted portion of the analyte sensor.

Disturbances, including chemical and physical disturbances, to sensing pathways as a result of microbes are of great concern. Microorganisms can populate the implant site (space adjacent to the implanted surface of the analyte sensor) and influence analyte concentration local to the analyte sensor by either artificially increasing or decreasing a local analyte level, and microorganisms at the sensor implant site can produce a localized environment that can affect sensor functions. For example, a dense microorganism layer can consume a portion of an analyte of interest before it reaches the analyte sensor, producing an artificially low reading when attempting to measure the interstitial fluid analyte concentration with a transdermal sensor.

In some aspects, microorganism infection at the implant site can lead to biofilm formation directly on the implant surface. Biofilms typically are dense networks of bacteria cells embedded in DNA, proteins, and polysaccharides and can hinder analyte diffusion to the sensing area, resulting in artificially low analyte readings. Biofouling from protein and other molecule adsorption onto sensor surfaces can produce an increasing and limiting diffusion barrier between a sensor and an analyte, resulting in artificially low analyte readings. Over time, a transcutaneous sensor can become walled off due to the wound healing process as the membrane dries. The reference electrode can lose connectivity with the working electrode and the sensor can malfunction.

Thus, there is a need to minimize disturbances to the sensing pathways of an in vivo analyte sensor.

### BRIEF SUMMARY

The present disclosure relates to an analyte sensor comprising
a working electrode,
a sensing layer disposed on at least a portion of the working electrode that comprises an analyte-responsive enzyme, and
a polymer membrane overcoating at least the sensing layer,
wherein the polymer membrane comprises an antimicrobial agent disposed therein, a hydrogel coating disposed thereon, or both an antimicrobial agent disposed therein and hydrogel coating disposed thereon.

The present disclosure further relates to an analyte sensor for measuring an analyte concentration in a bodily fluid of a user, the analyte sensor comprising:
a proximal (or first) portion configured to be positioned above a user's skin; and
a distal (or second) portion configured to be transcutaneously positioned beneath the skin and in contact with bodily fluid to detect the analyte in vivo,
the distal portion comprising a working electrode, a reference electrode, and a counter electrode, each connected to contacts positioned on the proximal portion, wherein the working electrode comprises at least one sensing layer;
wherein the sensing layer comprises:
an analyte-responsive enzyme; and
a polymer membrane overcoating at least the sensing layer,
wherein the polymer membrane comprises an antimicrobial agent disposed therein, a hydrogel coating disposed thereon, or both an antimicrobial agent disposed therein and hydrogel coating disposed thereon. Thus, the analyte sensor can comprise an antimicrobial agent and/or a hydrogel coating, both of which can be optionally combined with a metal-containing layer in electrochemical communication with the reference electrode, the counter electrode, a second working electrode, or any combination thereof.

In some aspects, the polymer membrane comprises a polymer matrix formed from at least one polymer selected from the group consisting of a polyvinylpyridine-based polymer, a polyvinylimidazole-based polymer, a polyacrylate, a poly(amino acid), a polyurethane, a polyether urethane, a silicone, and any combination thereof. In some aspects, the polymer matrix comprises a polyvinylpyridine-based polymer, a polyvinylimidazole-based polymer, or a combination thereof. In some aspects, the polyvinylpyridine-based polymer is an optionally substituted polyvinylpyridine-co-polystyrene polymer.

In some aspects, the polymer matrix is further formed from a crosslinker. In some aspects, the crosslinker is selected from the group consisting of polyepoxides, carbodiimide, cyanuric chloride, triglycidyl glycerol, N-hydroxysuccinimide, imidoesters, epichlorohydrin, amine-containing compounds, and any combination thereof. In some aspects, the crosslinker is a diglycidyl- or triglycidyl-functional epoxy. In some aspects, the crosslinker is selected from the group consisting of diglycidyl-PEG 200, diglycidyl-PEG 400, diglycidyl-PEG 1000, glycerol triglycidyl ether, and any combination thereof.

In some aspects, the polymer membrane comprises an antimicrobial agent. In some aspects, the antimicrobial agent is an antibiotic, an anti-fungal agent, an anti-infective agent, or any combination thereof and is not a metal or a metal salt.

In some aspects, the antimicrobial agent comprises at least one (e.g., 1, 2, 3, or 4, etc.) antibiotic. In some aspects, the antibiotic comprises a tetracycline, such as minocycline or a salt thereof. In some aspects, the minocycline salt is minocycline hydrochloride, such as about 0.1 to about 5 wt % minocycline hydrochloride, based on a total weight of the polymer membrane (dry mass). In some aspects, the antibiotic comprises the tetracycline in combination with an ansamycin. In some aspects, the ansamycin is a rifamycin, such as rifampin. In some aspects, the antibiotic comprises minocycline hydrochloride and rifampin. In some aspects, a weight ratio of minocycline hydrochloride to rifampin is in a range of about 1:10 to about 10:1. In some aspects, a weight ratio of minocycline hydrochloride to rifampin is in a range of about 1:6 to about 1:4.

In some aspects, a total amount of the antibiotic is in a range of about 0.1 wt % to about 40 wt % based on a total weight of the polymer matrix (i.e., dry mass). In some aspects, the total amount of the antibiotic is in a range of about 5 wt % to about 20 wt % based on a total weight of the polymer matrix (i.e., dry mass).

In some aspects of the analyte sensor, the polymer membrane comprises a hydrogel coating disposed on (e.g., overcoating) the polymer membrane.

In some aspects, the hydrogel coating is formed from (e.g., comprises) a polymer selected from poly(acrylic acid), poly-(α,β)-DL-aspartic acid, poly-L-glutamic acid, a salt form thereof, and a combination thereof, and a crosslinker. In some aspects, the polymer is poly(acrylic acid), a salt form thereof, or a combination thereof. In some aspects, the crosslinker comprises trimethylolpropane tris(2-methyl-1-aziridinepropionate).

In some aspects, the hydrogel coating further comprises a fluoride ion compound. In some aspects, the fluoride ion compound is at least one selected from an alkali metal fluoride, an alkaline earth metal fluoride, a transition metal fluoride, an ammonium fluoride, and any combination thereof. In some aspects, the fluoride ion compound is sodium fluoride. In some aspects, a total amount of the fluoride ion is in a range of about 1 wt % to about 30 wt % based on a total weight of the polymer matrix (i.e., dry mass).

In some aspects, the analyte sensor further comprises a metal-containing layer in electrochemical communication with the reference electrode, the counter electrode, a second working electrode, or any combination thereof. In some aspects, the metal-containing layer comprises: (i) a metal selected from the group consisting of silver, copper, zinc, gold, platinum, palladium, titanium, an alloy of any of the foregoing, and mixtures of any of the foregoing; and (ii) optionally a corresponding metal salt thereof. In some aspects, the metal is in a form of a film, a wire, or particles. In some aspects, the metal-containing layer comprises: (i) a silver film, silver wire, and/or silver particles; and (ii) a silver salt. In some aspects, the silver salt is silver chloride.

The present disclosure further relates to a method of actively releasing an antimicrobial agent in an analyte sensor, the method comprising:
inserting an analyte sensor described herein into skin of a patient; wherein a metal-containing layer is in electrochemical communication with a counter electrode, a reference electrode, a second working electrode, or any combination thereof; and
electrochemically generating an antimicrobial agent by applying an electric current to the analyte sensor.

In some aspects of the method, the antimicrobial agent is electrochemically generated metal ions. In some aspects, the antimicrobial agent is electrochemically generated silver ions. In some aspects, electrochemically generating an antimicrobial agent by applying an anodic electric current to the analyte sensor comprises applying a potential to oxidize the metal in the metal-containing layer.

Additional aspects and advantages of the disclosure will be set forth, in part, in the description that follows, and will flow from the description, or can be learned by practice of the disclosure.

It is to be understood that both the foregoing summary and the following detailed description are exemplary and explanatory only, and do not restrict the scope of the claims.

### BRIEF DESCRIPTION OF THE DRAWINGS

FIG. 1 shows a diagram of an illustrative sensing system that can incorporate an analyte sensor of the present disclosure.

FIG. 2 illustrates a cross-sectional diagram of an implantable portion of an analyte sensor according to one or more aspects of the present disclosure.

FIGS. 3A-3D illustrate a cross-sectional diagram of an implantable portion of an analyte sensor according to one or more aspects of the present disclosure.

FIG. 4 illustrates late signal attenuation of an analyte sensor caused by bacteria CFUs according to one or more aspects of the present disclosure.

FIG. 5A and FIG. 5B illustrate responses of antimicrobial sensors made with methanol-based dipping formulations and either 0 wt % (control), 6.25 wt %, 12.5 wt %, or 25 wt % antibiotic loadings according to one or more aspects of the present disclosure.

FIG. 6A illustrates responses of antimicrobial sensors made with methanol-based dipping formulations, 25 wt % antibiotic loadings, and 5-day curing according to one or more aspects of the present disclosure.

FIG. 6B illustrates responses of antimicrobial sensors made with methanol-based dipping formulations, 25 wt % antibiotic loadings, and 10-day curing according to one or more aspects of the present disclosure.

FIG. 7 illustrates responses of antimicrobial sensors made with methanol-based dipping formulations, 25 wt % antibiotic loadings, and different curing times according to one or more aspects of the present disclosure.

FIG. 8 illustrates cytotoxicity testing of antimicrobial sensors according to one or more aspects of the present disclosure.

FIG. 9 illustrates cytotoxicity testing of antimicrobial sensors including different minocycline hydrochloride loadings according to one or more aspects of the present disclosure.

FIG. 10 illustrates sensor calibration and 36-hour stability testing of antimicrobial sensors according to one or more aspects of the present disclosure.

FIG. 11 illustrates example response characteristics of antimicrobial sensors according to one or more aspects of the present disclosure.

FIG. 12 illustrates responses of analyte sensors in non-heparinized blood (82 mg/dL) in a silicone tube at 37° C. according to one or more aspects of the present disclosure. The solid line represents an analyte sensor without a hydrogel coating. The dotted line represents an analyte sensor coated with poly(acrylic acid) (PAA) hydrogel. The dashed line represents an analyte sensor coated with poly(acrylic acid) (PAA) hydrogel and sodium fluoride.

FIG. 13 illustrates responses of analyte sensors in a 30 mM glucose and 100 mM phosphate buffered saline (PBS) solution at 33° C. according to one or more aspects of the present disclosure. Black: analyte sensor without a hydrogel coating; Red: analyte sensor coated with poly(acrylic acid) hydrogel.

FIGS. 14A-14D illustrate a schematically exploded-view of an analyte sensor according to one or more aspects of the present disclosure.

FIG. 15 illustrates an electrochemical generation of AgCl according to one or more aspects of the present disclosure.

### DETAILED DESCRIPTION

The headings provided herein are not limitations of the various aspects of the disclosure, which can be defined by reference to the specification as a whole. It is also to be understood that the terminology used herein is for the purpose of describing particular aspects only, and is not intended to be limiting, since the scope of the present disclosure will be limited only by the appended claims.

### Definitions

For convenience, the meaning of some terms and phrases used in the specification, examples, and appended claims are provided below. Unless stated otherwise, or implicit from context, the following terms and phrases include the meanings provided below. The definitions are provided to aid in describing particular aspects, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification will control.

The articles “a,” “an,” and “the” are used herein to refer to one or to more than one (i.e., to at least one) of the grammatical object of the article. By way of example, “an element” means one element or more than one element.

As used herein, the terms “comprises,” “comprising,” “having,” “including,” “containing,” and the like are open-ended terms meaning “including, but not limited to.” To the extent a given aspect disclosed herein “comprises” certain elements, it should be understood that present disclosure also specifically contemplates and discloses aspects that “consist essentially of” those elements and that “consist of” those elements.

As used herein the terms “consists essentially of,” “consisting essentially of,” and the like are to be construed as a semi-closed terms, meaning that no other ingredients which materially affect the basic and novel characteristics of an aspect are included.

As used herein, the terms “consists of,” “consisting of,” and the like are to be construed as closed terms, such that an aspect “consisting of” a particular set of elements excludes any element, step, or ingredient not specified in the aspect.

Herein, “or” is not to be construed as an exclusive meaning, for example, “A or B” is construed to include A, B, A+B, and/or the like. Further, as used herein, the term “and/or” includes any and all combinations of one or more of the associated listed items. Expressions such as “at least one of,” “one of,” and “selected from,” when preceding a list of elements, modify the entire list of elements and do not modify the individual elements of the list.

As used herein, the term “about” means within an acceptable error range for the particular value as determined by one of ordinary skill in the art, which depends in part on how the value is measured or determined, i.e., the limitations of the measurement system. For example, “about” can mean within 3 or more than 3 standard deviations, per the practice in the art. Alternatively, “about” can mean a range of up to ±10% (e.g., up to ±5%, or up to ±1%) of a given value.

As used herein, term “analyte sensor,” “analyte biosensor,” or “sensor” refer to any device capable of receiving sensor information from a user, including for purpose of illustration but not limited to, body temperature sensors, blood pressure sensors, pulse or heart-rate sensors, glucose level sensors, analyte sensors, physical activity sensors, body movement sensors, or any other sensors for collecting physical or biological information. Analytes measured by the analyte sensors can include, by way of example and not limitation, glutamate, glucose, ketones, lactate, oxygen, hemoglobin A1C, albumin, alcohol, alkaline phosphatase, alanine transaminase, aspartate aminotransferase, bilirubin, blood urea nitrogen, calcium, carbon dioxide, chloride, creatinine, hematocrit, aspartate, asparagine, magnesium, oxygen, pH, phosphorus, potassium, sodium, total protein, uric acid, etc.

As used herein, the term “at least” prior to a number or series of numbers is understood to include the number associated with the term “at least,” and all subsequent numbers or integers that could logically be included, as clear from context. When at least is present before a series of numbers or a range, it is understood that “at least” can modify each of the numbers in the series or range. For example, “at least 3” means at least 3, at least 4, at least 5, etc. When at least is present before a component in a method step, then that component is included in the step, whereas additional components are optional.

As used herein, the term “polyvinylpyridine-based polymer” refers to a polymer (e.g., a copolymer) that includes polyvinylpyridine (e.g., poly(2-vinylpyridine) or poly(4-vinylpyridine)) or a substituted derivative thereof.

As used herein, the term “working electrode” is an electrode at which the analyte (or a second compound whose level depends on the level of the analyte) is electrooxidized or electroreduced with or without the agency of an electron transfer agent.

As used herein, the term “counter electrode” refers to an electrode paired with the working electrode, through which passes a current equal in magnitude and opposite in sign to the current passing through the working electrode. In the context of aspects of the present disclosure, the term “counter electrode” includes both a) counter electrodes and b) counter electrodes that also function as reference electrodes (i.e., counter/reference electrodes), unless otherwise indicated.

As used herein, the term “reference electrode” includes both a) reference electrodes and b) reference electrodes that also function as counter electrodes (i.e., counter/reference electrodes), unless otherwise indicated.

As used herein, the term “electrolysis” is the electrooxidation or electroreduction of a compound either directly at an electrode or via one or more electron transfer agents.

As used herein, components are “immobilized” or “attached” to a polymer and/or a sensor, for example, when the components are entrapped on, entrapped within, covalently bound, ionically bound, electrostatically bound, or coordinatively bound to constituents of a polymer, a sol-gel matric, membrane, and/or sensor, which reduces or precludes mobility.

As used herein, the term “non-leachable” compound, or a compound that is “non-leachably disposed” is meant to define a compound that is affixed on the sensor such that it does not substantially diffuse away from the sensing layer of the working electrode for the period in which the sensor is used (e.g., the period in which the sensor is implanted in a patient or measuring a sample).

As used herein, the term “electron transfer agent” is a compound that carries electrons between the analyte and the working electrode, either directly, or in cooperation with other electron transfer agents. One example of an electron transfer agent is a redox mediator.

As used herein, the term “redox mediator” is an electron-transfer agent for carrying electrons between an analyte, an analyte-reduced enzyme or analyte-oxidizedenzyme, and an electrode, either directly, or via one or more additional electron-transfer agents. A redox mediator that includes a polymeric backbone can also be referred to as a “redox polymer.”

As used herein, the term “precursor polymer” refers to a starting polymer before various modifier groups are attached to form a modified polymer.

As used herein, the term “substituted” when used to modify a functional group (e.g., substituted alkyl, substituted alkenyl, substituted alkoxy, substituted aryl) includes at least one substituent (e.g., 1, 2, 3, 4, or 5) that can be, for example, halo, alkoxy, mercapto, aryl, alkoxycarbonyl, alkylaminocarbonyl, dialkylaminocarbonyl, hydroxy, amino, alkylamino, dialkylamino, trialkylammonium, alkanoylamino, arylcarboxamido, hydrazino, alkylthio, alkenyl, and reactive groups.

As used herein, the term “reactive group” is a functional group of a molecule (e.g., a polymer, a crosslinking agent, an enzyme) that is capable of reacting with another compound to couple at least a portion (e.g., another reactive group) of that other compound to the molecule. Reactive groups include carboxy, activated ester, sulfonyl halide, sulfonate ester, isocyanate, isothiocyanate, epoxide, aziridine, halide, aldehyde, ketone, amine, acrylamide, thiol, acyl azide, acyl halide, hydrazine, hydroxylamine, alkyl halide, imidazole, pyridine, phenol, alkyl sulfonate, halotriazine, imido ester, maleimide, hydrazide, hydroxy, and photo-reactive azido aryl groups. Activated esters, as understood in the art, generally include esters of succinimidyl, benzotriazolyl, or aryl substituted by electron-withdrawing groups such as sulfo, nitro, cyano, or halo groups; or carboxylic acids activated by carbodiimides.

As used herein, the term “sensing layer” is a region or component of the sensor including constituents that facilitate the electrolysis of the analyte. The sensing layer can include constituents such as a redox mediator (e.g., an electron transfer agent or a redox polymer), a catalyst (e.g., an analyte-specific enzyme), which catalyzes a reaction of the analyte to produce a response at the working electrode, or both an electron transfer agent and a catalyst. In some aspects of the present disclosure, a sensor includes a sensing layer that is non-leachably disposed in proximity to or on the working electrode.

As used herein, the term “sensing element” is an application or region of an analyte-specific enzyme disposed with the sensing layer. As such, a sensing element is capable of interacting with the analyte. A sensing layer can have more than one sensing element making up the analyte detection area disposed on the working electrode. In some aspects, the sensing element includes an analyte-specific enzyme and an electron transfer agent (e.g., electron transfer agent). In some aspects, the sensing element includes an analyte specific enzyme, a redox mediator, and a crosslinker.

As used herein, the term “crosslinking agent” or “crosslinker” is a molecule that contains at least two (e.g., 2, 3, or 4) reactive groups (e.g., terminal functional groups) that can link at least two molecules together (intermolecular crosslinking) or at least two portions of the same molecule together (intramolecular crosslinking). A crosslinking agent having more than two reactive groups can be capable of both intermolecular and intramolecular crosslinkings at the same time.

As used herein, the term “bodily fluid” is any fluid or fluid derivative from a host/patient/subject in which an analyte of interest can be measured. Examples of a bodily fluid include, for example, dermal fluid, subcutaneous fluid, interstitial fluid, plasma, blood (e.g., from a vein or blood vessel), lymph, synovial fluid, cerebrospinal fluid, saliva, bronchoalveolar lavage, amniotic fluid, sweat, or tears. In certain aspects, the bodily fluid is dermal fluid or interstitial fluid.

As used herein, the term “patient” refers to a living animal, and thus encompasses a living mammal and a living human, for example. The term “user” can be used herein as a term that encompasses the term “patient.”

As used herein, the term “C₆₋₃₀ aryl” refers to an aromatic compound comprising a mono-, bi-, or tricyclic carbocyclic ring system having one, two, or three aromatic rings, for example, phenyl, naphthyl, anthracenyl, or biphenyl. The aromatic compound generally contains from, for example, 6 to 30 carbon atoms, from 6 to 18 carbon atoms, from 6 to 14 carbon atoms, or from 6 to 10 carbon atoms. It is understood that the term aryl includes carbocyclic moieties that are planar and comprise 4n+2 π electrons, according to Hückel's Rule, wherein n=1, 2, or 3.

As used herein, the term “halo” refers to a radical of a halogen, i.e., F, Cl, Br, or I.

As used herein, the term “C₁₋₆ alkyl” refers to a straight-chain or branched alkyl substituent containing from, for example, from about 1 to about 6 carbon atoms, e.g., from about 1 to about 4 carbon atoms or about 1 to about 3 carbons. Examples of alkyl group include, but are not limited to, methyl, ethyl, n-propyl, isopropyl, n-butyl, sec-butyl, isobutyl, tert-butyl, n-pentyl, isopentyl, n-hexyl, and the like. This definition also applies wherever “alkyl” occurs as part of a group, such as, e.g., C₁₋₆ haloalkyl (e.g., -trifluoromethyl (—CF₃)).

As used herein, the term “C₂₋₆ alkenyl” refers to a linear alkenyl substituent containing from, for example, 2 to about 6 carbon atoms (branched alkenyls are about 3 to about 6 carbons atoms). In accordance with an aspect, the alkenyl group is a C₂₋₄ alkenyl. Examples of alkenyl group include, but are not limited to, ethenyl, allyl, 2-propenyl, 1-butenyl, 2-butenyl, 1-pentenyl, 2-pentenyl, 3-pentenyl, 1-hexenyl, and the like.

As used herein, the term “C₂₋₆ alkynyl” refers to a linear alkynyl substituent containing from, for example, 2 to about 6 carbon atoms (branched alkynyls are about 3 to about 6 carbons atoms). In accordance with an aspect, the alkynyl group is a C₂₋₄ alkynyl. Examples of alkynyl group include, but are not limited to, ethynyl, propynyl, 1-butynyl, 2-butynyl, 1-pentynyl, 2-pentynyl, 3-pentynyl, 1-hexynyl, and the like.

As used herein, the term “hydroxy” refers to —OH.

As used herein, the term “nitro” refers to —NO₂.

As used herein, the term “cyano” refers to —CN.

As used herein, the term “amino” refers to —NH₂. The terms mono- and di-C₁₋₆ alkylamino refer to a nitrogen bonded to one or two C₁₋₆ alkyl groups, respectively, i.e., —NHR or —NRR′, in which R and R′ are the same or different C₁₋₆ alkyl groups.

As used herein, the term “C₁₋₆ alkoxy” refers to a C₁₋₆ alkyl group bonded to an oxygen, i.e., —OR, in which R is a C₁₋₆ alkyl group.

As used herein, the term “C₆₋₁₀ aryloxy” refers to an aryl group bonded to an oxygen, i.e., —O(Ar), in which Ar is a C₆₋₁₀ aryl group.

As used herein, the term “aralkoxy” refers to the group —OR(Ar), in which R is an C₁₋₆ alkyl group and Ar is a C₆₋₁₀ aryl group.

As used herein, the term “carboxy” refers to —C(O)OH.

As used herein, the term “C₁₋₆ alkylcarboxy” refers to a carboxy group wherein the hydrogen bound to the carboxy group has been replaced with a C₁₋₆ alkyl group, i.e., —C(O)OR, wherein R is an C₁₋₆ alkyl group.

As used herein, the term “amido” refers to the structure —C(O)NH or —NHC(O). The term “C₁₋₆ alkylamido” refers to —C(O)NR or —NRC(O), wherein R is C₁₋₆ alkyl.

As used herein, the term “C₁₋₆ haloalkylamido” refers to a C₁₋₆ alkylamido group in which the C₁₋₆ alkyl group is substituted with 1, 2, or 3 halo groups, as described herein.

As used herein, the term “heteroaryl” refers to an aromatic compound, as described herein, containing a 5 or 6 membered ring in which 1 or 2 carbons have been replaced with nitrogen, sulfur, and/or oxygen. Examples of heteroaryl include, but are not limited to, pyridinyl, furanyl, pyrrolyl, quinolinyl, thiophenyl, indolyl, oxazolyl, isoxazolyl, pyrazolyl, imidazolyl, thiazolyl, isothiazolyl, 1,3,4-thiadiazolyl, pyridazinyl, pyrimidinyl, pyrazinyl, and triazinyl.

As used herein, the term “heterocycloalkyl” refers to a monocyclic, bicyclic, or spiro ring system containing 3 to 7 carbon atom ring members and 1, 2, or 3 other atoms selected from nitrogen, sulfur, and/or oxygen. Examples of such heterocycloalkyl rings include, but are not limited to, aziridinyl, oxiranyl, thiazolinyl, imidazolidinyl, piperazinyl, homopiperazinyl, pyrrolinyl, pyrrolidinyl, tetrahydrofuranyl, tetrahydrothiofuranyl, pyranyl, tetrahydropyranyl, piperidinyl, and morpholinyl.

### Sensor

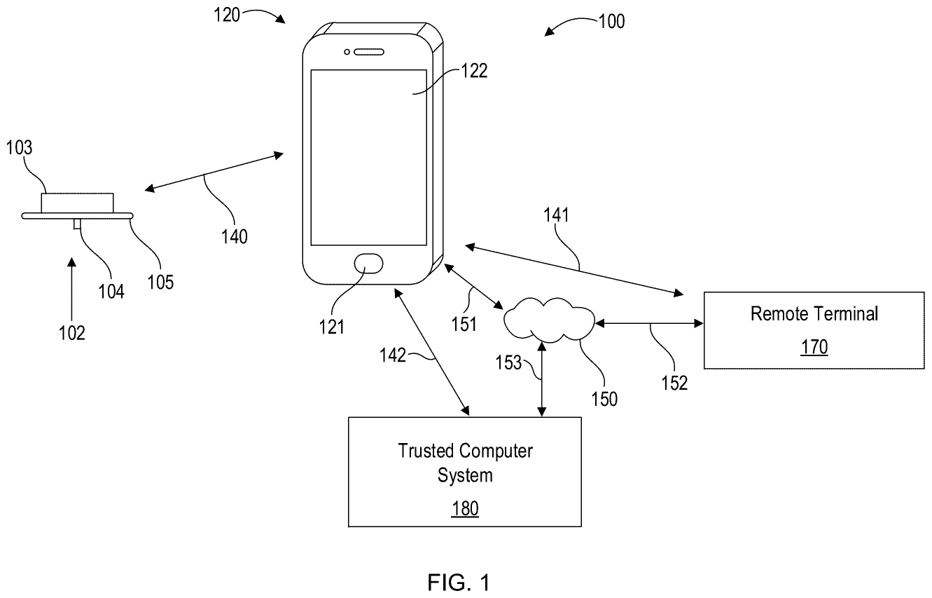

Before describing the analyte sensors of the present disclosure and their components in further detail, a brief overview of suitable in vivo analyte sensor configurations and sensor systems employing the analyte sensors will be provided so that the aspects of the present disclosure can be better understood. FIG. 1 shows a diagram of an illustrative sensing system that can incorporate an analyte sensor of the present disclosure. As shown, sensing system 100 includes sensor control device 102 and reader device 120 that are configured to communicate with one another over a local communication path or link 140, which can be wired or wireless, uni- or bi-directional, and encrypted or non-encrypted. Reader device 120 can constitute an output medium for viewing analyte concentrations and alerts or notifications determined by sensor 104 or a processor associated therewith, as well as allowing for one or more user inputs, according to certain aspects. Reader device 120 can be a multi-purpose smartphone or a dedicated electronic reader instrument. While only one reader device 120 is shown, multiple reader devices 120 can be present in certain instances. Reader device 120 can also be in communication with remote terminal 170 and/or trusted computer system 180 via communication path(s)/link(s) 141 and/or 142, respectively, which also can be wired or wireless, uni- or bi-directional, and encrypted or non-encrypted. Reader device 120 can also or alternately be in communication with network 150 (e.g., a mobile telephone network, the internet, or a cloud server) via communication path/link 151. Network 150 can be further communicatively coupled to remote terminal 170 via communication path/link 152 and/or trusted computer system 180 via communication path/link 153. Alternately, sensor 104 can communicate directly with remote terminal 170 and/or trusted computer system 180 without an intervening reader device 120 being present. For example, but not by the way of limitation, sensor 104 can communicate with remote terminal 170 and/or trusted computer system 180 through a direct communication link to network 150, according to certain aspects, as described in U.S. Patent Application Publication 2011/0213225 and incorporated herein by reference in its entirety. Any suitable electronic communication protocol can be used for each of the communication paths or links, such as near field communication (NFC), radio frequency identification (RFID), BLUETOOTH® or BLUETOOTH® Low Energy protocols, WiFi, or the like. Remote terminal 170 and/or trusted computer system 180 can be accessible, according to certain aspects, by individuals other than a primary user who have an interest in the user's analyte levels. Reader device 120 can include display 122 and optional input component 121. Display 122 can include a touch-screen interface, according to certain aspects.

Sensor control device 102 includes sensor housing 103, which can house circuitry and a power source for operating sensor 104. Optionally, the power source and/or active circuitry can be omitted. A processor (not shown) can be communicatively coupled to sensor 104, with the processor being physically located within sensor housing 103 or reader device 120. Sensor 104 protrudes from the underside of sensor housing 103 and extends through adhesive layer 105, which is adapted for adhering sensor housing 103 to a tissue surface, such as skin, according to certain aspects.

Sensor 104 is adapted to be at least partially inserted into a tissue of interest, such as within the dermal or subcutaneous layer of the skin. Sensor 104 can include an implantable or distal portion of sufficient length for insertion to a desired depth in a given tissue. In some aspects, the sensor can comprise a proximal portion configured to be positioned above a user's skin and a distal (or implantable) portion configured to be transcutaneously positioned beneath or through the user's skin and in contact with a bodily fluid to detect the analyte in vivo. In some aspects, the distal portion is configured to detect an analyte in the bodily fluid. In some aspects, the proximal portion can be electrically coupled with processing electronics. In some aspects, the processing electronics are disposed in the electronics housing of the sensor control device.

The implantable or distal portion can include at least one working electrode. In certain configurations, the implantable or distal portion can include a sensing layer for detecting an analyte (e.g., glucose). A counter electrode can be present in combination with the at least one working electrode. Particular electrode configurations upon the implantable or distal portion are described in more detail below.

The sensing layer can be configured for detecting a particular analyte (e.g., glucose). For example, but not by way of limitation, the disclosed analyte sensors include at least one sensing layer configured to detect an analyte (e.g., glucose).

In certain aspects of the present disclosure, an analytes (e.g., glucose) can be monitored in any biological fluid of interest such as dermal fluid, interstitial fluid, plasma, blood, lymph, synovial fluid, cerebrospinal fluid, saliva, bronchoalveolar lavage, amniotic fluid, or the like. In certain particular aspects, analyte sensors of the present disclosure can be adapted for assaying dermal fluid or interstitial fluid to determine a concentration of one or more analytes in vivo. In certain aspects, the biological fluid is interstitial fluid.

Referring still to FIG. 1, sensor 104 can automatically forward data to reader device 120. For example but not by the way of limitation, analyte concentration data (i.e., glucose concentration) can be communicated automatically and periodically, such as at a certain frequency as data is obtained or after a certain time period has passed, with the data being stored in a memory until transmittal (e.g., every minute, five minutes, or other predetermined time period). In certain other aspects, sensor 104 can communicate with reader device 120 in a non-automatic manner and not according to a set schedule. For example, but not by the way of limitation, data can be communicated from sensor 104 using RFID technology when the sensor electronics are brought into communication range of reader device 120. Until communicated to reader device 120, data can remain stored in a memory of sensor 104. Thus, a user does not have to maintain close proximity to reader device 120 at all times, and can instead upload data at a convenient time. In certain other aspects, a combination of automatic and non-automatic data transfer can be implemented. For example, and not by the way of limitation, data transfer can continue on an automatic basis until reader device 120 is no longer in communication range of sensor 104.

An introducer can be present transiently to promote introduction of sensor 104 into a tissue. In certain illustrative aspects, the introducer can include a needle or similar sharp. As would be readily recognized by a person skilled in the art, other types of introducers, such as sheaths or blades, can be present in alternative aspects. More specifically, the needle or other introducer can transiently reside in proximity to sensor 104 prior to tissue insertion and then be withdrawn afterward. While present, the needle or other introducer can facilitate insertion of sensor 104 into a tissue by opening an access pathway for sensor 104 to follow. For example, and not by the way of limitation, the needle can facilitate penetration of the epidermis as an access pathway to the dermis to allow implantation of sensor 104 to take place, according to one or more aspects. After opening the access pathway, the needle or other introducer can be withdrawn so that it does not represent a sharps hazard. In certain aspects, suitable needles can be solid or hollow, beveled or non-beveled, and/or circular or non-circular in cross-section. In more particular aspects, suitable needles can be comparable in cross-sectional diameter and/or tip design to an acupuncture needle, which can have a cross-sectional diameter of about 250 microns. However, suitable needles can have a larger or smaller cross-sectional diameter if needed for certain particular applications.

In certain aspects, a tip of the needle (while present) can be angled over the terminus of sensor 104, such that the needle penetrates a tissue first and opens an access pathway for sensor 104. In certain aspects, sensor 104 can reside within a lumen or groove of the needle, with the needle similarly opening an access pathway for sensor 104. In either case, the needle is subsequently withdrawn after facilitating sensor insertion.

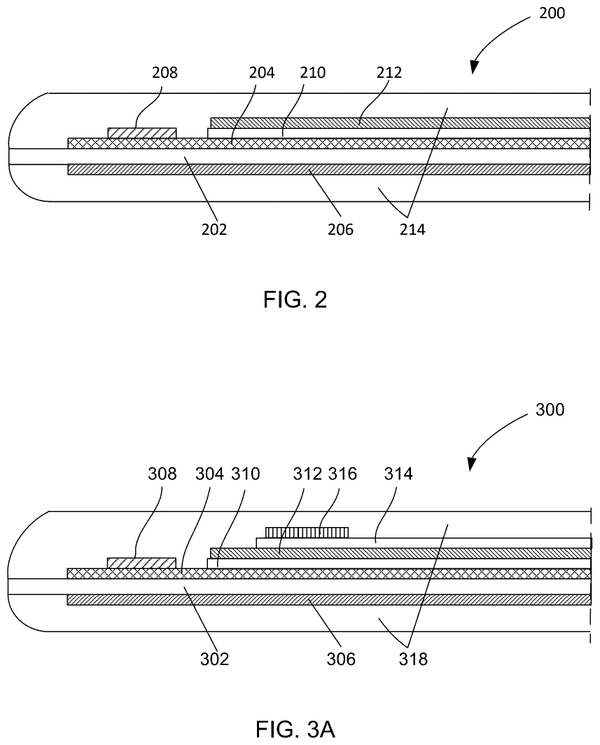

FIG. 2 illustrates a cross-sectional diagram of an implantable portion of an analyte sensor (e.g., a distal portion positionable below the surface of the skin) according to one or more aspects of the present disclosure. As shown in FIG. 2, the analyte sensor can include an implantable portion 200 including: (i) a substrate 202; (ii) a working electrode 204 on the substrate 202; (ii) a sensing layer 208 disposed upon a surface of the working electrode for detecting an analyte; (iii) a counter electrode 206 on the substrate 202; (iv) a dielectric (insulating) layer 210; (v) a reference electrode 212; and (vi) a polymer membrane 214 overcoating at least the sensing layer 208 and optionally a hydrogel coating (not shown) disposed on (e.g., overcoating) the polymer membrane 214.

In one or more aspects, as shown in FIG. 2, the substrate 202 can be disposed between the working electrode 204 and the counter electrode 206. In one or more aspects, different from aspects shown in FIG. 2, the working electrode 204 and the counter electrode 206 can be located upon the same side of the substrate 202 with a dielectric material (e.g., insulating layer) interposed therebetween.

The sensing layer 208 can be disposed as at least one layer upon the working electrode 204. The sensing layer 208 can include an active area such as a sensing spot configured to detect an analyte through sensing chemistry.

The sensing layer 208 can include a plurality of sensing spots on the working electrode 204. The plurality of sensing spots can be responsive to different analytes and are laterally spaced apart from one another on the surface of the working electrode 204. In some aspects, at least some of the plurality of sensing spots can be responsive to a same analyte, and the rest can be responsive to different analytes. The membrane 214 can cover a portion of the plurality of sensing spots or all of the plurality of sensing spots. The plurality of sensing spots can be configured to detect their corresponding analytes at working electrode potentials that differ from one another.

In one or more aspects, the dielectric (insulating) layer 210 can be disposed between the working electrode 204 and the reference electrode 212. In one or more aspects, different from the configuration shown in FIG. 2, the reference electrode 212 can be the side of the counter electrode 206 with the dielectric (insulating) layer 210 interposed therebetween. The dielectric (insulating) layer 210 separates the working electrode 204 and the reference electrode 212, or separates the counter electrode 206 and the reference electrode 212, from each other to provide electrical isolation.

In one or more aspects, the reference electrode 212 can be a silver/silver chloride electrode.

The polymer membrane 214 overcoats at least the sensing layer 208. In one or more aspects, the polymer membrane 204 can overcoat some or all of the working electrode 204, the counter electrode 206, and the reference electrode 212, or the entirety of the implantable portion 200 of the analyte sensor. One or both faces of the implantable portion 200 of the analyte sensor can be overcoated with polymer membrane 214. The polymer membrane 214 can include one or more polymeric membrane materials, such as a polymer matrix, having capabilities of limiting analyte flux to the sensing layer 208 (e.g., the polymer membrane 214 is a diffusion-limiting membrane having some permeability for an analyte of interest). In one or more aspects, the polymer membrane 214 can be crosslinked with a crosslinker (e.g., a branched crosslinker) in certain particular sensor configurations. The composition and thickness of the polymer membrane 214 can vary to promote a desired or suitable analyte flux to the sensing layer 208, thereby providing a desired or suitable signal intensity and stability. The analyte sensor can be operable for assaying an analyte by any of coulometric, amperometric, voltammetric, or potentiometric electrochemical detection techniques. In some aspects, the polymer membrane 214 comprises at least one antimicrobial agent (e.g., an antibiotic). In some aspects, the polymer membrane 214 has a hydrogel coating disposed thereon. In some aspects, the polymer membrane 214 comprises both at least one antimicrobial agent (e.g., an antibiotic) and a hydrogel coating disposed thereon.

Several parts of the sensor are further described below.

In an aspect, the present disclosure is directed to an analyte sensor comprising
a working electrode,
a sensing layer disposed on at least a portion of the working electrode that comprises an analyte-responsive enzyme, and
a polymer membrane overcoating at least the sensing layer,
wherein the polymer membrane comprises an antimicrobial agent disposed therein, a hydrogel coating disposed thereon, or both an antimicrobial agent disposed therein and hydrogel coating disposed thereon.

The present disclosure further relates to an analyte sensor for detecting an analyte in vivo, the sensor comprising:
a proximal portion configured to be positioned above a user's skin; and
a distal portion configured to be transcutaneously positioned beneath the skin and in contact with bodily fluid to detect the analyte in vivo;
the distal portion comprising:
   a working electrode,
   a sensing layer disposed on at least a portion of the working electrode that comprises an analyte-responsive enzyme, and
   a polymer membrane overcoating at least the sensing layer,
      wherein the polymer membrane comprises an antimicrobial agent disposed therein, a hydrogel coating disposed thereon, or both an antimicrobial agent disposed therein and hydrogel coating disposed thereon.

In some aspects, the proximal portion is electrically coupled with a processor configured to (a) correlate a signal indicative of analyte concentration obtained by the sensor to analyte concentration in the bodily fluid; and to (b) communicate the analyte concentration to a reader device to be displayed.

It was discovered that the presence of an antimicrobial agent, such as one or more antibiotics, a hydrogel coating, or both reduces early/late signal attenuation of the analyte sensor. In an aspect, the analyte sensor has reduced early and/or late signal attenuation relative to the same sensor without an antimicrobial agent (e.g., one or more antibiotics) disposed in the polymer membrane, a hydrogel coating disposed on the polymer membrane, or both. The reduction in early and/or late signal attenuation can be to any suitable degree, such as about 10% reduction or more, about 15% reduction or more, about 20% reduction or more, about 25% reduction or more, about 30% reduction or more, about 35% reduction or more, about 40% reduction or more, about 45% reduction or more, about 50% reduction or more, about 55% reduction or more, about 60% reduction or more, about 65% reduction or more, about 70% reduction or more, about 80% reduction or more, about 85% reduction or more, or about 90% reduction or more.

The polymer membrane is disposed on at least the sensing layer. In some aspects, the polymer membrane is prepared by forming a first polymer layer on the sensing layer (e.g., via dip coating) and then forming one or more subsequent polymer layers, in which one or more of the subsequent layers has antimicrobial properties (e.g., contains an antimicrobial agent). In some aspects, the polymer membrane is prepared by forming a layer with antimicrobial properties directly adjacent the sensing layer.

In the analyte sensor, at least one working electrode is present. In some aspects, one working electrode is present. In some aspects, two or more working electrodes are present. A working electrode can be any suitable conductive material. Examples of suitable conductive materials include, e.g., aluminum, carbon (including graphite), cobalt, copper, gallium, gold, indium, iridium, iron, lead, magnesium, mercury (as an amalgam), nickel, niobium, osmium, palladium, platinum, rhenium, rhodium, selenium, silicon (e.g., doped polycrystalline silicon), silver, tantalum, tin, titanium, tungsten, uranium, vanadium, zinc, zirconium, mixtures thereof, and alloys, oxides, or metallic compounds of these elements. In some aspects, a working electrode can comprise carbon.

In addition to at least one working electrode (e.g., a first working electrode, a second working electrode), a sensing layer disposed on at least a portion of the working electrode, and a polymer membrane overcoating at least the sensing layer, the analyte sensor further can comprise a reference electrode, a counter electrode, or both a reference electrode and a counter electrode in some aspects. In an aspect, the counter electrode can be carbon (e.g., screen-printed carbon), and the reference electrode can be Ag/AgCl. In a two electrode example, a working electrode and a second electrode that functions as both a counter electrode and reference electrode (i.e., a counter/reference electrode) can be used.

In some aspects, the analyte sensor can comprise at least one dielectric (e.g., insulating) layer. In some aspects, the dielectric (e.g., insulating) layer can be comprised of a suitable dielectric material that can form a solid. In an example, the dielectric (e.g., insulating) layer can be formed from porcelain (ceramic), mica, glass, barium strontium titanate, a plastic (e.g., polystyrene, polytetrafluoroethylene, polyethylene terephthalate, polyethylene, polypropylene, polymethylmethacrylate, polysulfone, polydimethylsiloxane, polyvinyl chloride, or a combination thereof), or a metal oxide (e.g., silica, alumina, titania, zirconia, tantalum oxide, etc.).

In some aspects, the analyte sensor is exposed to a bodily fluid in vivo. In general, the method uses an analyte sensor, as disclosed herein, for measuring a concentration of an analyte (e.g., glucose) and can be used in an in vivo monitoring system, which while positioned in vivo in a user (e.g., a patient, such as a human) makes contact with the bodily fluid of the user and senses an analyte contained therein. An in vivo analyte monitoring system can include one or more reader devices that receives sensed analyte data from a sensor control device. In an aspect, the sensor control device is configured to determine data indicative of analyte concentration and to transmit the data indicative of analyte concentration to a reader device according to an electronic communication protocol via a transmitter coupled to the sensor control device. The reader device can process and/or display the sensed analyte data or sensor data in any number of forms to the user. In some aspects, the reader device can be a mobile communication device, such as a dedicated reader device (configured for communication with a sensor control device) optionally in conjunction with a computer system, a mobile telephone (e.g., a WiFi or internet-enabled smart phone), a tablet, a personal digital assistant (PDA), or a mobile smart wearable electronics assembly (e.g., a smart glass, smart glasses, watch, bracelet, or necklace). Configuring a reader device to an in vivo monitoring system is described at, for example, U.S. Pat. No. 11,371,957, the disclosure of which is incorporated herein by reference in its entirety.

The reader device typically includes an input component, a display, and processing circuitry, which can include one or more processors, microprocessors, controllers, and/or microcontrollers, each of which can be a discrete chip or distributed amongst (and a portion of) a number of different chips. The processing circuitry can include a communications processor having on-board memory and an applications processor having on-board memory. The reader device can further include radio frequency (RF) communication circuitry coupled with an RF antenna, a memory, multi-functional circuitry with one or more associated antennas, a power supply, power management circuitry, and/or a clock. In an aspect, the analyte monitoring system can include a power source for operating the sensor control device. It will be recognized that other hardware and functionality can be included in the reader device.

The analyte sensor and/or any other relevant devices or components according to aspects of the present disclosure described herein can be implemented utilizing any suitable hardware, firmware (e.g., an application-specific integrated circuit), software, or a combination of software, firmware, and hardware. For example, the various components of the sensor can be formed on one integrated circuit (IC) chip or on separate IC chips. Further, the various components of the sensor can be implemented on a flexible printed circuit film, a tape carrier package (TCP), a printed circuit board (PCB), or formed on one substrate. Further, the various components of the sensor can be a process or thread, running on one or more processors, in one or more computing devices, executing computer program instructions and interacting with other system components for performing the various functionalities described herein. The computer program instructions are stored in a memory which can be implemented in a computing device utilizing a standard memory device, such as, for example, a random access memory (RAM). The computer program instructions can also be stored in other non-transitory computer readable media such as, for example, a CD-ROM, flash drive, and/or the like. Also, a person of skill in the art will recognize that the functionality of one or more suitable computing devices can be combined or integrated into a single computing device, or the functionality of a particular computing device can be distributed across one or more other computing devices without departing from the scope of the example aspects of the present disclosure.

### Sensing Chemistry

The analyte sensors of the present disclosure can include one or more enzymes for detecting one or more analytes. Suitable enzymes for use in a sensor of the present disclosure can include, but are not limited to, enzymes for use in detecting glutamate, glucose, ketones, lactate, oxygen, hemoglobin A1C, albumin, alcohol, alkaline phosphatase, alanine transaminase, aspartate aminotransferase, bilirubin, blood urea nitrogen, calcium, carbon dioxide, chloride, creatinine, hematocrit, aspartate, asparagine, magnesium, oxygen, pH, phosphorus, potassium, sodium, total protein, and uric acid. In one or more aspects, analyte-responsive enzymes for use in detecting glucose, lactate, ketones, glutamate, pyruvate, creatinine, sarcosine, and/or alcohol (e.g., ethanol) can be included in a sensing layer of an analyte sensor disclosed herein. In some aspects, the one or more analyte-responsive enzymes can include multiple enzymes, e.g., an enzyme system, which are collectively responsive to the analyte. In some aspects, the enzyme is an oxidase enzyme or a dehydrogenase enzyme. Suitable examples of the analyte-responsive enzyme include glucose oxidase, glucose dehydrogenase, glutamate oxidase, lactate oxidase, lactate dehydrogenase, pyruvate oxidase, alcohol oxidase, xanthine oxidase, β-hydroxybutyrate dehydrogenase, 11β-hydroxysteroid dehydrogenase type 2 (11β-HSD-2), creatine amidohydrolase, sarcosine oxidase, nicotinamide adenine dinucleotide (NADH)-dependent oxidase, NADPH dehydrogenase, a flavin adenine dinucleotide (FAD)-dependent oxidase, a flavin mononucleotide (FMN)-dependent oxidase, diaphorase, catalase, and any combination thereof.

In one or more aspects, the sensing layer of an analyte sensor of the present disclosure can include one or more analyte-responsive enzymes that can be used to detect glucose. For example, but not by way of limitation, an analyte sensor of the present disclosure can include a sensing layer including a plurality of sensing spots, at least one of the sensing spots can include one or more enzymes for detecting glucose. In some aspects, the analyte sensor can include at least one sensing spot including a glucose oxidase and/or a glucose dehydrogenase for detecting glucose. In some aspects, the analyte sensor can include at least one sensing spot including a glucose oxidase.

In one or more aspects, one or more sensing spots of an analyte sensor of the present disclosure can include one or more enzymes that can be used to detect ketones. For example, but not by way of limitation, an analyte sensor of the present disclosure can include at least one sensing spot that includes one or more analyte-responsive enzymes, e.g., an enzyme system, for detecting ketones. In some aspects, the analyte sensor can include at least one sensing spot including β-hydroxybutyrate dehydrogenase. In some aspects, the analyte sensor can include at least one sensing spot including 3-hydroxybutyrate dehydrogenase and diaphorase for detecting ketones.

In one or more aspects, one or more sensing spots of an analyte sensor of the present disclosure can include one or more enzymes that can be used to detect lactate. For example, but not by way of limitation, an analyte sensor of the present disclosure can include a sensing spot includes one or more analyte-responsive enzymes, e.g., an enzyme system, for detecting lactate. In some aspects, the analyte sensor can include at least one sensing spot including a lactate dehydrogenase. In some aspects, the analyte sensor can include at least one sensing spot including a lactate oxidase.

In some aspects, the analyte-responsive enzyme is glutamate oxidase to detect glutamate. In some aspects, the analyte-responsive enzyme is pyruvate oxidase to detect pyruvate. In some aspects, the enzymes are alcohol oxidase and xanthine oxidase to detect ethanol or other alcohols. In some aspects, the analyte-responsive enzyme is creatine amidohydrolase and/or sarcosine oxidase to detect creatine and/or sarcosine.

If necessary, one or more cofactors can be included with the enzyme, which serves as a catalyst for the electron transfer. Suitable cofactors include, e.g., pyrroloquinoline quinone (PQQ), thiamine pyrophosphate (TPP), flavin adenine dinucleotide (FAD), nicotinamide adenine dinucleotide (NAD), and any combination thereof.

In one or more aspects, each sensing spot can be configured to detect the same analyte or a different analyte. For example, but not by way of limitation, an analyte sensor of the present disclosure can include a first sensing spot that includes a first enzyme (or a first enzyme system) for detecting a first analyte and a second sensing spot that includes a second enzyme (or a second enzyme system) for detecting a second analyte, and so on. In some aspects, the first sensing spot and the second sensing spot can be used to detect the same analyte, where the first sensing spot and the second sensing spot can include different enzymes (or enzyme system) or the same enzyme (or enzyme system) for detecting the analyte.

In one or more aspects, the sensing spot can further include a stabilizing agent, e.g., for stabilizing the one or more enzymes. For example, but not by way of limitation, the stabilizing agent can be an albumin, e.g., a serum albumin. Non-limiting examples of serum albumins can include bovine serum albumin and human serum albumin. In some aspects, the stabilizing agent can be a human serum albumin. In some aspects, the stabilizing agent can be a bovine serum albumin.

In certain aspects, the sensing layer can include a ratio of albumin stabilizer to enzyme from about 40:1 to about 1:40, e.g., from about 35:1 to about 1:35, from about 30:1 to about 1:30, from about 25:1 to about 1:25, from about 20:1 to about 1:20, from about 15:1 to about 1:15, from about 10:1 to about 1:10, from about 9:1 to about 1:9, from about 8:1 to about 1:8, from about 7:1 to about 1:7, from about 6:1 to about 1:6, from about 5:1 to about 1:5, from about 4:1 to about 1:4, from about 3:1 to about 1:3, from about 2:1 to about 1:2 or about 1:1. In certain aspects, the sensing layer can include a ratio of albumin stabilizer to enzyme from about 1:1 to about 1:10, e.g., from about 1:1 to about 1:9, from about 1:1 to about 1:8, from about 1:1 to about 1:7, from about 1:1 to about 1:6, from about 1:1 to about 1:5, from about 1:2 to about 1:9, from about 1:3 to about 1:8, from about 1:3 to about 1:7 or from about 1:4 to about 1:6.

In any of the aspects, the sensing layer can comprise a pH buffer. The buffer can be any suitable composition that is water soluble and controls (i.e., maintains) the pH of the sensing composition within a pH of about 5 to about 8 (e.g., maintains a pH of about 5, about 5.5, about 6, about 6.5, about 7, about 7.5, or about 8). In some aspects, the pH can be controlled to be within a range of about 6 to about 8. For example, the buffer can comprise a phosphate (e.g., monobasic and dibasic sodium phosphate), 4-(2-hydroxyethyl)piperazine-1-ethanesulfonic acid (HEPES), 3-(N-morpholino) propanesulfonic acid (MOPS), 2-amino-2-(hydroxymethyl)-1,3-propanediol (TRIS), a carbonate (e.g., carbonic acid and a carbonate salt, such as sodium carbonate; sodium carbonate and sodium bicarbonate), or a citrate (e.g., citric acid and a citrate salt, such as trisodium citrate). The buffer can optionally comprise one or more (e.g., 1, 2, 3, or 4) additional salts (e.g., Group I or Group II halide salts, e.g., sodium chloride, potassium chloride, magnesium chloride). In an aspect, the buffer can be phosphate-buffered saline (PBS), which comprises disodium hydrogen phosphate, sodium chloride, and optionally potassium chloride and potassium dihydrogen phosphate. In another aspect, the buffer can be HEPES or a phosphate buffer that can comprise phosphate, sodium chloride, and magnesium chloride.

The buffer typically is an aqueous buffer but other non-aqueous solvents can be present, such as an alcohol (e.g., ethanol). In some aspects, the buffer comprises water as the only solvent. In other aspect, the buffer can comprise water and at least one (e.g., 1, 2, or 3) non-aqueous solvents in any suitable ratio, such as a non-aqueous solvent to water volume ratio ranging from 99.9:0.1 to 0.1:99.9. In some aspects, the non-aqueous solvent to water volume ratio is about 1:99, about 5:95, about 10:90, about 15:85, about 20:80, about 25:75, about 30:70, about 35:65, about 40:60, about 45:55, about 50:50, about 55:45, about 60:40, about 65:35, about 70:30, about 75:25, about 80:20, about 85:15, about 90:10, about 95:5, or about 99:1, etc.). In a specific example, ethanol (EtOH) and water are used in a volume ratio ranging from 50:50 to 90:10 EtOH:H₂O (e.g., 70:30, about 75:25, about 80:20, about 85:15, or about 90:10, etc.).

In one or more aspects, an analyte sensor disclosed herein can include an electron transfer agent. For example, but not by way of limitation, one or more sensing spots of an analyte sensor can include an electron transfer agent. In one or more aspects, an analyte sensor can include one sensing spot that includes an electron transfer agent and a second sensing spot that does not include an electron transfer agent. In some aspects, an analyte sensor can include a plurality of sensing spots, where the plurality of sensing spots can include an electron transfer agent. In one or more aspects, the presence of an electron transfer agent in a sensing spot can depend on the enzyme or enzyme system used to detect the analyte and/or the composition of the working electrode.

Suitable electron transfer agents for use in the presently disclosed analyte sensors can facilitate conveyance of electrons to the adjacent working electrode after an analyte undergoes an enzymatic oxidation-reduction reaction within the corresponding sensing spot, thereby generating a current that is indicative of the presence of that particular analyte. The amount of current generated is proportional to the quantity of analyte that is present.

In one or more aspects, suitable electron transfer agents can include electroreducible and electrooxidizable ions, complexes, or molecules (e.g., quinones) having oxidation-reduction potentials that are a few hundred millivolts above or below the oxidation-reduction potential of a standard calomel electrode. The electron transfer agent typically comprises a transition metal complex. Suitable transition metal complexes can comprise osmium, ruthenium, iron, cobalt, vanadium, or a combination thereof. In some aspects, the transition metal can be ruthenium or osmium, particularly osmium. In some aspects, the redox mediators can include osmium complexes and other transition metal complexes, such as those described in U.S. Pat. Nos. 6,134,461, 6,605,200, 6,736,957, 7,501,053, and 7,754,093, which are incorporated herein by reference in their entireties. Additional examples of suitable redox mediators can include those described in U.S. Pat. Nos. 8,444,834, 8,268,143, and 6,605,201, the disclosures of each of which are also incorporated herein by reference in their entireties. Other examples of suitable redox mediators can include metal compounds or complexes of ruthenium, osmium, iron (e.g., polyvinylferrocene or hexacyanoferrate) or cobalt, including metallocene compounds thereof, for example.

The transition metal complex can further comprise at least one ligand, which can be monodentate or multidentate (e.g., bidentate, tridentate, tetradentate). Typically the complex will include enough ligands to provide a full coordination sphere. In some aspects, at least one ligand (e.g., 1, 2, 3, 4, 5, or 6) can comprise a nitrogen-containing heterocycle.

Monodentate ligands include, for example, —F, —Cl, —Br, —I, —CN, —SCN, —OH, NH₃, alkylamine, dialkylamine, trialkylamine, alkoxy, a heterocyclic compound, compounds containing such groups, a solvent molecule (e.g., H₂O, EtOH), or a reactive group. For example, an alkyl (e.g., C₁₋₁₂, C₁₋₆, C₁₋₄, C₁₋₃) or aryl (e.g., phenyl, benzyl, naphthyl) portions of a ligand can be optionally substituted by F, Cl, Br, I, alkylamino, dialkylamino, trialkylammonium (except aryl portions), alkoxy, alkylthio, aryl. Examples of suitable heterocyclic monodentate ligands include imidazole, pyrazole, oxazole, thiazole, pyridine, and pyrazine, each of which can be unsubstituted or substituted, as described herein (e.g., with at least one reactive group, such as 1, 2, 3, or 4 reactive groups).

Examples of suitable bidentate ligands include, for example, 1,10-phenanthroline, an amino acid, oxalic acid, acetylacetone, a diaminoalkane, an ortho-diaminoarene, 2,2′-biimidazole, 2,2′-bioxazole, 2,2′-bithiazole, 2-(2-pyridyl)imidazole, and 2,2′-bipyridine, each of which can be unsubstituted or substituted, as described herein (e.g., substituted with at least one reactive group, such as 1, 2, 3, or 4 reactive groups). Particularly suitable bidentate ligands for the electron transfer complex include substituted and unsubstituted 2,2′-biimidazole, 2-(2-pyridyl)imidazole, and 2,2′-bipyridine. Examples of suitable terdentate ligands include, for example, diethylenetriamine, 2,2′,2″-terpyridine, 2,6-bis(N-pyrazolyl)pyridine, each of which can substituted or unsubstituted (e.g., substituted with one more alkyl groups, such as methyl, or one or more reactive groups).

A suitable 2,2′-biimidazole ligand can be a ligand according to formula (I):

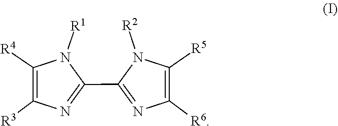

In formula (I), R¹ and R² are the same or different and each is a substituted or unsubstituted alkyl, alkenyl, or aryl. Generally, R¹ and R² are the same or different and each is an unsubstituted C₁₋₁₂ alkyl (e.g., C₁₋₄ alkyl). In some aspects, both R¹ and R² are methyl.

In formula (I), R³, R⁴, R⁵, and R⁶ are the same or different and each is H, F, Cl, Br, I, NO₂, CN, CO₂H, SO₃H, SH, alkoxycarbonyl, alkylaminocarbonyl, dialkylaminocarbonyl, hydroxy, alkoxy, amino, alkylamino, dialkylamino, alkanoylamino, arylcarboxamido, hydrazino, alkylhydrazino, hydroxylamino, alkoxyamino, alkylthio, alkyl, alkenyl, or aryl. Alternatively, R³ and R⁴, in combination, or R⁵ and R⁶, in combination, independently form a saturated or unsaturated 5- or 6-membered ring (e.g., benzo). Typically, the alkyl and alkoxy portions are C₁₋₁₂. The alkyl or aryl portions of any of the substituents can be optionally substituted by one or more substituents (e.g., 1, 2, 3, 4, 5, or 6), such as F, Cl, Br, I, amino, alkylamino, dialkylamino, trialkylammonium (except on aryl portions), alkoxy, alkylthio, aryl, or a reactive group (e.g., CO₂H). Generally, R³, R⁴, R⁵, and R⁶ are the same or different and each is H or an unsubstituted C₁₋₁₂ alkyl (e.g., C₁₋₄ alkyl). In some aspects, R³, R⁴, R⁵, and R⁶ are all H.

A suitable 2-(2-pyridyl)imidazole ligand can be a ligand according to formula (II):

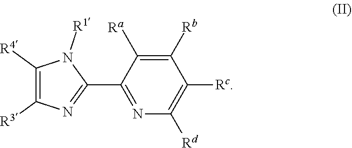

In formula (II), R¹ is a substituted or unsubstituted alkyl, alkenyl, or aryl. Generally, R¹ is an unsubstituted C₁₋₁₂ alkyl (e.g., C₁₋₄ alkyl) or a C₁₋₁₂ alkyl that is optionally substituted with a reactive group. In some aspects, R¹ is methyl.

In formula (II), R^{3′}, R^{4′}, R^{a}, R^{b}, R^{c}, and R^{d} are the same or different and each is H, F, Cl, Br, I, NO₂, CN, CO₂H, SO₃H, SH, alkoxycarbonyl, alkylaminocarbonyl, dialkylaminocarbonyl, hydroxy, alkoxy, amino, alkylamino, dialkylamino, alkanoylamino, arylcarboxamido, hydrazino, alkylhydrazino, hydroxylamino, alkoxyamino, alkylthio, alkyl, alkenyl, or aryl. Alternatively, R^{3′} and R^{4′}, in combination, or two adjacent substituents of R^{a}, R^{b}, R^{c}, and R^{d} (e.g., R^{a} and R^{b}, R^{b} and R^{c}, or R^{c} and R^{d}) in combination, independently form a saturated or unsaturated 5- or 6-membered ring (e.g., benzo). Typically, the alkyl and alkoxy portions are C₁₋₁₂. The alkyl or aryl portions of any of the substituents can be optionally substituted by one or more substituents (e.g., 1, 2, 3, 4, 5, or 6), such as F, Cl, Br, I, amino, alkylamino, dialkylamino, trialkylammonium (except on aryl portions), alkoxy, alkylthio, aryl, or a reactive group (e.g., CO₂H). Generally, R^{3′}, R^{4′}, R^{a}, R^{b}, R^{c}, and R^{d} are the same or different and each is H or an unsubstituted C₁₋₁₂ alkyl (e.g., C₁₋₄ alkyl). In some aspects, R^{3′}, R^{4′}, R^{a}, R^{b}, R^{c}, and R^{d} are all H.

A suitable 2,2′-bipyridine ligand can be a ligand according to formula (III):

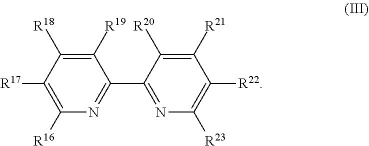

In formula (III), R¹⁶, R¹⁷, R¹⁸, R¹⁹, R²⁰, R²¹, R²², and R²³ are the same or different and each is H, F, Cl, Br, I, NO₂, CN, CO₂H, SO₃H, SH, alkoxycarbonyl, alkylaminocarbonyl, dialkylaminocarbonyl, hydroxy, alkoxy, amino, alkylamino, dialkylamino, alkanoylamino, arylcarboxamido, hydrazino, alkylhydrazino, hydroxylamino, alkoxyamino, alkylthio, alkyl, alkenyl, or aryl. Typically, the alkyl and alkoxy portions are C₁₋₁₂. The alkyl or aryl portions of any of the substituents can be optionally substituted by one or more substituents (e.g., 1, 2, 3, 4, 5, or 6), such as F, Cl, Br, I, amino, alkylamino, dialkylamino, trialkylammonium (except on aryl portions), alkoxy, alkylthio, aryl, or a reactive group (e.g., CO₂H).

Specific examples of suitable combinations include R¹⁶ and R²³ are both H or both methyl and/or R¹⁷ and R²³ are both H or both methyl and/or R¹⁸ and R²¹ are both H or both methyl and/or R¹⁹ and R²⁰ are both H or both methyl. An alternative combination is where one or more adjacent pairs of substituents (e.g., R¹⁶ and R¹⁷, R¹⁷ and R¹⁸, R¹⁸ and R¹⁹, R²³ and R²², R²² and R²¹, or R²¹ and R²⁰), in combination, form a saturated or unsaturated 5- or 6-membered ring (e.g., benzo).

In an aspect, the one or more ligand is 4,4′-dimethyl-2,2′-bipyridine, mono-, di-, or polyalkoxy-2,2′-bipyridines (e.g., 4,4′-dimethoxy-2,2′-bipyridine), 4,7-dimethyl-1,10-phenanthroline, mono, di-, or polyalkoxy-1,10-phenanthrolines (e.g., 4,7-dimethoxy-1,10-phenanthroline), or a combination of any of these.

In some aspects, the transition metal complex will include a counterion (X) to balance the charge of the transition metal. Typically, there will be 1 to 5 (i.e., 1, 2, 3, 4, or 5) counterions. Multiple counterions in the complex are not necessarily all the same. Examples of suitable counterions include anions, such as halide (e.g., fluoride, chloride, bromide, or iodide), sulfate, phosphate, hexafluorophosphate, and tetrafluoroborate, and cations (e.g., a monovalent cation), such as lithium, sodium, potassium, tetralkylammonium, and ammonium. In some aspects, the counterion is a halide, such as chloride.

In an aspect, the transition metal complex can be an osmium transition metal complex that can comprise one or more ligands, wherein at least one (e.g., 1, 2, 3, 4, 5, or 6) ligand that can comprise a nitrogen-containing heterocycle (e.g., imidazole, pyrazole, oxazole, thiazole, pyridine, and pyrazine). In some aspects, the osmium transition metal complex can comprise one or more ligands selected from 4,4′-dimethyl-2,2′-bipyridine, mono-, di-, or polyalkoxy-2,2′-bipyridines (e.g., 4,4′-dimethoxy-2,2′-bipyridine), 4,7-dimethyl-1,10-phenanthroline, mono, di-, or polyalkoxy-1,10-phenanthrolines (e.g., 4,7-dimethoxy-1,10-phenanthroline).

In an aspect, the redox mediator can comprise an osmium complex bonded to a polymer or copolymer formed from poly(1-vinyl imidazole) or poly(4-vinylpyridine). The poly(4-vinylpyridine)-based polymer is a prepolymer that has been modified, as shown in the following structure, to attach an osmium complex (e.g., a poly(biimidizyl) osmium complex).

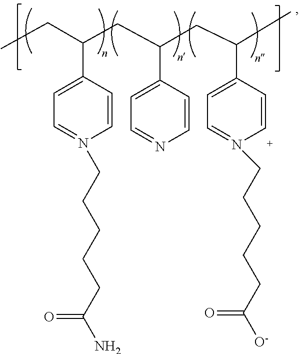

wherein n can be 2, n′ can be 17, and n″ can be 1. Other reactive groups and/or spacer groups can be used.

In an aspect, the electron redox mediator can comprise an osmium-containing poly(4-vinylpyridine)-based polymer, as shown below.

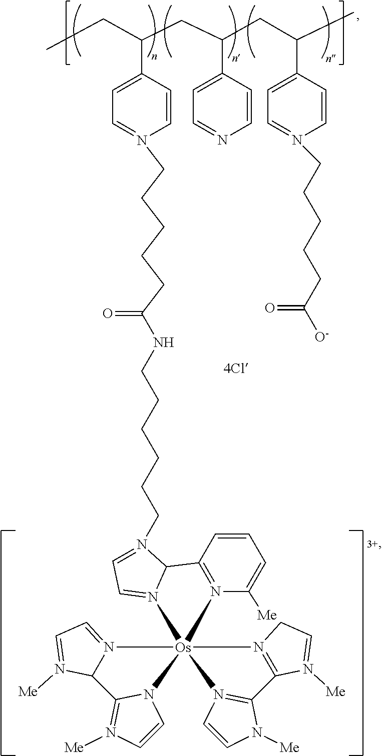

wherein n is 2, n′ is 17, and n″ is 1.

The electron transfer agent typically is attached (e.g., non-leachable and/or covalently bonded) to the polymer. For example, covalent bonding of the electron transfer agent to the polymer can take place by polymerizing a monomer unit bearing a covalently bound electron transfer agent, or the electron transfer agent can be reacted with the polymer separately after the polymer has already been synthesized.

According to some aspects, a bifunctional spacer can be used to attached (e.g., covalently bond) the electron transfer agent to the polymer, with a first reactive group being reactive with the polymer (e.g., a functional group capable of quaternizing a pyridine nitrogen atom or an imidazole nitrogen atom) and a second reactive group being reactive with the electron transfer agent (e.g., a functional group that is reactive with a ligand coordinating a metal ion). Typically, covalent bonds are formed between the two reactive groups to generate a linkage. Suitable reactive groups include, for example, activated ester (e.g., succinimidyl, benzotriazolyl, or an aryl substituted with one more electron withdrawing groups, such as sulfo, nitro, cyano, or halo), acrylamido, acyl azido, acyl halide, carboxy (—COO— or —CO₂H), aldehyde, ketone, alkyl halide, alkyl sulfonato, anhydride, aziridino, epoxy, halotriazinyl, imido ester, isocyanato, isothiocyanato, maleimido, sulfonyl halide, amino, thiol (—SH), hydroxy, pyridinyl, imidazolyl, and hydroxyamino. The reaction between two reactive groups can form a covalent linkage between the transition metal complex and the polymer that is a carboxamido, thioether, hydrazonyl, oximyl, alkyamino, ester, carboxylic ester, imidazolium, pyridinium, ether, thioether, aminotriazinyl, triazinyl ether, amidinyl, urea, urethanyl, thiourea, thioether, sulfonamide, or any combination. In addition to the reactive groups, the bifunctional spacer typically can further comprise an alkylenyl (i.e., —(CH₂)ₙ—) and/or ethylenyloxy (i.e., —(CH₂CH₂O)ₘ—, in which n and m are each independently an integer from 1 to 12 (e.g., 1 to 11, 1 to 10, 1 to 9, 1 to 8, 1 to 7, 1 to 6, 1 to 5, 1 to 4, 1 to 3, or 1 to 2).

In some aspects, the sensing layer, and in particular, the redox mediator can further comprise a cross linking agent to form a crosslinked polymer. In general, the cross linking agent is any suitable multifunctional (e.g., bifunctional) short chain molecule that enables the electron transfer agent to attach (e.g., covalently bond) to the polymer of the redox mediator. For example, the cross linking agent can be a polyepoxide (e.g., a polyethylene glycol diglycidylether (PEGDGE), ethylene glycol diglycidyl ether (EGDGE), resorcinol diglycidyl ether, 1,2,7,8-diepoxyoctane, Gly3), cyanuric chloride, N-hydroxysuccinimide, an imidoester, epichlorohydrin, or a combination thereof. In an aspect, the cross linking agent added to the polymer or copolymer is a polyethylene glycol diglycidylether (PEGDGE) of the following formula:

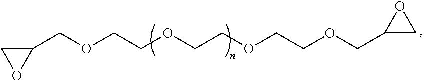

wherein n is an integer from 1 to about 50 (e.g., 1 to about 45, 1 to about 40, 1 to about 35, 1 to about 30, 1 to about 25, about 5 to about 50, about 5 to about 45, about 5 to about 40, about 5 to about 35, or about 5 to about 30).

In a particular example, the PEGDGE is PEGDGE200, PEGDGE400 (n is 10), PEGDGE500, PEGDGE600, PEGDGE1000, or PEGDGE2000, in which the number denotes the average molecular weight (Mₙ). In an aspect, the crosslinking agent is PEGDGE400.

In an aspect, at least a portion of the analyte (e.g., glucose) present in the sensing layer is non-leachably attached to the redox mediator. In some aspects, the enzyme is covalently attached to the polymer portion of the redox mediator. Covalent bonding of the enzyme to the redox material (e.g., polymer) can take place via the crosslinking agent, as described herein, and a reactive site on the enzyme. Thus, the enzyme is electronically “wired” to a working electrode through the redox material. In an aspect, a hydrogel is formed upon crosslinking the enzyme and its wire on electrodes. In another aspect, at least a portion of the enzyme can diffuse into the hydrogel and becomes attached but not necessarily covalently bonded to the polymer.

### Interference Domain

In one or more aspects, the analyte sensor of the present disclosure, e.g., an implantable portion 200, can optionally include an interference domain. In some aspects, the interference domain can include a polymer domain that restricts the flow of one or more interferants, e.g., to the surface of the working electrode. In some aspects, the interference domain can function as a molecular sieve that allows analytes and other substances that are to be measured by the working electrode to pass through, while preventing or reducing passage of other substances, such as interferents. In some aspects, the interferents can affect the signal obtained at the working electrode. Non-limiting examples of interferents can include acetaminophen, ascorbate, ascorbic acid, bilirubin, cholesterol, creatinine, dopamine, ephedrine, ibuprofen, L-dopa, methyldopa, salicylate, tetracycline, tolazamide, tolbutamide, triglycerides, urea, and uric acid.

In some aspects, the interference domain can be located between the working electrode and one or more sensing spots. In some aspects, non-limiting examples of polymers that can be used in forming the interference domain include polyurethanes, polymers having pendant ionic groups, and polymers having controlled or selected pore size. In some aspects, the interference domain can be formed from one or more cellulosic derivatives.

Non-limiting examples of cellulosic derivatives include polymers, such as cellulose acetate, cellulose acetate butyrate, 2-hydroxyethyl cellulose, cellulose acetate phthalate, cellulose acetate propionate, cellulose acetate trimellitate, and the like.

In one or more aspects, the interference domain can be part of the diffusion-limiting membrane and not a separate membrane. In some aspects, the interference domain can be located between the one or more sensing spots and the diffusion-limiting membrane.

In one or more aspects, the interference domain can include a thin, hydrophobic membrane that is non-swellable and restricts diffusion of high molecular weight species. For example, but not by way of limitation, the interference domain can be permeable to relatively low molecular weight substances, such as hydrogen peroxide, while restricting the passage of higher molecular weight substances, such as ketones, glucose, acetaminophen and/or ascorbic acid.

### Polymer Membrane

The polymer membrane 214 comprises a polymer matrix that overcoats at least a portion of the sensing layer 208 and functions as a diffusion-limiting membrane and/or to improve biocompatibility (FIG. 2).

A diffusion-limiting membrane can act as a diffusion-limiting barrier to reduce the rate of mass transport of the analyte (e.g., glucose) when the sensor is in use. For example, but not by way of limitation, limiting access of an analyte (e.g., glucose) to the sensing spot with a diffusion-limiting membrane can aid in avoiding sensor overload (saturation), thereby improving detection performance and accuracy. In one or more aspects, the diffusion-limiting layer can limit the flux of an analyte to the electrode in an electrochemical sensor so that the sensor is linearly responsive over a large range of analyte concentrations.

In one or more aspects, the polymer membrane can be homogeneous and can be single-component (e.g., contain a single membrane polymer). In some aspects, the polymer membrane can be multi-component (e.g., contain two or more different membrane polymers). In some aspects, the multi-component membrane can be present as a bilayer membrane or as a homogeneous admixture of two or more membrane polymers. A homogeneous admixture can be deposited by combining the two or more membrane polymers in a solution and then depositing the solution upon a working electrode, e.g., by dip coating.

In one or more aspects, the diffusion-limiting membrane can include two or more layers, e.g., a bilayer or trilayer membrane. In some aspects, each layer can include a different polymer or the same polymer at different concentrations or thicknesses.

In one or more aspects, a diffusion-limiting membrane can include a polymer matrix containing one or more heterocyclic nitrogen groups. In some aspects, a diffusion-limiting membrane can include a polyvinylpyridine-based polymer. Non-limiting examples of polyvinylpyridine-based polymers are disclosed in U.S. Patent Publication No. 2003/0042137, the disclosure of which is incorporated by reference herein in its entirety. In some aspects, the polyvinylpyridine-based polymer has a molecular weight from about 50 kD to about 500 kD, e.g., from about 50 kD to about 200 kD.

In one or more aspects, a diffusion-limiting membrane can include a polymer matrix formed from a polymer selected from a polyvinylpyridine (e.g., poly(4-vinylpyridine) or poly(2-vinylpyridine)), a polyvinylimidazole, a polyvinylpyridine copolymer (e.g., a copolymer of vinylpyridine and styrene), a polyacrylate, a polyurethane, a polyether urethane, a silicone, a polytetrafluoroethylene, a polyethylene-co-tetrafluoroethylene, a polyolefin, a polyester, a polycarbonate, a biostable polytetrafluoroethylene, homopolymers, copolymers or terpolymers of polyurethanes, a polypropylene, a polyvinylchloride, a polyvinylidene difluoride, a polybutylene terephthalate, a polymethylmethacrylate, a polyether ether ketone, cellulosic polymers, polysulfones, and any combination thereof, such as blends or block copolymers thereof, including, for example, di-block, tri-block, alternating, random and graft copolymers. In an aspect, the polymer matrix in the membrane can be formed from at least one polymer selected from the group consisting of a polyvinylpyridine-based polymer, a polyvinylimidazole-based polymer, a polyacrylate, a poly(amino acid), a polyurethane, a polyether urethane, a silicone, and any combination thereof. In some aspects, the polymer matrix comprises a polyvinylpyridine-based polymer, a polyvinylimidazole-based polymer, or a combination thereof.

In one or more aspects, the polymer matrix in the polymer membrane can be formed from a polyvinylpyridine (e.g., poly(4-vinylpyridine) and/or poly(2-vinylpyridine)). In some aspects, the polymer matrix can be formed from poly(4-vinylpyridine). In some aspects, the polymer matrix can be formed from a copolymer of vinylpyridine and styrene. In some aspects, the polymer matrix can be formed from a polyvinylpyridine-co-styrene copolymer. For example, but not by way of limitation, a polyvinylpyridine-co-styrene copolymer can include a polyvinylpyridine-co-styrene copolymer in which a portion of the pyridine nitrogen atoms are functionalized with a non-crosslinked polyethylene glycol tail and a portion of the pyridine nitrogen atoms are functionalized with an alkylsulfonic acid group, e.g., a propylsulfonic acid. In some aspects, a derivatized polyvinylpyridine-co-styrene copolymer for use as the polymer matrix can be the 10Q5 polymer, as described in U.S. Pat. No. 8,761,857, the disclosure of which is incorporated by reference herein in its entirety.

In an aspect, a suitable copolymer of vinylpyridine and styrene can have a styrene content (e.g., amount) in a range of about 0.01% to about 50% mole percent, or from about 0.05% to about 45% mole percent, or from about 0.1% to about 40% mole percent, or from about 0.5% to about 35% mole percent, or from about 1% to about 30% mole percent, or from about 2% to about 25% mole percent, or from about 5% to about 20% mole percent. In some aspects, a copolymer of vinylpyridine and styrene can include a styrene content (e.g., amount) in a range of about 2% to about 25% mole percent. Substituted styrene can be used similarly and in similar amounts.

In an aspect, a suitable copolymer of vinylpyridine and styrene can have a weight average molecular weight of about 1 kD or more, or about 5 kD or more, or about 10 kD or more, or about 15 kD or more, or about 20 kD or more, or about 25 kD or more, or about 30 kD or more, or about 40 kD or more, or about 50 kD or more, or about 75 kD or more, or about 90 kD or more, about 100 kD or more, or about 110 kD or more. In non-limiting examples, a suitable copolymer of vinylpyridine and styrene can have a weight average molecular weight in a range of about 5 kD to about 150 kD, or from about 10 kD to about 125 kD, or from about 15 kD to about 100 kD, or from about 20 kD to about 80 kD, or from about 25 kD to about 75 kD, or from about 30 kD to about 60 kD. In some aspects, a copolymer of vinylpyridine and styrene can have a weight average molecular weight in a range of about 10 kD to about 125 kD.

In one or more aspects, the analyte sensor of the present disclosure can include an implantable portion, e.g., 200, including: a working electrode including a sensing layer 208, such as working electrode 204, a counter electrode, such as counter electrode 206, and a reference electrode, such as reference electrode 212; and a polymer membrane 214 comprising an antimicrobial agent (FIG. 2).

Biofilm formation and one or more types of bacteria colony-forming units (CFUs) can be associated with analyte sensors exhibiting late signal attenuation. In an aspect, the present disclosure provides a multi-tiered technique to reduce immune cell infiltration to an implant site through the use of antibiotics to inhibit microorganisms and eliminate an infection-related immune response from a host, which can lead to high immune cell density and tissue encapsulations. Thus, in an aspect, the polymer membrane can include a polymer matrix and one or more antimicrobial agents. The one or more antimicrobial agents can be released in close proximity to an analyte sensor in vivo. The incorporation of an antimicrobial agent within the analyte sensor itself or the delivery of the antimicrobial agent in close proximity to the sensor at its in vivo location allows targeted delivery of the antimicrobial agent to the tissue around (e.g., surrounding) the implantation site and the analyte sensor.

In one or more of these aspects in which the polymer membrane comprises at least one antimicrobial agent, the polymer matrix can be formed from a polymer selected from the group consisting of a polyvinylpyridine-based polymer, a polyvinylimidazole-based polymer, a poly(amino acid), a polyacrylate, a polyurethane, a polyether urethane, a silicone, and any combination thereof.

In one or more of these aspects, the polymer matrix can comprise a polyvinylpyridine-based copolymer, a polyvinylimidazole-based copolymer, or a combinations thereof. In one or more aspects, the copolymer can be an alternating copolymer, a random copolymer, a block copolymer, or a graft copolymer. In an aspect, the copolymer can be an alternating copolymer; in another aspect, the copolymer can be a random copolymer; in yet another aspect, the copolymer can be a block copolymer.

In some of these aspects, the polyvinylimidazole-based copolymer can be a copolymer of vinylimidazole and styrene or a substituted derivative thereof. In some aspects, the polyvinylimidazole-based copolymer can be an optionally substituted polyvinylpyridine-co-polystyrene polymer. In some aspects, the polyvinylimidazole-co-polystyrene polymer can be a poly(N-vinylimidazole)-co-polystyrene polymer, a poly(1-vinylimidazole)-co-polystyrene polymer, or a substituted derivative thereof.

In one or more of these aspects, the polymer matrix can include at least one non-crosslinked polyvinylpyridine-based copolymer and at least one crosslinked polyvinylpyridine-based copolymer. In one or more aspects, a weight ratio of the least one non-crosslinked polyvinylpyridine-based copolymer and the at least one crosslinked polyvinylpyridine-based copolymer is can be in a range of about 9:1 to about 1:9, about 9:1 to about 2:8, about 9:1 to about 3:7, about 9:1 to about 4:6, about 9:1 to about 5:5, about 9:1 to about 6:4, about 9:1 to about 7:3, about 9:1 to about 8:2, about 8:2 to about 9:1, about 7:3 to about 9:1, about 6:4 to about 9:1, about 5:5 to about 9:1, about 4:6 to about 9:1, about 3:7 to about 9:1, or about 2:8 to about 9:1. In some aspects, the weight ratio of the least one non-crosslinked polyvinylpyridine-based copolymer and the at least one crosslinked polyvinylpyridine-based copolymer can be about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, or about 1:9, or in a range defined between any two of the foregoing values, for example in a range between about 6:1 and about 4:1.

In one or more of these aspects, the polymer matrix can include at least one non-crosslinked polyvinylimidazole-based copolymer and at least one crosslinked polyvinylimidazole-based copolymer. In one or more aspects, a weight ratio of the least one non-crosslinked polyvinylimidazole-based copolymer and the at least one crosslinked polyvinylimidazole-based copolymer is can be in a range of about 9:1 to about 1:9, about 9:1 to about 2:8, about 9:1 to about 3:7, about 9:1 to about 4:6, about 9:1 to about 5:5, about 9:1 to about 6:4, about 9:1 to about 7:3, about 9:1 to about 8:2, about 8:2 to about 9:1, about 7:3 to about 9:1, about 6:4 to about 9:1, about 5:5 to about 9:1, about 4:6 to about 9:1, about 3:7 to about 9:1, or about 2:8 to about 9:1. In some aspects, the weight ratio of the least one non-crosslinked polyvinylimidazole-based copolymer and the at least one crosslinked polyvinylimidazole-based copolymer can be about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, or about 1:9, or in a range defined between any two of the foregoing values, for example in a range between about 6:1 and about 4:1.

In one or more of these aspects, the polyvinylpyridine-based copolymer can be a polyvinylpyridine-co-polystyrene polymer or a substituted derivative thereof. In one or more aspects, the polyvinylpyridine-co-polystyrene polymer can include about 1-about 50 mer % of styrene units. In some aspects, the polyvinylpyridine-co-polystyrene polymer can include about 1-about 30 mer %, about 1%-25%, about 1%-20%, about 1%-15%, about 1%-10%, about 2%-10%, about 3%-10%, about 4%-10%, about 5%-10%, about 6%-10%, about 7%-10%, about 8%-10%, or about 9%-10%, or with any range defined between any two of the foregoing values, such as in a range of about 7%-15%, of styrene units.

In one or more of these aspects, the polyvinylpyridine-co-polystyrene polymer can be poly(4-vinylpyridine-co-styrene), poly (2-vinylpyridine-co-styrene), or a substituted derivative thereof.

In one or more of these aspects, the polyvinylpyridine-co-polystyrene polymer can include charged pyridine moieties.

In one or more of these aspects, the polyvinylpyridine-co-polystyrene polymer can include a portion (e.g., about 10% of the total number of pyridine nitrogen atoms) of pyridine nitrogen atoms that are functionalized with a non-crosslinked polyethylene glycol tail and a portion (e.g., about 5% of the total number of pyridine nitrogen atoms) of pyridine nitrogen atoms that are functionalized with an alkylsulfonic acid group. In some aspects, a derivatized polyvinylpyridine-co-styrene copolymer for use as a coating polymer can be the 10Q5 polymer as described in U.S. Pat. No. 8,761,857, the disclosure of which is incorporated by reference herein in its entirety.

In one or more of these aspects, a weight averaged molecular weight of the polymer matrix can be in a range of about 1 kD-1,000 kD, about 1 kD-800 kD, about 1 kD-600 kD, about 1 kD-400 kD, about 1 kD-200 kD, about 1 kD-100 kD, about 2 kD-100 kD, about 5 kD-100 kD, about 10 kD-100 kD, about 20 kD-100 kD, about 30 kD-100 kD, about 40 kD-100 kD, about 50 kD-100 kD, about 60 kD-100 kD, about 70 kD-100 kD, about 80 kD-100 kD, about 100 kD-200 kD, about 100 kD-300 kD, or about 100 kD-400 kD, or with any range defined between any two of the foregoing values, such as in a range of about 10 kD-50 kD. In some aspects, a weight average molecular weight of the polymer matrix is in a range of about 100 kD-250 kD.

In one or more of these aspects, a weight averaged molecular weight of the polyvinylpyridine-based polymer can be in a range of about 1 kD-1,000 kD, about 1 kD-800 kD, about 1 kD-600 kD, about 1 kD-400 kD, about 1 kD-200 kD, about 1 kD-100 kD, about 2 kD-100 kD, about 5 kD-100 kD, about 10 kD-100 kD, about 20 kD-100 kD, about 30 kD-100 kD, about 40 kD-100 kD, about 50 kD-100 kD, about 60 kD-100 kD, about 70 kD-100 kD, about 80 kD-100 kD, about 100 kD-200 kD, about 100 kD-300 kD, or about 100 kD-400 kD, or with any range defined between any two of the foregoing values, such as in a range of about 10 kD-50 kD. In some aspects, a weight average molecular weight of the polyvinylpyridine-based polymer is in a range of about 100 kD-250 kD.

In one or more of these aspects, a weight averaged molecular weight of the polyvinylpyridine-co-polystyrene-based polymer can be in a range of about 1 kD-1,000 kD, about 1 kD-800 kD, about 1 kD-600 kD, about 1 kD-400 kD, about 1 kD-200 kD, about 1 kD-100 kD, about 2 kD-100 kD, about 5 kD-100 kD, about 10 kD-100 kD, about 20 kD-100 kD, about 30 kD-100 kD, about 40 kD-100 kD, about 50 kD-100 kD, about 60 kD-100 kD, about 70 kD-100 kD, about 80 kD-100 kD, about 100 kD-200 kD, about 100 kD-300 kD, or about 100 kD-400 kD, or with any range defined between any two of the foregoing values, such as in a range of about 10 kD-50 kD. In some aspects, a weight average molecular weight of the polyvinylpyridine-based polymer is in a range of about 100 kD-250 kD.

In one or more of these aspects, the polymer matrix in the polymer membrane can further be formed with a crosslinker including two or more crosslinkable groups. In one or more aspects, the crosslinker that is added to the polymer or copolymer can include polyepoxides, carbodiimide, cyanuric chloride, triglycidyl glycerol, N-hydroxysuccinimide, imidoesters, epichlorohydrin, amine-containing compounds, or any combination thereof.

In one or more aspects, the crosslinker that is added to the polymer or copolymer be a glycidyl ether crosslinker. In one or more aspects, the crosslinker can be a diglycidyl- or triglycidyl-functional ether. In one or more aspects, the crosslinker can be polyethylene glycol (PEG) diglycidyl ether. In one or more aspects, the crosslinker can be selected from the group consisting of diglycidyl-PEG (200-1000), glycerol triglycidyl ether, and a combination thereof. In one or more aspects, the crosslinker can be a diglycidyl-PEG (200-1000) with a molecular weight of 200 g/mol-1000 g/mol. The term “diglycidyl-PEG” used in the present disclosure refers to polyethylene glycol diglycidyl ether. In one or more aspects, the crosslinker can be selected from the group consisting of diglycidyl-PEG 200, diglycidyl-PEG 400, diglycidyl-PEG 1000, glycerol triglycidyl ether, and any combination thereof. In one aspect, the crosslinker can be diglycidyl-PEG 200. In one aspect, the crosslinker can be diglycidyl-PEG 400. In one aspect, the crosslinker can be diglycidyl-PEG 1000. In one or more aspects, the crosslinker can be triglycidyl glycerol.

In some aspects, of the crosslink density of the crosslinked polymer or copolymer can be in a range of about 1% to about 50% (e.g., about 1% to about 40%, about 1% to about 35%, about 1% to about 30%, about 1% to about 25%, about 1% to about 20%, about 1% to about 15%, about 1% to about 10%, about 1% to about 8%, or about 1% to about 5%). In some aspects, the crosslink density of the crosslinked polymer or copolymer can be in a range of about 1% to about 30%. Crosslink density can be measured using any suitable method, such as ASTM D2765 (updated Dec. 27, 2016).

In one or more aspects, the antimicrobial agent in the polymer membrane can be a therapeutic agent that is effective at reducing, minimizing, preventing, and/or inhibiting a microorganism infection around (e.g., surrounding) a sensor implantation site and the analyte sensor, to prevent and/or reduce early and/or late signal attenuation. In one or more aspects, the antimicrobial agent can be an antibiotic, an anti-fungal agent, an anti-infective agent, or a combination thereof. In some aspects, the antimicrobial agent can be an antibiotic, an anti-fungal agent, an anti-infective agent, or a combination thereof and is not a metal or a metal salt (e.g., AgCl).

In one or more aspects, the antimicrobial agent can inhibit, slow, and/or reduce the growth and colony formation of bacterium such as Staphylococcus epidermidis (s. epidermidis), Staphylococcus aureus subsp. aureus strain (UAMS-1), methicillin-resistant Staphylococcus aureus (MIRSA), Enterococcus faecalis (E. faecalis), Cutibacterium acnes (C. acnes), Streptococcus pyogenes (S. pyogenes), or any combination thereof.

In some aspects, the antimicrobial agent can comprise at least one (e.g., 1, 2, 3, or 4, etc.) antibiotic. In an aspect, the antibiotic can comprise a mixture of antibiotics in order to minimize acquired antibiotic resistance in association with sensor site infections.

In one or more aspects, the antimicrobial agent can be an antibiotic that does not interfere with sensor functionality or only minimally interferes with sensor functionality (collectively “minimally interfering antibiotics”). In some aspects, the minimally interfering antibiotic can be amoxicillin, ampicillin, aminoglycosides, azithromycin, aztreonam, aclarubicin, actinomycin D, actinoplanone, adriamycin, aeroplysinin-1, amrubicin, anthracycline, azinomycin-A, bisucaberin, bleomycin sulfate, bryostatin-1, cefepime, cefixime, ceftriaxone, cephalosporin C, cephazolin, cephamandol, chloramphenicol, ciprofloxacin, clindamycin, calichemycin, chromoximycin, dactinomycin, daunorubicin, ditrisarubicin B, doxorubicin, doxorubicin-fibrinogen, doxycycline, erythromycin, imipenem, meropenem, metronidazole, minocycline, minocycline hydrochloride, netilmicin, rifampin, rifamycins, spectinomycin, a penicillin, streptomycin, tetracycline, tobramycin, trimethoprim, elsamicin-A, epirubicin, erbstatin, esorubicin, esperamicin-Al, esperamicin-Alb, fostriecin, glidobactin, gregatin-A, grincamycin, herbimycin, idarubicin, illudins, kazusamycin, kesarirhodins, menogaril, mitomycin, neoenactin, oxalysine, oxaunomycin, peplomycin, pilatin, pirarubicin, porothramycin, pyrindanycin A, rapamycin, everolimus, tacrolimus, sirolimus, deforolimus, ridaforolimus, temsirolimus, rhizoxin, rodorubicin, sibanomicin, siwenimycin, sorangicin-A, sparsomycin, talisomycin, terpentecin, thrazine, tricrozarin A, zorubicin, or any combination of any of the foregoing.

In some aspects, the antibiotic can comprise a tetracycline class antibiotic. Examples of suitable tetracycline class antibiotics include, e.g., tetracycline, doxycycline, minocycline, tigecycline, chlortetracycline, oxytetracycline, demeclocycline, lymecycline, methacycline, minocycline, rolitetracycline, eravacycline, sarecycline, omadacycline, aminomethylcycline, glycylcycline, azatetracycline, 6-thiatetracycline, 4-epi-anhydrochloratetracycline, fluorocycline, pentacycline, and salts thereof (e.g., an acid addition salt thereof). In some aspects, the tetracycline class antibiotic can be minocycline salt, such as minocycline hydrochloride. In some aspects, the polymer matrix (i.e., a dry, non-hydrated polymer matrix) can comprise about 0.1 to about 5 wt % minocycline hydrochloride.

In some aspects, the antibiotic can comprise a tetracycline class antibiotic in combination with an ansamycin class antibiotic, which is any antibiotic produced by strains of several Actinomycetes. The ansamycin class antibiotic can be a benzenoid ansamycin, an herbimycin ansamycin, or a combination of both. Examples of a suitable ansamycin class antibiotic include, e.g., a rifamycin, geldanamycin, herbimycin, macbecin, ansamitocin, maytansine, ansatrienin, cytotrienin, hydroxymycotrienin, mycotrienin, thiazinotrienomycin, trienomycin, halomicin, streptovaricin, ansathiazin, awamycin, damavaricin, kanglemycin, proansamycin, protorifamycin, protostreptovaricin, tolypomycin, actamycin, naphthomycin, naphthomycinol, naphthoquinomycin, rubradirin, protorubradirin, and salts thereof. In some aspects, the ansamycincan class antibiotic be a rifamycin (e.g., rifamycin A, rifamycin B, rifamycin C, rifamycin D, rifamycin E, rifamycin L, rifamycin SV, rifampin (also called rifampicin), rifabutin, rifapentine, rifalazil, rifaximin, and salts thereof. In some aspects, the antibiotic can include rifampin.

In one or more aspects, the antimicrobial agent can include minocycline hydrochloride, rifampin, or a combination thereof. In an aspect, the antibiotic can comprise a combination of minocycline hydrochloride and rifampin. In one or more aspects, minocycline hydrochloride and rifampin can be present in a weight ratio ranging from about 1:99 to about 99:1, about 10:90 to about 90:10, about 10:90 to about 80:20, about 10:90 to about 70:30, about 10:90 to about 60:40, about 10:90 to about 50:50, about 10:90 to about 40:60, about 10:90 to about 30:70, or about 10:90 to about 20:80. In some aspects, a weight ratio of minocycline hydrochloride to rifampin can be about 1:20, about 1:10, about 1:9, about 1:8, about 1:7, about 1:6, about 1:5, about 1:4, about 1:3, about 1:2, about 1:1, about 2:1, about 3:1, about 4:1, about 5:1, about 6:1, about 7:1, about 8:1, about 9:1, about 10:1, or about 20:1, or in a range defined between any two aforementioned values, for example, in a range of about 1:10 to about 10:1, about 1:7 to about 1:1, or about 1:6 to about 1:4. In an another aspect, the weight ratio of minocycline hydrochloride to rifampin can be in a range of about 1:10 to about 10:1 or about 1:6 to about 1:4.

In an aspect, the antibiotic can be present in a total amount ranging from about 0.1 wt % to about 40 wt % based on a total weight of the polymer matrix (i.e., a dry, non-hydrated polymer matrix). For example, and in some aspects, the total amount of the antibiotic can be present in a range of about 0.1 wt % to about 35 wt %, about 0.1 wt % to about 30 wt %, about 0.1 wt % to about 25 wt %, about 0.1 wt % to about 20 wt %, about 1 wt % to about 40 wt % about 1 wt % to about 35 wt %, about 1 wt % to about 30 wt %, about 1 wt % to about 25 wt %, about 1 wt % to about 20 wt %, about 2 wt % to about 40 wt % about 2 wt % to about 35 wt %, about 2 wt % to about 30 wt %, about 2 wt % to about 25 wt %, about 2 wt % to about 20 wt %, about 5 wt % to about 40 wt % about 5 wt % to about 35 wt %, about 5 wt % to about 30 wt %, about 5 wt % to about 25 wt %, or about 5 wt % to about 20 wt %, based on the total weight of the polymer matrix (i.e., a dry, non-hydrated polymer matrix). In other aspects, the total amount of the antibiotic can be in a range of about 5 wt % to about 20 wt % based on the total weight of the polymer matrix (i.e., a dry, non-hydrated polymer matrix).

In one or more aspects, the polymer membrane can include from about 0.0005 milligrams (mg) to about 0.2 mg of the antimicrobial agent or any values in between. In some aspects, the polymer membrane can include about 0.0005 mg, about 0.001 mg, about 0.005 mg, about 0.01 mg, about 0.05 mg, about 0.1 mg, or about 0.2 mg of the antimicrobial agent. In some aspects, the polymer membrane can include from about 0.1 micrograms (μg) to about 20 μg of the antimicrobial agent. In some aspects, the polymer membrane can include from about 1 μg to about 100 μg of the antimicrobial agent, e.g., from about 1 μg to about 95 μg, from about 1 μg to about 90 μg, from about 1 μg to about 85 μg, from about 1 μg to about 80 μg, from about 1 μg to about 75 μg, from about 1 μg to about 70 μg, from about 1 μg to about 65 μg, from about 1 μg to about 60 μg, from about 1 μg to about 55 μg, from about 1 μg to about 50 μg, from about 1 μg to about 45 μg, from about 1 μg to about 40 μg, from about 1 μg to about 35 μg, from about 1 μg to about 30 μg, from about 1 μg to about 25 μg, from about 1 μg to about 20 μg, from about 1 μg to about 15 μg, from about 1 μg to about 14 μg, from about 1 μg to about 13 μg, from about 1 μg to about 12 μg, from about 1 μg to about 11 μg, from about 1 μg to about 10 μg, from about 1 μg to about 9 μg, from about 2 μg to about 100 μg, from about 3 μg to about 100 μg, from about 4 μg to about 100 μg, from about 5 μg to about 100 μg, from 5 about 6 μg to about 100 μg, from about 7 μg to about 100 μg, from about 8 μg to about 100 μg, from about 9 μg to about 100 μg, from about 10 μg to about 100 μg, from about 11 μg to about 100 μg, from about 12 μg to about 100 μg, from about 13 μg to about 100 μg, from about 14 μg to about 100 μg, from about 15 μg to about 100 μg, from about 16 μg to about 100 μg, from about 17 μg to about 100 μg, from about 18 μg to about 100 μg, from about 19 μg to about 100 μg, from about 20 μg to about 100 μg, from about 25 μg to about 100 μg, from about 30 μg to about 100 μg, from about 35 μg to about 100 μg, from about 40 μg to about 100 μg, from about 45 μg to about 100 μg, from about 50 μg to about 100 μg, from about 55 μg to about 100 μg, from about 60 μg to about 100 μg, from about 65 μg to about 100 μg, from about 70 μg to about 100 μg, from about 75 μg to about 100 μg, from about 80 μg to about 100 μg, from about 85 μg to about 100 μg, from about 90 μg to about 100 μg, from about 95 μg to about 100 μg, from about 5 μg to about 50 μg, from about 5 μg to about 45 μg, from about 5 μg to about 40 μg, from about 5 μg to about 35 μg, from about 5 μg to about 30 μg, from about 5 μg to about 25 μg, or from about 5 μg to about 20 μg. In some aspects, the polymer membrane can include from about 1 μg to about 20 μg of the antimicrobial agent. In some aspects, the polymer membrane can include from about 5 μg to about 20 μg of the antimicrobial agent. In some aspects, the polymer membrane can include from about 1 μg to about 30 μg of the antimicrobial agent. In some aspects, the polymer membrane can include from about 5 μg to about 30 μg of the antimicrobial agent.

In one or more aspects, the polymer membrane can continuously release the antimicrobial agent at a rate of about 0.01 μg/day to about 1 mg/day of the antimicrobial agent, or any values in between. In some aspects, the polymer membrane can continuously release the antimicrobial agent at a rate of about 0.1 μg/day, about 0.2 μg/day, about 0.3 μg/day, about 0.4 μg/day, about 0.5 μg/day, about 0.6 μg/day, about 0.7 μg/day, about 0.8 μg/day, about 0.9 μg/day, about 1 μg/day, about 2 μg/day, about 3 μg/day, about 4 μg/day, about 5 μg/day, about 6 μg/day, about 7 μg/day, about 8 μg/day, about 9 μg/day, about 10 μg/day, about 20 μg/day, about 30 μg/day, about 40 μg/day, about 50 μg/day, about 60 μg/day, about 70 g/day, about 80 μg/day, about 900 μg/day, about 100 μg/day, about 200 μg/day, about 300 μg/day, about 400 μg/day, about 500 μg/day, about 600 μg/day, about 700 μg/day, about 800 μg/day, about 900 μg/day, or about 1 mg/day of the antimicrobial agent, or any values in between. In some aspects, the polymer membrane can continuously release the antimicrobial agent at a rate of about 0.2 μg/day to about 5 μg/day of the antimicrobial agent. In some aspects, the polymer membrane can continuously release the antimicrobial agent at a rate of about 0.2 μg/day to about 2 μg/day of the antimicrobial agent. In some aspects, the polymer membrane can continuously release the antimicrobial agent at a rate of about 0.2 μg/day to about 1 μg/day of the antimicrobial agent. In some aspects, the polymer membrane can continuously release the therapeutic agent at a set or predetermined drug delivery rate to achieve desirable therapeutic results such as reducing, minimizing, reducing, preventing, and/or inhibiting the antimicrobial infection.

In an aspect, the polymer membrane can provide improved sensor longevity. For example, the polymer membrane can continuously release the antimicrobial agent at a set or predetermined drug delivery rate for at least 1 day, for at least 2 days, for at least 3 days, for at least 4 days, for at least 5 days, for at least 6 days, for at least 7 days, for at least 8 days, for at least 9 days, for at least 10 days, for at least 11 days, for at least 12 days, for at least 13 days, for at least 14 days, for at least 15 days, for at least 16 days, for at least 17 days, for at least 18 days, for at least 19 days, for at least 20 days, for at least 21 days, for at least 22 days, for at least 23 days, for at least 24 days, for at least 25 days, for at least 26 days, for at least 27 days, for at least 28 days, for at least 29 days, or for at least 30 days. In some aspects, polymer membrane can continuously release the antimicrobial agent at a set or predetermined rate for a set or predetermined number of days such as for at least 30 days.

In one or more aspects, the polymer membrane can release the antimicrobial agent in a bolus at a set or predetermined delivery time.

In one or more aspects, the polymer membrane can have a thickness (e.g., dry thickness) in a range of about 0.1 μm to about 1,000 μm, e.g., from about 1 μm to and about 500 μm, about 10 μm to about 500 μm, about 10 μm to about 400 μm, about 10 μm to about 300 μm, about 10 μm to about 200 μm, or about 10 μm to about 100 μm. In some aspects, the polymer membrane can have a thickness in a range of about 1 μm to about 500 μm. In some aspects, the polymer membrane can have a thickness in a range of about 1 μm to about 400 μm. In some aspects, the polymer membrane can have a thickness in a range of about 1 μm to about 300 μm. In some aspects, the polymer membrane can have a thickness in a range of about 1 μm to about 200 μm. In some aspects, the polymer membrane can have a thickness in a range of about 10 μm to about 200 μm. In some aspects, the polymer membrane can have a thickness in a range of about 10 μm to about 300 μm. In some aspects, the polymer membrane can have a thickness in a range of about 50 μm to about 300 μm.

In one or more aspects, the polymer membrane can have a total mass (e.g., dry mass) in a range of about 1 μg to about 1,000 μg, e.g., from about 10 μg to about 1000 μg, about 20 μg to about 1000 μg, about 30 μg to about 1000 μg, about 40 μg to about 1000 μg, about 10 μg to about 900 μg, about 10 μg to about 800 μg, about 10 μg to about 700 μg, about 10 ag to about 600 μg, about 10 μg to about 500 μg, about 10 μg to about 400 μg, about 10 μg to about 300 μg, about 10 μg to about 200 μg, about 10 μg to about 100 μg, about 10 μg to about 90 μg, about 10 μg to about 80 μg, about 10 μg to about 70 μg, about 10 μg to about 60 μg, or about 10 μg to about 50 μg. In some aspects, the polymer membrane can have a total mass (e.g., dry mass) in a range of about 1 μg to about 500 μg. In some aspects, the polymer membrane can have a total mass (e.g., dry mass) in a range of about 25 μg to about 500 μg. In some aspects, the polymer membrane can have a total mass (e.g., dry mass) in a range of about 50 μg to about 300 μg. In some aspects, the polymer membrane can have total mass (e.g., dry mass) in a range of about 50 μg to about 250 μg. In some aspects, the polymer membrane can have a total mass (e.g., dry mass) in a range of about 80 μg to about 200 μg. In some aspects, the polymer membrane can have a total mass (e.g., dry mass) in a range of about 100 μg to about 250 μg. In some aspects, the polymer membrane can have a total mass (e.g., dry mass) in a range of about 100 μg to about 200 μg.

In one or more aspects, the polymer membrane can be a single layer or include a plurality of layers. In an aspect, the polymer membrane can be present with an outer, middle/interior, and/or inner layer. In an aspect, at least one antimicrobial agent (e.g., antibiotic) can be present in the outer layer, one or more middle/interior layers, or a combination of these layers. In an aspect, at least one antimicrobial agent (e.g., antibiotic) can be present in the outer layer of the polymer membrane. In an aspect, at least one antimicrobial agent (e.g., antibiotic) can be present in one or more of the middle/interior layers of the polymer membrane.

In one or more aspects, the analyte sensor can be configured to detect glucose.

In one or more aspects, the analyte sensor can be a dermal sensor.

In one or more aspects, the analyte sensor can be a subcutaneous analyte sensor such as a subcutaneously implanted biosensor. In one or more aspects, the analyte sensor can be a subcutaneous analyte sensor implanted in an infected site or wound bed.

In one or more aspects, the analyte sensor can be an intravenous sensor such as intravenously implanted sensor.

In one or more aspects, the polymer membrane can be a diffusion-limiting membrane. In one or more aspects, the diffusion-limiting membrane can be a glucose-limiting membrane.

In one or more aspects, the polymer membrane comprises a hydrogel coating disposed on (e.g., overcoating) the polymer membrane. In one or more aspects, the presence of a hydrogel overcoating the polymer membrane can prevent and/or reduce early and/or late signal attenuation.

In one or more aspects, a hydrogel coating, as described herein, is disposed below the polymer membrane (e.g., below layer 214 in FIG. 2). In one or more aspects, a hydrogel coating, as described herein, can be used in lieu of (i.e., in the absence of) the polymer membrane in the analyte sensor. For example, layer 214 can be the hydrogel coating.

In one or more aspects, the hydrogel coating can be formed from (i) a polymer selected from poly(acrylic acid), poly-(α,β)-DL-aspartic acid, poly-L-glutamic acid, a salt form thereof, and a combination thereof and (ii) a crosslinker to crosslink the polymer to form a hydrogel polymer matrix. In one or more aspects, the polymer can be poly(acrylic acid), e.g., of Formula A, a poly-(α,β)-DL-aspartic acid of Formula B, poly-L-glutamic acid of Formula C, a salt form of any of the foregoing, or any combination thereof.

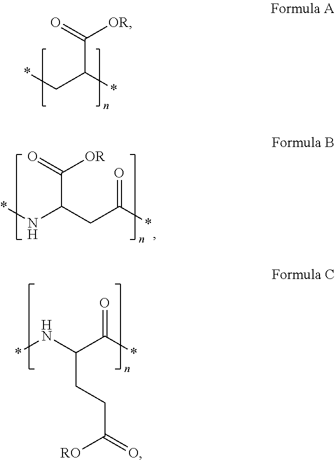

wherein R is H or a cation (e.g., a monovalent cation) to balance the charge. Suitable cations include, e.g., Group I cations (e.g., lithium, sodium, potassium), Group II cations (e.g., magnesium, calcium), tetralkylammonium, and ammonium. In some aspects of Formula A, B, or C, R is H. In some aspects of Formula A, B, or C, R is a Group I cation, such as a sodium cation.

In one or more aspects, the polymer used to form the hydrogel coating can be poly(acrylic acid), a salt form thereof, or a combination thereof. In one or more aspects, the poly(acrylic acid), the salt form thereof, or both can have a weight average molecular weight ranging from about 1 kD-1,000 kD, about 1 kD-900 kD, about 1 kD-800 kD, about 1 kD-700 kD, about 1 kD-600 kD, about 1 kD-500 kD, about 1 kD-400 kD, about 1 kD-300 kD, about 1 kD-200 kD, about 10 kD-200 kD, about 20 kD-200 kD, about 30 kD-200 kD, about 40 kD-200 kD, about 50 kD-200 kD, about 60 kD-200 kD, about 70 kD-200 kD, about 80 kD-200 kD, about 90 kD-200 kD, about 100 kD-200 kD, about 100 kD-300 kD, or about 100 kD-500 kD, or in any range defined between any two of the foregoing values, such as in a range of about 50 kD-200 kD. In some aspects, the weight average molecular weight of the poly(acrylic acid) is in a range of about 80 kD-150 kD.

In one or more aspects, the polymer used to form the hydrogel coating be poly-(α,β)-DL-aspartic acid, a salt form thereof, or a combination thereof. In one or more aspects, the poly-(α,β)-DL-aspartic acid, the salt form thereof, or both can have a weight average molecular weight in a range of about 1 kD-1,000 kD, about 1 kD-500 kD, about 1 kD-100 kD, about 1 kD-80 kD, about 1 kD-60 kD, about 1 kD-40 kD, about 1 kD-20 kD, about 1 kD-18 kD, about 1 kD-16 kD, about 1 kD-14 kD, about 1 kD-12 kD, about 1 kD-11 kD, about 1 kD-10 kD, about 1 kD-9 kD, about 1 kD-8 kD, about 1 kD-7 kD, about 1 kD-6 kD, about 1 kD-5 kD, about 1 kD-4 kD, about 1 kD-3 kD, or about 1 kD-2 kD, or with any range defined between any two of the foregoing values, such as in a range of about 2 kD-20 kD. In some aspects, the weight average molecular weight of the poly-(α,β)-DL-aspartic acid can be in a range of about 1 kD-11 kD.

In one or more aspects, the polymer used to form the hydrogel coating be poly-L-glutamic acid, a salt form thereof, or a combination thereof. In one or more aspects, a weight averaged molecular weight of poly-L-glutamic acid, a salt form thereof, both can have a weight average molecular weight in a range of about 1 kD-1,000 kD, about 1 kD-500 kD, about 1 kD-100 kD, about 1 kD-80 kD, about 1 kD-60 kD, about 1 kD-40 kD, about 1 kD-20 kD, about 1 kD-18 kD, about 1 kD-16 kD, about 1 kD-14 kD, about 1 kD-12 kD, about 1 kD-11 kD, about 1 kD-10 kD, about 1 kD-9 kD, about 1 kD-8 kD, about 1 kD-7 kD, about 1 kD-6 kD, about 1 kD-5 kD, about 1 kD-4 kD, about 1 kD-3 kD, or about 1 kD-2 kD, or with any range defined between any two of the foregoing values, such as in a range of about 10 kD-200 kD. In some aspects, a weight average molecular weight of the poly-L-glutamic acid is in a range of about 15 kD-100 kD.

In one or more aspects, the crosslinker used to form the hydrogel coating can be as described herein. In some aspect, the crosslinker can comprise a polyfunctional aziridine. In one or more aspects, the crosslinker can be trimethylolpropane tris(2-methyl-1-aziridinepropionate):

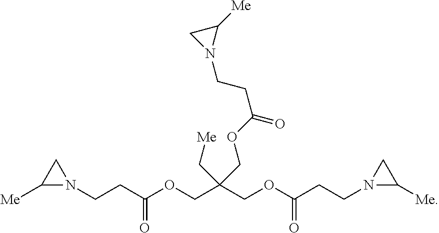 [SMILES: CCC(COC(=O)CCN1CC1C)(COC(=O)CCN1CC1C)COC(=O)CCN1CC1C]

In one or more aspects, the crosslinker can be present in the hydrogel coating in an amount ranging from about 0.1 wt % to about 10 wt %, for example, about 0.2 wt % to 10 wt %, about 0.3 wt % to 10 wt %, about 0.4 wt % to 10 wt %, about 0.5 wt % to 10 wt %, about 0.6 wt % to 10 wt %, about 0.7 wt % to 10 wt %, about 0.8 wt % to 10 wt %, about 0.9 wt % to 10 wt %, about 1 wt % to 10 wt %, about 1 wt % to 9 wt %, about 1 wt % to 8 wt %, about 1 wt % to 7 wt %, about 1 wt % to 6 wt %, about 1 wt % to 5 wt %, about 1 wt % to 4 wt %, about 1 wt % to 3 wt %, about 1 wt % to 2 wt %, or within any range defined between any two of the foregoing values, such as in a range of about 2 wt % to 4 wt %, based on the total weight of the hydrogel coating (i.e., dry mass).

In one or more aspects of this analyte sensor, the hydrogel coating can comprise an antimicrobial agent, such as an antibiotic, an anti-fungal agent, an anti-infective agent, or any combination thereof, as described herein. In some aspects, the hydrogel coating can comprise an antimicrobial agent comprising (i) a metal selected from the group consisting of silver, copper, zinc, gold, platinum, palladium, titanium, an alloy of any of the foregoing, and mixtures of any of the foregoing; and/or (ii) a corresponding metal salt thereof. In some aspects, the hydrogel coating can comprise an antimicrobial agent comprising silver, a silver salt, or a combination of both. In some aspects, the silver salt can be silver chloride, silver iodide, or a combination of both.

In one or more aspects, the hydrogel coating can comprise a fluoride ion compound as an antimicrobial agent. In one or more aspects, a fluoride ion is paired with an appropriate cation, such as an alkali metal cation, an alkaline earth metal cation, a transition metal cation, an ammonium cation, and any combination thereof, to form a fluoride ion compound. In one or more aspects, the fluoride ion compound can be provided by, or present as, sodium fluoride.

In one or more of these aspects, an amount of the fluoride ion compound (e.g., sodium fluoride) can be in a range of 0 wt % to about 70 wt %, for example, about 1 wt % to 60 wt %, about 1 wt % to 50 wt %, about 1 wt % to 40 wt %, about 5 wt % to 70 wt %, about 5 wt % to 60 wt %, about 5 wt % to 50 wt %, about 5 wt % to 40 wt %, about 10 wt % to 70 wt %, about 10 wt % to 60 wt %, about 10 wt % to 50 wt %, about 10 wt % to 40 wt %, about 15 wt % to 70 wt %, about 15 wt % to 60 wt %, about 15 wt % to 50 wt %, about 15 wt % to 40 wt %, about 20 wt % to 70 wt %, about 20 wt % to 60 wt %, about 20 wt % to 50 wt %, about 20 wt % to 40 wt %, based on the total weight of the hydrogel coating (i.e., dry mass). In one or more aspects, an amount of the fluoride ion (e.g., F⁻) can be in a range of 0 wt % to about 30 wt %, for example, about 1 wt % to 30 wt %, about 1 wt % to 20 wt %, about 1 wt % to 10 wt %, about 5 wt % to 30 wt %, about 5 wt % to 20 wt %, about 5 wt % to 10 wt %, about 10 wt % to 30 wt %, about 10 wt % to 20 wt %, about 15 wt % to 30 wt %, about 15 wt % to 20 wt %, or about 20 wt % to 30 wt %, based on the total weight of the hydrogel coating (i.e., dry mass).

For a transcutaneous analyte sensor, such as a Libre™ sensor (Abbott Diabetes Care, Alameda, CA), the sensor insertion site is exposed to an ambient environment during wear making it susceptible to possible bacterial infection that can affect sensor performance. With the use of printed or coated Ag/AgCl ink, AgCl is an Ag ion source, which can slowly, passively dissolve in water or a biological fluid. However, there is no control over Ag ion generation and release. The silver coating or components in their metallic form (e.g., Ag nanoparticles) are inert. Silver ion generation relies on the natural oxidation of silver, such that the rate of silver ion generation depends on pH, oxygen concentration, and/or other oxidant availability.

In an aspect, the present disclosure is directed toward active generation of a metal ion, such as Ag ion, via an electrochemical method. For example, a metal-containing layer can couple to a potentio/galvano-stat to actively generate metal ions, e.g., actively electrochemically generate metal ions. The metal ion generation rate, time, and duration can be controlled. In an example, silver in a silver-containing coating can be converted to Ag ion in an electrochemical reaction in a biological fluid, which typically contains relatively high concentrations of chloride ions, as follows:

Ag⁰+Cl⁻→AgCl+e⁻

The electrochemical production of Ag ions around the implantable portion provides an in situ antimicrobial agent to inhibit, slow, and/or reduce the growth and colony-formation of bacteria, thereby reducing or preventing late signal attenuation of the analyte sensor.

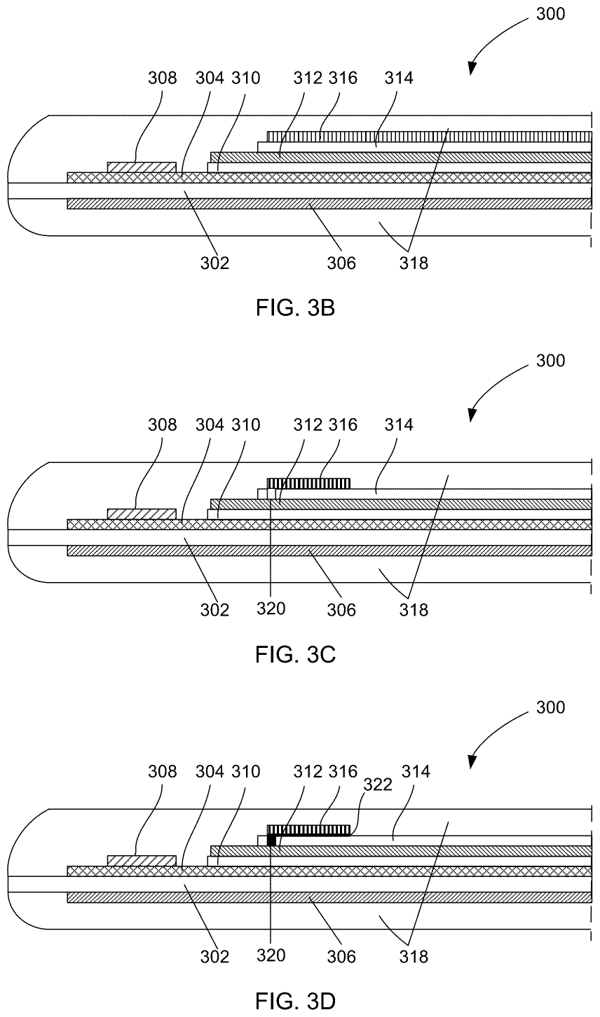

Accordingly, in some aspects, the analyte sensor comprises a metal-containing layer in electrochemical communication with the reference electrode and/or the counter electrode in addition to the presence of the antimicrobial agent and/or hydrogel coating. In one or more aspects, the metal-containing layer 316 can be in electrochemical communication with the counter electrode 306 and/or the reference electrode 312. Electrochemical communication with reference electrode 312 is shown in FIGS. 3C and 3D). In some embodiments, the metal-containing layer can be in electrochemical communication with the counter electrode. In some embodiments, the metal-containing layer can be in electrochemical communication with the reference electrode 312. In some embodiments, metal-containing layers can be in electrochemical communication with the counter electrode and the reference electrode. The metal-containing layer is capable of in situ formation of an antimicrobial agent comprising a metal ion through an electrochemical reaction.

In one or more aspects, the metal-containing layer can include a silver-containing material, a copper-containing material, a zinc-containing material, a gold-containing material, a platinum-containing material, a palladium-containing material, a titanium-containing material, or any combination thereof. In one or more aspects, the metal-containing layer can include: (i) a metal selected from the group consisting of silver, copper, zinc, gold, platinum, palladium, titanium, an alloy of any of the foregoing, and mixtures of any of the foregoing; and (ii) optionally a corresponding metal salt thereof. In some aspects, the metal-containing layer can include a metal selected from the group consisting of silver, copper, zinc, gold, platinum, palladium, titanium, an alloy of any of the foregoing, and mixtures of any of the foregoing. In other aspects, the metal-containing layer can include: (i) a metal selected from the group consisting of silver, copper, zinc, gold, platinum, palladium, titanium, an alloy of any of the foregoing, and mixtures of any of the foregoing; and (ii) a corresponding metal salt thereof.

In one or more aspects, the metal can be in a form of a film, a wire, or a particle. In one or more aspects, the metal-containing layer can include: (i) a silver film, silver wires, and/or silver particles; and (ii) a silver salt. In some aspects, the silver salt can be silver chloride.

In one or more aspects, the metal wire (e.g., silver wire) can have a diameter of about 1 nm to about 100 μm, for example, about 1 nm to about 80 μm, about 1 nm to about 50 μm, about 1 nm to about 40 μm, about 1 nm to about 30 μm, about 1 nm to about 20 μm, about 1 nm to about 10 μm, about 1 nm to about 1 μm, about 1 nm to about 800 nm, about 1 nm to about 600 nm, about 1 nm to about 400 nm, about 1 nm to about 200 nm, about 1 nm to about 100 nm, about 1 nm to about 50 nm, about 1 nm to about 20 nm, or about 1 nm to about 10 nm, and an aspect ratio of more than or equal to 2, more than or equal to 5, more than or equal to 10, more than or equal to 50, or more than or equal to 100. For example, in some aspects, the metal wire can have a diameter of about 1 nm to about 10 nm and an aspect ratio of about 50 to about 100.

In one or more aspects, the metal particles (e.g., silver particles) can have an average diameter or size of about 1 nm to about 100 μm, for example, about 1 nm to about 80 μm, about 1 nm to about 50 μm, about 1 nm to about 40 μm, about 1 nm to about 30 μm, about 1 nm to about 20 μm, about 1 nm to about 10 μm, about 1 nm to about 1 μm, about 1 nm to about 800 nm, about 1 nm to about 600 nm, about 1 nm to about 400 nm, about 1 nm to about 200 nm, about 1 nm to about 100 nm, about 1 nm to about 50 nm, about 1 nm to about 20 nm, or about 1 nm to about 10 nm.

In the present disclosure, when particles are spherical, or the cross-sections of metal wires are circular, the term “diameter” indicates a particle or wire diameter or an average particle or average wire diameter. When particles are non-spherical or the cross-sections of metal wires are not circular, the “diameter” indicates a major axis length or an average major axis length. The diameter (or size) of the particles/wires can be measured utilizing a scanning electron microscope or a (particle) size analyzer. As the (particle) size analyzer, for example, an LA-950 laser (particle) size analyzer (Horiba, Japan), can be used. When the size of the particles is measured utilizing a particle size analyzer, the average particle diameter (or size) is referred to as D50. D50 refers to the average diameter (or size) of particles whose cumulative volume corresponds to 50 vol % in the particle size distribution (e.g., cumulative distribution), and refers to the value of the particle size corresponding to 50% from the smallest particle when the total number of particles is 100% in the distribution curve accumulated in the order of the smallest particle size to the largest particle size.

In one or more aspects, the metal-containing layer can be a screen-printed silver coating including silver particles and a silver salt.

FIG. 3A illustrates a cross-sectional diagram of an implantable portion of an analyte sensor according to one or more aspects of the present disclosure. As shown in FIG. 3A, the analyte sensor can include an implantable portion 300 including: (i) a substrate 302; (ii) a working electrode 304 on substrate 302; (ii) a sensing layer 308 disposed upon a surface of the working electrode 304 for detecting an analyte; (iii) a counter electrode 306 on the substrate 302; (iv) a first dielectric (insulating) layer 310; (v) a reference electrode 312; (vi) a second dielectric (insulating) layer 314; (vii) a metal-containing layer 316 that is printed on the second dielectric (insulating) layer 314; and (vi) a membrane 318 overcoating at least the sensing layer 308, including an optional hydrogel coating (not shown) overcoating membrane 318. The metal-containing layer 316 can dissolve over time when in use and passively release the metal and/or a metal salt to provide antimicrobial activity. In some embodiments, the metal-containing layer 316 can be adjacent to the counter electrode 306 with a dielectric (insulating) layer disposed in between.

In some embodiments, the metal-containing layer 316 is printed the full length of the second dielectric (insulating) layer 314 (FIG. 3B).

FIG. 3C illustrates a cross-sectional diagram of an implantable portion of an analyte sensor according to one or more aspects of the present disclosure. As shown in FIG. 3C, the analyte sensor can include an implantable portion 300 including: (i) a substrate 302; (ii) a working electrode 304 on substrate 302; (ii) a sensing layer 308 disposed upon a surface of the working electrode 304 for detecting an analyte; (iii) a counter electrode 306 on the substrate 302; (iv) a first dielectric (insulating) layer 310; (v) a reference electrode 312; (vi) an optional second dielectric (insulating) layer 314; (vii) a metal-containing layer 316 that is in electrical communication with reference electrode 312 when either the dielectric (insulating) layer 314 is not present or through a via (hole) 320 in dielectric (insulating) layer 314; and (vi) a membrane 318 overcoating at least the sensing layer 308, including an optional hydrogel coating (not shown) overcoating the membrane 318. When an electric current is applied to the analyte sensor, the metal-containing layer 316 can actively release the metal and/or a metal salt to provide antimicrobial activity. In some embodiments, via (hole) 320 can be filled with the same material as the metal-containing layer 316.

FIG. 3D illustrates another embodiment of a cross-sectional diagram of an implantable portion of an analyte sensor according to one or more aspects of the present disclosure. As shown in FIG. 3D, the analyte sensor can include an implantable portion 300 including: (i) a substrate 302; (ii) a working electrode 304 on substrate 302; (ii) a sensing layer 308 disposed upon a surface of the working electrode 304 for detecting an analyte; (iii) a counter electrode 306 on the substrate 302; (iv) a first dielectric (insulating) layer 310; (v) a reference electrode 312; (vi) an optional second dielectric (insulating) layer 314; (vii) a metal-containing layer 316 that is in electrical communication with reference electrode 312 when either the dielectric (insulating) layer 314 is not present or through a carbon pad 322 that can fill the via (hole) 320 in dielectric (insulating) layer 314; and (vi) a membrane 318 overcoating at least the sensing layer 308, including an optional hydrogel coating (not shown) overcoating the membrane 318.

In one or more aspects, as shown in FIGS. 3A and 3B, the substrate 302 can be disposed between the working electrode 304 and the counter electrode 306. In one or more aspects, different from aspects shown in FIGS. 3A and 3B, the working electrode 304 and the counter electrode 306 can be located upon the same side of the substrate 302 with a dielectric (insulating) material interposed therebetween.

In an aspect, the sensing layer 308 can be disposed as at least one layer upon at least a portion of the working electrode 304. The sensing layer 308 can include an active area such as a sensing spot configured to detect an analyte through sensing chemistry.

In an aspect, the sensing layer 308 can include a plurality of sensing spots on the working electrode 304. The plurality of sensing spots can be responsive to different analytes and are laterally spaced apart from one another on the surface of the working electrode 304. In some aspects, at least some of the plurality of sensing spots can be responsive to a same analyte, and the rest can be responsive to different analytes. The membrane 318 can at least cover a portion of the plurality of sensing spots or all of the plurality of sensing spots. The composition of the membrane 318 can vary or be compositionally the same at the plurality of sensing spots. The plurality of sensing spots can be configured to detect their corresponding analytes at working electrode potentials that differ from one another.

In one or more aspects, the first dielectric layer 310 can be disposed between the working electrode 304 and the reference electrode 312. In one or more aspects, different from the configuration shown in FIGS. 3A and 3B, the reference electrode 312 can be the side of the counter electrode 306 with a dielectric (insulating) layer interposed therebetween. The first dielectric layer 310 separates the working electrode 304 and the reference electrode, or separates the counter electrode 306 and the reference electrode 312, from each other to provide electrical isolation.

In one or more aspects, the reference electrode 312 can be a Ag/AgCl electrode.

In one or more aspects, the second dielectric layer 314 can be on the reference electrode 312, and the metal-containing layer 316 can be on the second dielectric layer 314 and electrochemically communicate with the working electrode 304. In some aspects, different from the configuration shown in FIGS. 3A and 3B, the second dielectric layer 314 and the metal-containing layer 316 can be on the side of the counter electrode 306 with the second dielectric layer 314 interposed therebetween. The second dielectric layer 314 separates the working electrode 304 and the metal-containing layer 316, or separates the counter electrode 306 and the antimicrobial metal-containing layer 316, from each other so that the metal-containing layer 316 does not directly contact the working electrode 306. In some aspects, the metal-containing layer 316 can be located any suitable place on the implantable portion 300, and a dielectric layer interposes between the metal-containing layer 316 and the working electrode 304, aspects of the present disclosure are not limited thereto.

The membrane 318 overcoats at least sensing layer 308. In one or more aspects, the membrane 318 can overcoat some or all of the working electrode 304, the counter electrode 306, and the reference 312, or the entirety of the implantable portion 300 of the analyte sensor. One or both faces of the implantable portion 300 of the analyte sensor can be overcoated with the membrane 318. The membrane 318 can include one or more polymeric membrane materials, such as a polymer matrix, having capabilities of limiting analyte flux to the sensing layer 308 (i.e., the membrane 318 is a diffusion-limiting membrane having some permeability for the analyte of interest). In one or more aspects, the membrane 318 can be crosslinked with a branched crosslinker in certain particular sensor configurations. The composition and thickness of the membrane 318 can vary to promote a desired or suitable analyte flux to the sensing layer 308, thereby providing a desired or suitable signal intensity and stability. The detailed description of the membrane 318 can refer to the membrane disclosed herein. The analyte sensor can be operable for assaying an analyte by any of coulometric, amperometric, voltammetric, or potentiometric electrochemical detection techniques.

One or more aspects of the present disclosure are directed toward a method of actively releasing an antimicrobial agent for an analyte sensor, the method including:
inserting an analyte sensor described herein and comprising at least one antimicrobial agent and/or a hydrogel coating that includes a metal-containing layer in electrochemical communication with the counter electrode, the reference electrode, a second working electrode, or any combination thereof into a tissue (e.g., skin) of a patient; and
electrochemically generating an antimicrobial agent by applying electric current to the analyte sensor.

One or more aspects of the present disclosure are directed to an analyte sensor for detecting an analyte in vivo, the sensor comprising:
a proximal portion configured to be positioned above a user's skin; and
a distal portion configured to be transcutaneously positioned beneath the skin and in contact with a bodily fluid to detect the analyte in vivo.

In one or more aspects, the distal portion comprises:
a working electrode,
a sensing layer disposed on at least a portion of the working electrode that comprises an analyte-responsive enzyme, and
a polymer membrane overcoating at least the sensing layer,
wherein the polymer membrane comprises an antimicrobial agent disposed therein, a hydrogel coating disposed thereon, or both an antimicrobial agent disposed therein and hydrogel coating disposed thereon.

One or more aspects of the present disclosure are directed to a sensor control device comprising:
(i) a processor; and
(ii) an analyte sensor as disclosed herein, wherein the sensor obtains a signal indicative of the analyte concentration in a bodily fluid and communicates the signal indicative of the analyte concentration to the processor.

One or more aspects of the present disclosure are directed to an analyte sensing system comprising:
(i) an analyte sensor as disclosed herein; and
(ii) a processor configured to (a) correlate a signal indicative of analyte concentration obtained by the sensor to analyte concentration in a bodily fluid;
and to (b) communicate the analyte concentration to a reader device to be displayed.

One or more aspects of the present disclosure are directed to a method comprising:
exposing the analyte sensor as disclosed herein to a bodily fluid;
obtaining a signal at or above an oxidation-reduction potential of the active area, the signal being proportional to a concentration of the analyte in a bodily fluid contacting the sensing layer; and
correlating the signal to the analyte concentration in the bodily fluid

In one or more aspects, the metal-containing layer can include: (i) a metal selected from the group consisting of silver, copper, zinc, gold, platinum, palladium, titanium, an alloy of any of the foregoing, and mixtures of any of the foregoing; (ii) and optionally a corresponding metal salt thereof.

In one or more aspects, the metal can be in a form of a film, a wire, or a particle, as described herein. In one or more aspects, the metal-containing layer can include: (i) a silver film, silver wires, and/or silver particles; and (ii) a silver salt. In some aspects, the silver salt can be silver chloride. In one or more aspects, the metal-containing layer can be a screen-printed silver coating including silver particles and a silver salt.

In one or more aspects, the antimicrobial agent can be electrochemically generated metal ions such as silver ions, zinc ions, copper ions, gold ions, platinum ions, palladium ions, titanium ions, etc. In some aspects, the electrochemically generated antimicrobial agent is electrochemically generated silver ions.

In an aspect, electrochemically generating an antimicrobial agent by applying an anodic electric current to the analyte sensor comprises applying a potential suitable to oxidize the metal in the metal-containing layer. In one or more aspects, e.g., when the metal-containing layer comprises silver (Ag) optionally in combination with silver chloride (AgCl) and the reference electrode comprises Ag/AgCl, the step of electrochemically generating an antimicrobial agent by applying electric current to the analyte sensor can include applying a potential between +30 mV and +200 mV between the metal-containing layer and the reference electrode for a set or predetermined or set duration by utilizing a potentiostat. The applied potential can be between +40 mV and +200 mV, +50 mV and +200 mV, +60 mV and +200 mV, +70 mV and +200 mV, +80 mV and +200 mV, +90 mV and +200 mV, +100 mV and +200 mV, +110 mV and +200 mV, +120 mV and +200 mV, +130 mV and +200 mV, +140 mV and +200 mV, +150 mV and +200 mV, +160 mV and +200 mV, +170 mV and +200 mV, +180 mV and +200 mV, +190 mV and +200 mV, or any ranges in between, such as between +150 mV and +190 mV.

In one or more aspects, the antimicrobial agent can be electrochemically generated metal ions such as silver ions, zinc ions, copper ions, gold ions, platinum ions, palladium ions, titanium ions, etc. In some aspects, the electrochemically generated antimicrobial agent can be electrochemically generated silver ions.

In one or more aspects, electrochemically generating an antimicrobial agent by applying electric current to the analyte sensor can include applying a predetermined or set current between the metal-containing layer and the counter electrode by utilizing a galvanostat.

In one or more aspects, electrochemically generating an antimicrobial agent by applying electric current to the analyte sensor can include applying a predetermined or set current between the metal-containing layer and the counter electrode for a predetermined or set duration by utilizing a galvanostat.

In one or more aspects, the antimicrobial agent can be galvanostatically/potentiostatically generated at a basal rate of metal ion generation.

In one or more aspects, the antimicrobial agent can be galvanostatically/potentiostatically generated in a set or predetermined amount for a set or predetermined period release such as for one-day release, two-day release, three-day release, etc.

In one or more aspects, the metal-containing layer such as a silver-containing layer can have a thickness in a range of about 0.1 μm to about 1,000 μm, e.g., from about 1 μm to and about 500 μm, about 1 μm to about 300 μm, or about 1 μm to about 100 μm.

In one or more aspects, the analyte sensor can be configured to detect glucose.

In one or more aspects, the analyte sensor can be a dermal sensor.

In one or more aspects, the analyte sensor can be a subcutaneous analyte sensor such as a subcutaneously implanted biosensor.

In one or more aspects, the analyte sensor can be an intravenous sensor such as intravenously implanted sensor.

One or more aspects of the present disclosure are directed toward a method of manufacturing an analyte sensor as described herein, the method including: applying a polymer solution comprising a polymer to at least a portion of the implantable portion to form the polymer membrane and optionally the hydrogel coating.

In one or more aspects, the method can further comprise curing the polymer membrane and/or the hydrogel coating.

In one or more aspects, curing the polymer membrane and/or the hydrogel coating can comprise curing the polymer membrane and/or the hydrogel coating for less than 15 days, for example, for less than 1 day, about 1 day, about 2 days, about 3 days, about 4 days, about 5 days, about 6 days, about 7 days, about 8 days, about 9 days, about 10 days, about 11 days, about 12 days, about 13 days, or about 14 days, or any ranges in between two aforementioned values, such as about 5 to about 6 days. In one or more aspects curing the polymer membrane and/or the hydrogel coating can comprise curing the polymer membrane and/or the hydrogel coating for about 2 to about 3 days.

In one or more aspects, applying the polymer solution to at least a portion of the implantable portion can comprise dipping the implantable portion into a polymer solution to coat at least a portion of the implantable portion to form the polymer membrane and/or hydrogel coating. In one or more aspects, applying a polymer solution to at least a portion of the implantable portion can include spraying the polymer solution to at least a portion of the implantable portion to form the polymer membrane and/or hydrogel coating. In another aspect, the antimicrobial agent (e.g., one or more antibiotics) can be incorporated within a sprayed or dipped membrane and can be spatially located within the polymer membrane. For example, the antimicrobial agent can be present only in a single layer (e.g., only in the outside layer, only in a middle/interior layer, or only on the inner (initial) dip layer) or in any combination of layers (e.g., present in the outer, middle/interior, and inner (initial) dip layers or present only in the outer and middle/interior layers).

In one or more aspects, the polymer solution can further comprise a crosslinker to form the polymer membrane and/or hydrogel coating.

In one or more aspects, the polymer solution can further comprise a solvent, such as a solvent in which the polymer and/or antimicrobial agent(s) are soluble. In one or more aspects, the solvent can be water, an alcohol (e.g., ethanol, methanol, propanol, isopropanol), acetone, ethyl acetate, acetonitrile, dichloromethane, toluene, or any combination thereof. In one or more aspects, the solvent can comprise one or more of water, ethanol, methanol, and acetone.

In one or more aspects, an amount of the antimicrobial agent in the polymer membrane can be in a range of about 0.1 wt % to about 40 wt % based on a total weight of the polymer (e.g., dry mass) and the antimicrobial agent. In one or more aspects, an amount of the antimicrobial agent in the membrane can be in a range of about 5 wt % to about 15 wt % based on a total weight of the polymer (e.g., dry mass) and the antimicrobial agent. In one or more aspects, an amount of the antimicrobial agent in the membrane can be in a range of about 10 wt % to about 15 wt % based on a total weight of the polymer (e.g., dry mass) and the antimicrobial.

In one or more aspects, the polymer solution can have a viscosity in a range of about 50 cP to about 250 cP, for example, about 60 cP to about 250 cP, about 70 cP to about 250 cP, about 80 cP to about 250 cP, about 90 cP to about 250 cP, about 100 cP to about 250 cP, about 100 cP to about 240 cP, about 100 cP to about 230 cP, about 100 cP to about 220 cP, about 100 cP to about 210 cP, or about 100 cP to about 200 cP, or any values in between. In some aspects, a viscosity of the polymer solution can be in a range of about 50 cP to about 120 cP, about 60 cP to about 130 cP, about 70 cP to about 140 cP, about 80 cP to about 150 cP, about 90 cP to about 160 cP, about 100 cP to about 170 cP, about 110 cP to about 180 cP, about 120 cP to about 190 cP, or about 130 cP to about 200 cP. However, aspects of the present disclosure are not limited thereto.

In one or more aspects, polymer solution can comprise about 1 mg/mL to about 200 mg/mL polymer per ml of solution, for example, about 1 mg/mL to about 180 mg/mL, about 1 mg/mL to about 160 mg/mL, about 1 mg/mL to about 140 mg/mL, about 1 mg/mL to about 120 mg/mL, about 1 mg/mL to about 100 mg/mL, about 1 mg/mL to about 90 mg/mL, about 1 mg/mL to about 80 mg/mL, about 1 mg/mL to about 70 mg/mL, about 1 mg/mL to about 60 mg/mL, about 1 mg/mL to about 50 mg/mL, about 5 mg/mL to about 150 mg/mL, about 10 mg/mL to about 150 mg/mL, about 20 mg/mL to about 150 mg/mL, about 30 mg/mL to about 150 mg/mL, about 40 mg/mL to about 150 mg/mL, about 50 mg/mL to about 150 mg/mL, or about 60 mg/mL to about 150 mg/mL, or any values in between. In some aspects, the amount of polymer included in the polymer solution can be in a range of about 1 mg/mL to about 20 mg/mL, about 20 mg/mL to about 40 mg/mL, about 40 mg/mL to about 60 mg/mL, about 60 mg/mL to about 80 mg/mL, about 80 mg/mL to about 100 mg/mL, about 90 mg/mL to about 110 mg/mL, about 100 mg/mL to about 120 mg/mL, or about 120 mg/mL to about 140 mg/mL.

In one or more aspects, dipping the implantable portion into the polymer solution to coat at least a portion of the implantable portion to form the polymer membrane and/or hydrogel coating can comprise dipping the implantable portion into the polymer solution to coat at least a portion of the implantable portion at a dipping speed in a range of 0.01 mm/s to 10 mm/s, for example, 0.1 mm/s to 10 mm/s, 0.1 mm/s to 9 mm/s, 0.1 mm/s to 8 mm/s, 0.1 mm/s to 7 mm/s, 0.1 mm/s to 6 mm/s, 0.1 mm/s to 5 mm/s, 0.1 mm/s to 4 mm/s, 0.1 mm/s to 3 mm/s, 0.1 mm/s to 2 mm/s, or 0.1 mm/s to 1 mm/s.

### EXAMPLES

The example presented below is provided for the purpose of illustration only and the aspects described herein should in no way be construed as being limited to these examples. Rather, the aspects should be construed to encompass any and all variations which become evident as a result of the teaching provided herein.

### Example 1

Antibiotics minocycline hydrochloride and rifampin were loaded into a polyvinylpyridine-co-polystyrene polymer (e.g., 10Q5-01) membrane (using the procedure that follows) to extend sensor in vivo functionality through reduced effects associated with the presence of microbes within an insertion site.

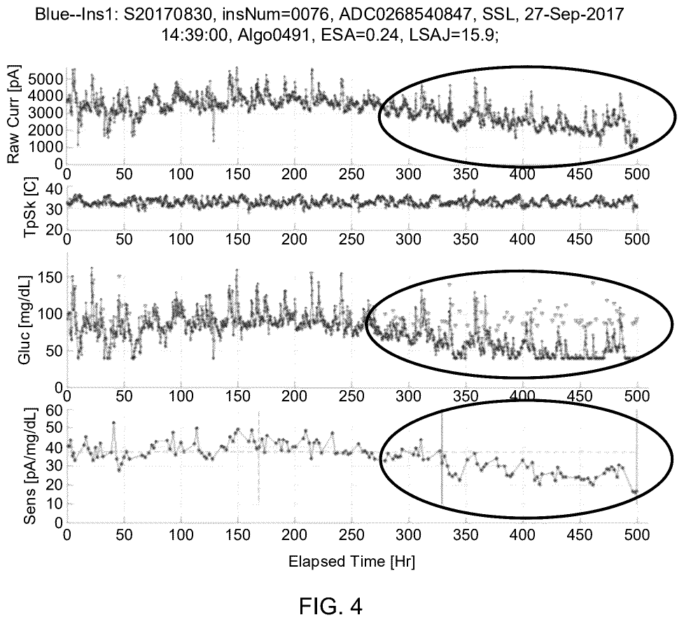

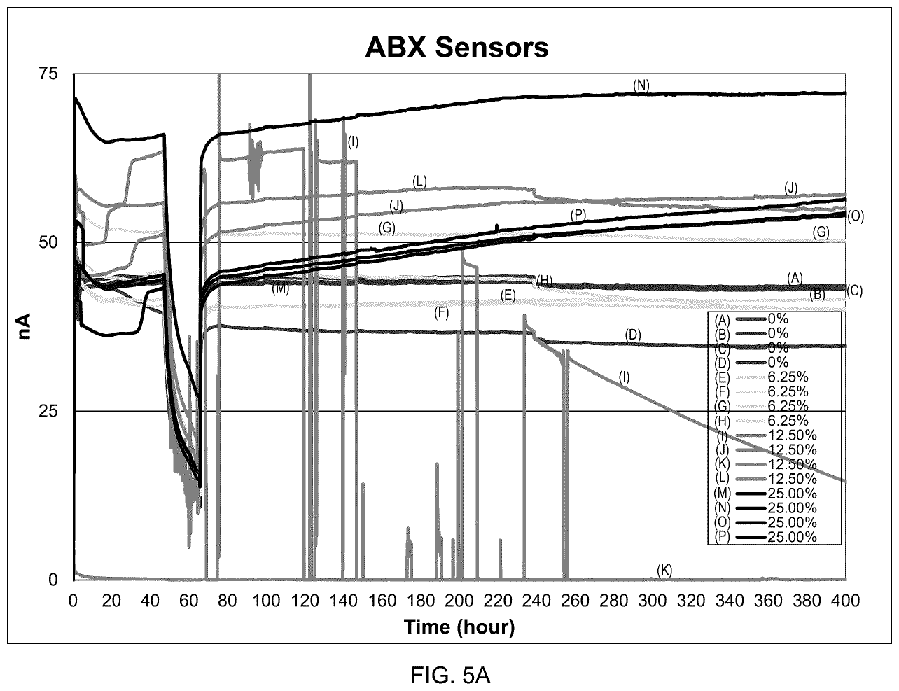

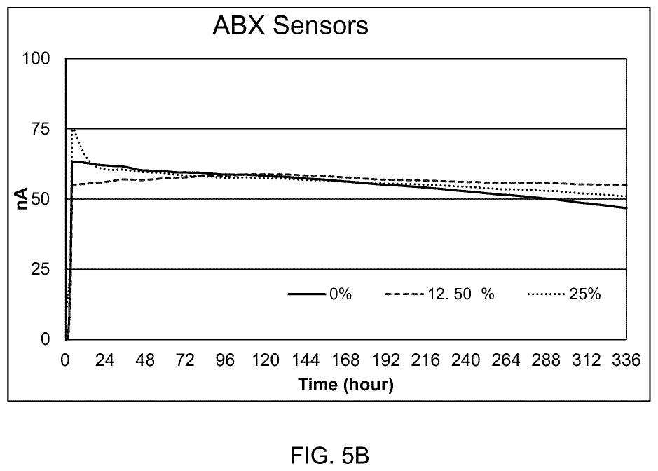

Microorganism infection at the implant site can lead to biofilm formation directly on the implant surface. Biofouling from protein and other molecule adsorption onto sensor surfaces can produce an increasing and limiting diffusion barrier between a sensor and an analyte, resulting in artificially low analyte readings. For example, Bacilli (rod-shaped) and Cocci (round) colony-forming units (CFUs) have been observed on the surface of an analyte sensor according to one or more aspects of the present disclosure. FIG. 4 illustrates late signal attenuation of an analyte sensor caused by bacteria CFUs according to one or more aspects of the present disclosure. Late signal attenuation is identifiable by a dynamically dropping (circled portion in FIG. 4) in sensitivity/reduced functionality or accuracy deviation. The analyte sensor studied in FIG. 5 is a glucose sensor and trends may not be representative of other analyte sensors that may exhibit different patterns of accuracy reduction (such as go up or reduced/increased dynamics). In some aspects, patterns can also be cyclic or inconsistent.

To reduce the late signal attenuation associated with the presence of microbes at or in an insertion site and/or on the surface of the analyte sensor, minocycline hydrochloride and rifampin were incorporated into a polyvinylpyridine-co-polystyrene polymer (10Q5-01) membrane, as the diffusion limiting polymer membrane, of glucose sensors.

A 10Q5-01 membrane dipping formulation typically contains ethanol, but a direct volumetric substitution with methanol provided an increased loading of both antibiotics into the dipping polymer solution.

FIGS. 5A and 5B illustrate responses of analyte sensors made with methanol-based dipping solutions and various wt % antibiotic loadings. The wt % values were based on total dry membrane weight and 1:1 by weight of minocycline hydrocholoride and rifampin. Stability at 30 mM glucose in 100 mM PBS is shown (results were duplicated).

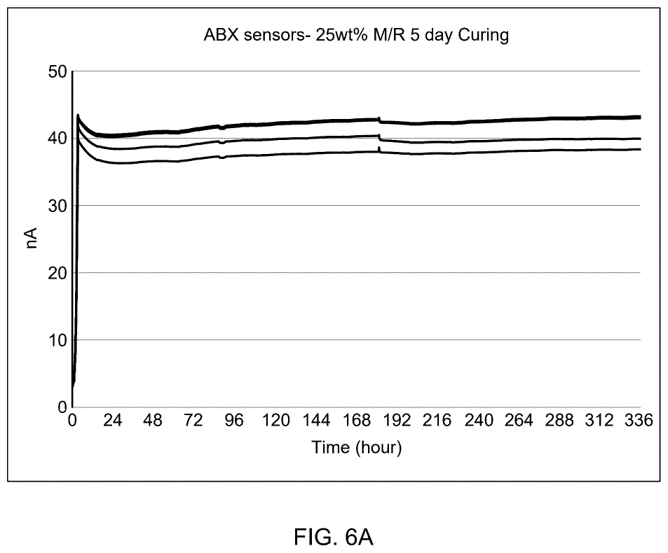

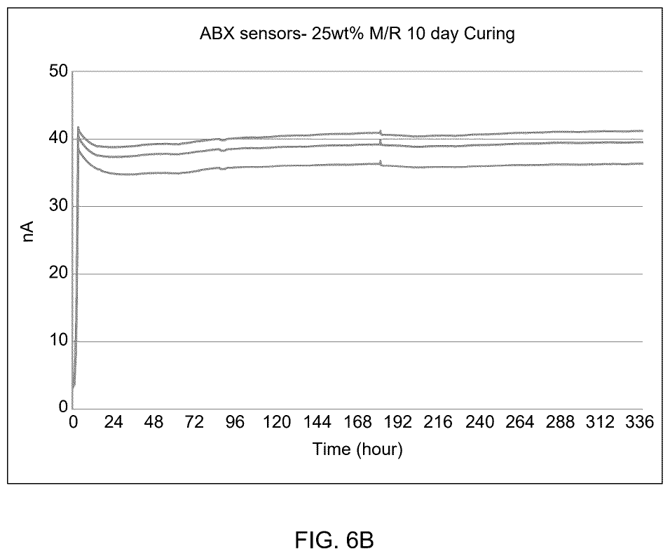

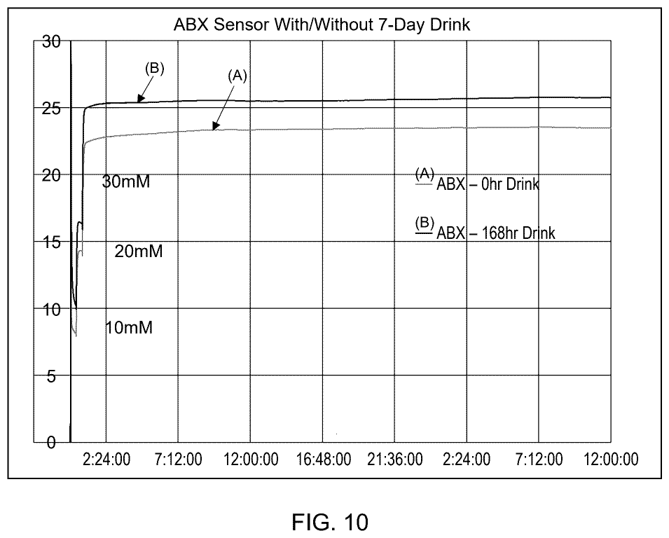

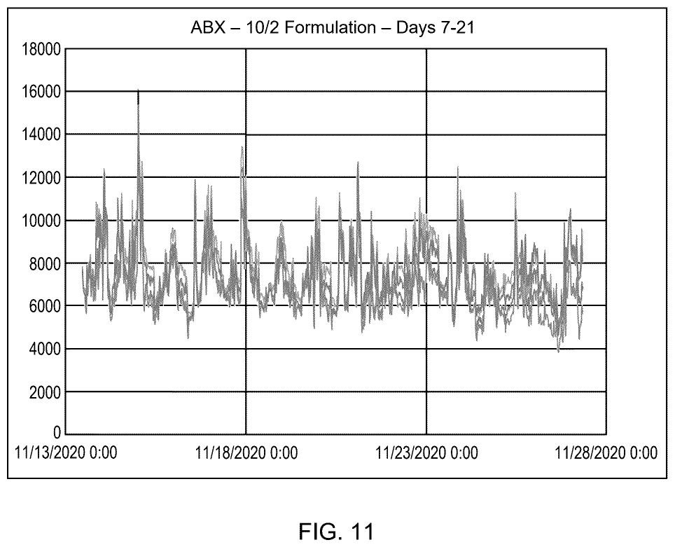

FIGS. 6A and 6B illustrate responses of antimicrobial sensors made with methanol-based dipping solutions, 25 wt % antibiotic loadings, and 5-days curing (FIG. 6A) or 10-days curing (FIG. 6B). FIGS. 5A and 5B (24-hour curing time and 25 wt % loading) show a drop of about 25% during the first 24 hours, whereas 5 day and 10 day curing times showed a drop of only 10% in the first 24 hours.

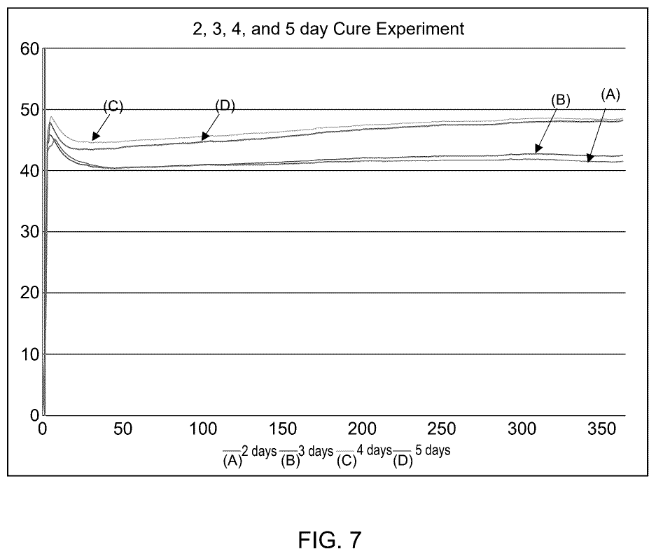

FIG. 7 illustrates responses of antimicrobial sensors made with methanol-based dipping solutions, 25 wt % antibiotic loadings, and different curing times. FIG. 6A and FIG. 6B show an improved 24 hour run for 5 and 10 days curing times versus 24 hours curing time. FIG. 7 showed a curing time of 2 to 3 days provided improved stability compared to longer and shorter curing times.

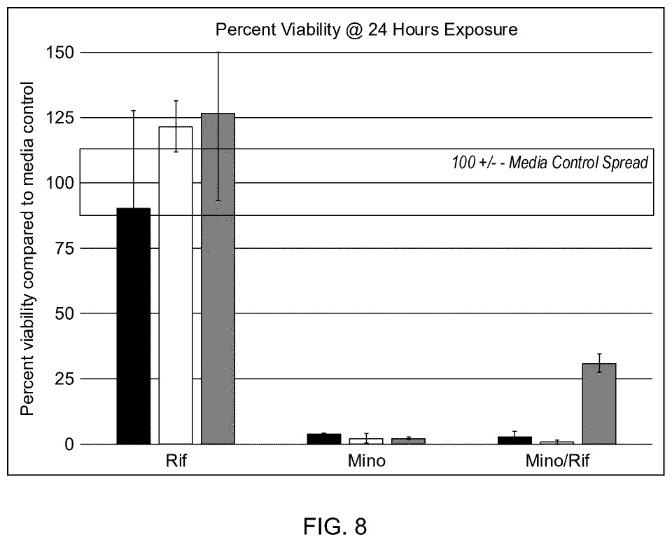

Although in vitro beaker testing indicated the 25 wt % dipping formulation, i.e., including 12.5 wt % of rifampin and 12.5 wt % of minocycline hydrochloride, could stabilized by replacing ethanol with methanol and increasing the curing time to 2 or 3 days, cytotoxicity testing indicated a potential biocompatibility concern associated with using 12.5 wt % of minocycline hydrochloride. FIG. 8 illustrates cytotoxicity testing of antimicrobial sensors. Coupons representative of (i) 25 wt % formulation sensors and (ii) each antibiotic was extracted into cell culture media. Media were tested at neat (blue; left bar in each series), 50% dilution (orange; middle bar in each series), and 75% dilution (gray; right bar in each series) for rifampin (“Rif”), minocycline hydrochloride (“Mino”), and a combination of minocycline hydrochloride and rifampin (“Mino/Rif”). As shown in FIG. 8, cytotoxicity testing of coupon extracts suggested potential toxicity concerns with 12.5 wt % minocycline hydrochloride loading.

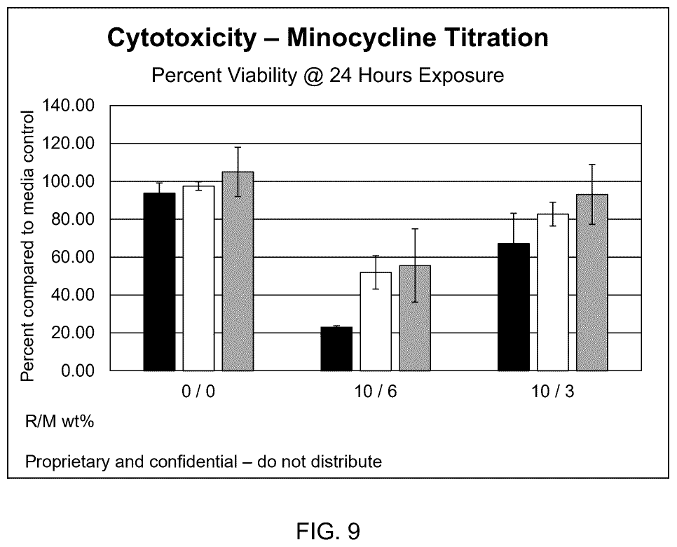

Thus, minocycline hydrochloride concentration was titrated for acceptable toxicity in the presence of 10 wt % rifampin. FIG. 9 illustrates cytotoxicity testing of antimicrobial sensors including different minocycline hydrochloride loadings. Coupons representative of (i) 0 (no antibiotics), (ii) 16 wt % (10 wt % rifampin/6 wt % minocycline hydrochloride), and (iii) 13 wt % (10 wt % rifampin/3 wt % minocycline hydrochloride) formulation sensors were extracted into cell culture media. Media were tested at neat (black; left bar in each series) and 50% dilution (white; middle bar in each series) and 75% dilution (gray; right bar in each series) for no antibiotics (“0/0”), 16 total weight % (“10/6:”), and 13 total wt % (“10/3”). As shown in FIG. 9, cytotoxicity testing of coupon extracts indicated reduced toxicity with a reduced minocycline hydrochloride amount. For example, reduced cytotoxicity was observed for 3 wt % and 6 wt % minocycline hydrochloride based on the total weight of the polymer matrix (e.g., dry mass) and antibiotic.

FIG. 10 illustrates sensor calibration and 36-hour stability testing of antimicrobial sensors. Glucose sensors including a polymer membrane containing 10Q5-01 (polyvinylpyridine-co-polystyrene polymer), 10 wt % rifampin, and 2 wt % minocycline hydrochloride were assessed for in vitro robustness. Sensors were exposed to an infinite PBS drink at 37° C. with agitation for 168 hours (preconditioned to stimular an exaggerate wear scenario). Sensor calibration with a suitable glucose concentration (e.g., 10 mM, 20, mM, or 30 mM) and 36 hour stability testing of sensors as produced and post-drink exposure indicated that the sensors were highly stable (FIG. 10).

### Example 2

Antibacterial efficacy of antimicrobial sensors was further studied after three weeks in vivo wear.

The tested bacteria lines included Staphylococcus epidermidis (S. epi), Staphylococcus aureus subsp. aureus strain (UAMS-1), methicillin-resistant Staphylococcus aureus (MIRSA), Enterococcus faecalis (E. faecalis), Cutibacterium acnes (C. acnes), and Streptococcus pyogenes (S. pyogenes).

Nineteen tryptic soy agar (TSA) plates were used (5 each for S. epi & UAMS-1, 9 forMRSA). Nine brain heart infusion agar plates were used for E. faecalis. Eighteen blood agar plates were used for C. acnes and S. pyogenes (9 each).

Analyte sensors with a polymer membrane comprising 10Q5-01 polymer (polyvinylpyridine-co-polystyrene polymer), 10 wt % rifampin, and 2 wt % minocycline hydrochloride were prepared. Sterile phosphate-buffered saline (PBS) and tryptic soy broth (TSB) were used. Three sensors were placed in each tube in PBS (3 mL per sensor) while agitating at 37° C. For S. epi, UAMS-1, MRSA, C. acnes, and S. pyogenes, 50 μl of each bacteria was pipetted into separate 5 mL tubes with 2 mL TSB. For E. faecalis: 50 μl of E. faecalis was pipetted into a 5 mL tube with 2 mL brain heart infusion broth (BHIB). Each 5 mL tube was vortexed and placed into an incubator for overnight growth.

Under aseptic conditions, the PBS was drained out of the tubes that have the three sensors. Each sensor was dipped into sterile PBS to clean them and the sensors were placed onto sterile gauze to dry. Each sensor was flipped over to fully dry both sides.

One hundred (100) μl of bacteria was placed onto the corresponding plates. Rolling beads were used to spread the bacteria onto the plates. A cloth reference point was placed in the middle of the plate. The dried sensors from each group were placed on to the corresponding plates with adequate space surrounding each sensor. The plates were placed into an incubator for overnight growth.

For each of the samples, the antimicrobial sensors inhibited the growth and colony formation of the bacteria (i.e., Staphylococcus epidermidis, Staphylococcus aureus subsp. aureus strain (UAMS-1), Enterococcus faecalis, Staphylococcus aureus (MIRSA), Cutibacterium acnes, and Streptococcus pyogenes) around the sensors, especially around the implantable portions, for at least 21 days.

### Example 3

Bacteriostatic efficacy of antimicrobial sensors was further studied after at least three week in vivo wear (i.e., post-mortem testing).

The antimicrobial activity of analyte sensors with a polymer membrane comprising 10Q5-01 polymer (polyvinylpyridine-co-polystyrene polymer), 10 wt % rifampin, and 2 wt % minocycline hydrochloride was measured after three weeks of in vivo wear according to one or more aspects of the present disclosure. Sustained antimicrobial activity out to at least three weeks in vivo wear was observed. After three weeks of in vivo wear, even though the bacteriostatic properties of the antibiotic sensor (ABX, 21 days) were reduced compared to the as-produced sensor (ABX, 0 days), the antibiotic activity of the polymer membrane of the sensor was still maintained around the implantable portion of the sensor. A Libre™ sensor (Abbott Diabetes Care, Alameda, CA) negative control sensor (Libre™, 0 days) exhibited no inhibition of the colony formation of bacteria.

A comparison of sensors after three weeks of in vivo wear was made for: an antimicrobial (ABX) sensor with a polymer membrane comprising 10Q5-01 polymer (polyvinylpyridine-co-polystyrene polymer), 10 wt % rifampin, and 2 wt % minocycline hydrochloride at both 0 days (initial) and 21 days (post mortem) and a Libre™ sensor (Abbott Diabetes Care, Alameda, CA) (control) sensor at 0 days. It was observed that the red color of the rifampin was seen in the antibiotic sensors (ABX) and not in the Libre™ control. The post mortem ABX sensor had a reduced intensity but still prominent red color, which indicated that rifampin was still present in the polymer membrane after 21 days of wear.

### Example 4

The late signal attenuation characteristics of an antimicrobial sensor were evaluated.

FIG. 11 illustrates example response characteristics of 4 antimicrobial sensors according to one or more aspects of the present disclosure. The antimicrobial sensors were coated with a polymer membrane produced utilizing a 10 wt % rifampin/2 wt % minocycline hydrocholoride/10Q5-01 formulation. As shown in FIG. 11, during the last 14 days of a 21 day wear study, all four antimicrobial sensor exhibited little or no late signal attenuation.

### Example 5

To reduce the early signal attenuation, a hydrogel coating for an analyte sensor was investigated.

An implantable portion comprising a crosslinked 10Q5 diffusion limiting polymer membrane was overcoated with a hydrogel coating. The dipping formulation for the hydrogel coating is shown in Table 1.

TABLE 1

| | Final mg/mL | |
|---|---|---|
| Hydrogel Solution | PAA | PAA + NaF |
| Polyacrylic acid (molecular weight: 100 kDa) | 72 | 72 |
| NaF (mg) | — | 42 |
| trimethylolpropane tris(2-methyl- | 3 | 3 |
| 1-aziridinepropionate | | |

| | |
|---|---|
| Dipping Condition (mm/sec) | 1 × 10 |

A hydrogel coating was coated on the analyte sensor by a dipping method with a dipping speed of 10 mm/s. The Libre™ sensor had a weight of 15.94 mg, and the weight of the hydrogel coating was about 0.052 mg (dry mass).

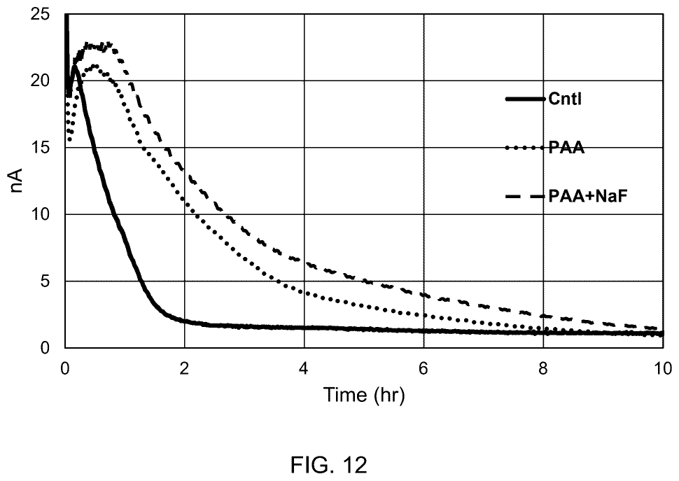

FIG. 12 illustrates responses of analyte sensors in non-heparinized blood in a silicon tube at 37° C. according to one or more aspects of the present disclosure. As shown in FIG. 12, the analyte sensors coated with poly(acrylic acid) hydrogel (dotted line) or poly(acrylic acid)/sodium fluoride hydrogel (dashed line) had improved signal responses compared with the analyte sensor without a hydrogel coating (solid line). Thus, the presence of the hydrogel coating reduced the early signal attenuation.

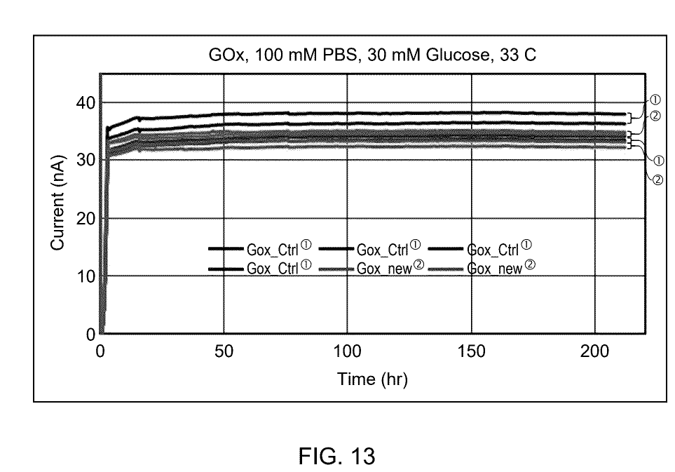

FIG. 13 illustrates responses of analyte sensors in a 30 mM glucose and 100 mM PBS solution at 33° C. As shown in FIG. 13, the analyte sensors coated with poly(acrylic acid) hydrogel (red; bottom lines) had improved signal responses in an extended testing period compared with an analyte sensor control without a hydrogel coating (black; including upper two lines). Thus, the presence of the hydrogel coating provided an analyte sensor exhibiting both reduced early signal attenuation and late signal attenuation.

### Example 6

To reduce the late signal attenuation, an analyte sensor with a metal-containing layer in electrochemical communication with the reference electrode and/or the counter electrode was investigated.

On the implantable portion of a transcutaneous analyte sensor, a silver ink (no AgCl) was screen-printed onto the area above a reference electrode on the front side and/or the area above a counter electrode on the backside, to form a silver ink coating. A carbon contact pad was printed and provided (e.g., a 4th carbon contact pad in a dual sensor design) on a flag part of the implantable portion, and carbon traces (reference carbon and counter carbon) were printed and provided to connect to the printed silver ink coating.

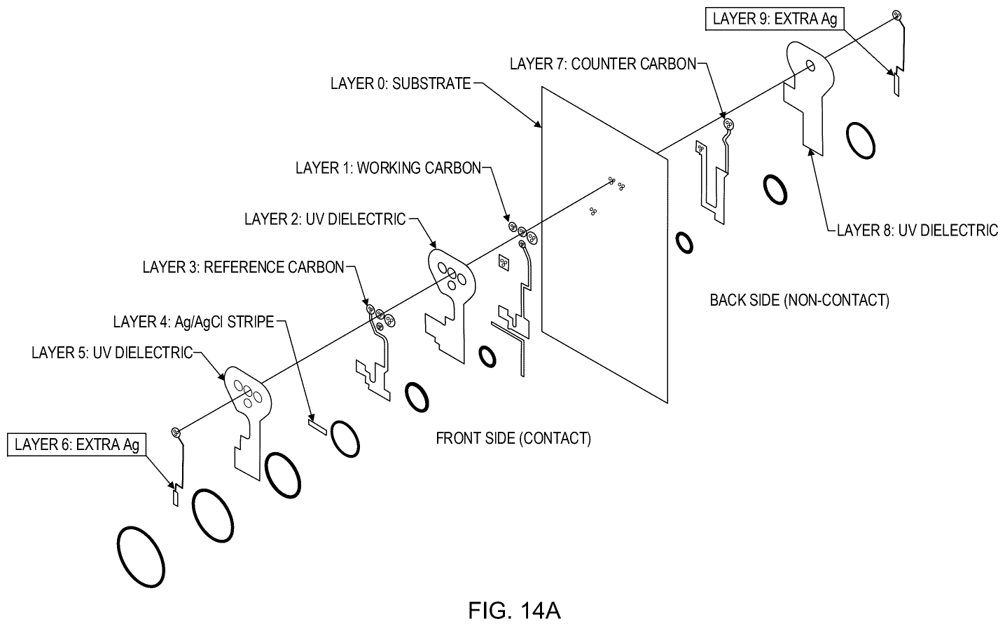

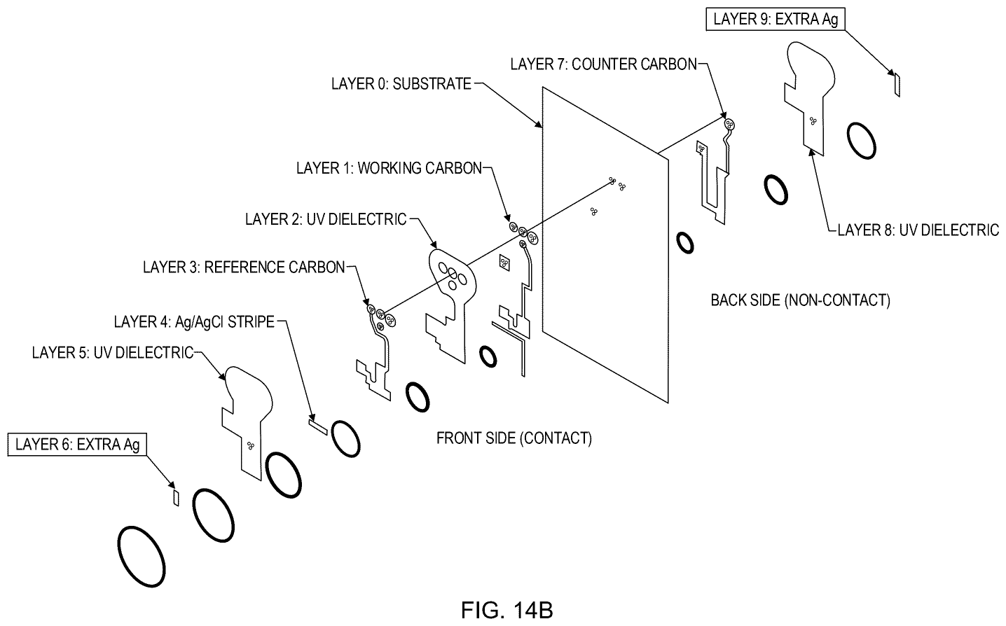

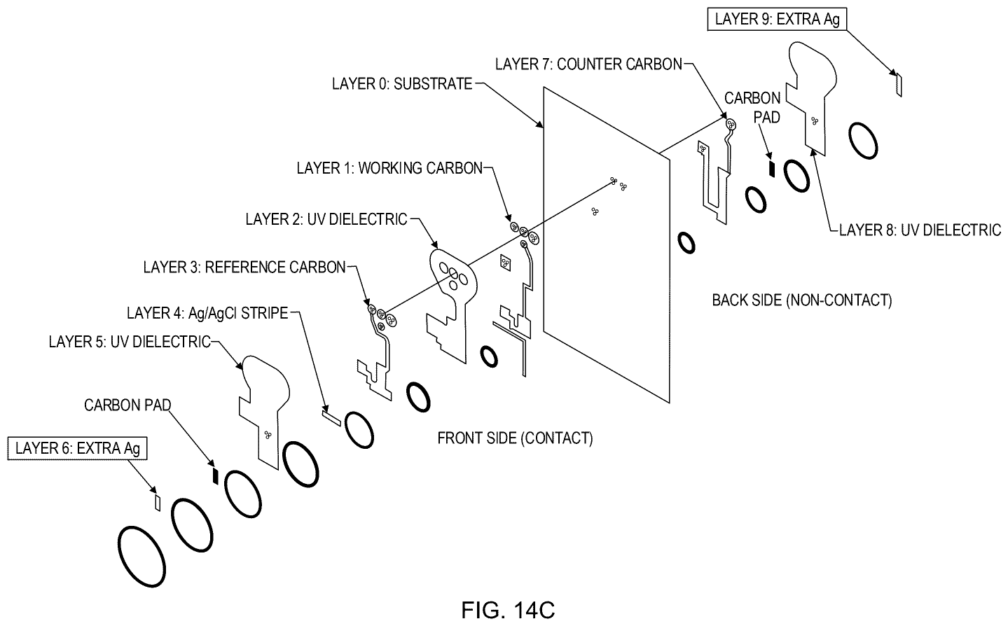

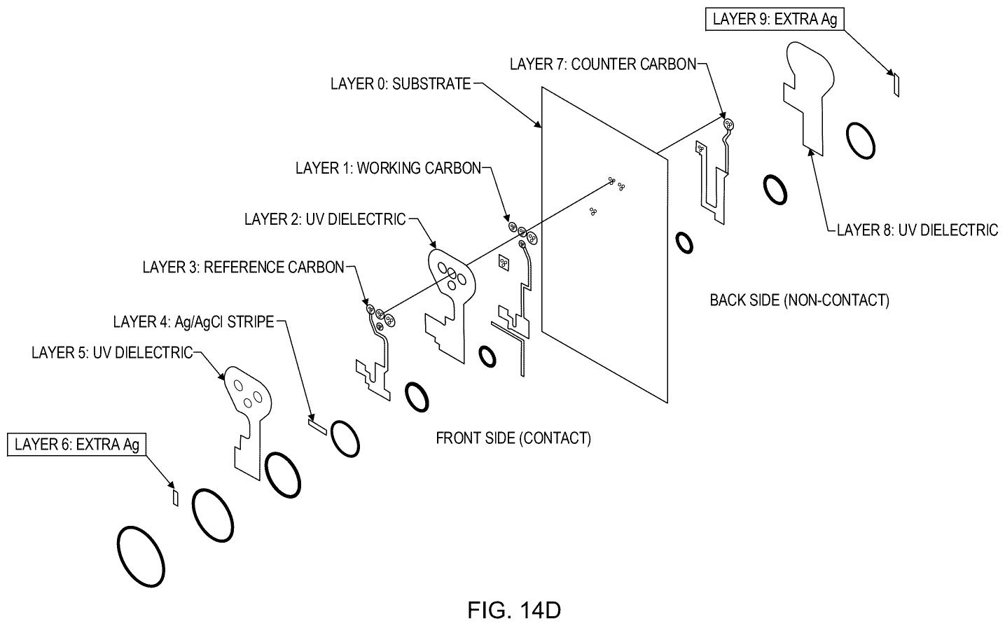

As shown in FIG. 14A, the analyte sensor can include a substrate (Layer 0) having a front side (contact) (a first side) and a back side (non-contact) (a second side) opposite to the front side; a working carbon layer (layer 1) on the front side of the substrate; a first UV dielectric layer (layer 2) on the working carbon layer; a reference carbon layer (layer 3) on the UV dielectric layer; an Ag/AgCl stripe (layer 4) on the reference carbon layer; a second UV dielectric layer (layer 5) on the Ag/AgCl stripe; a first extra Ag layer (i.e., a first Ag layer, layer 6) on the second UV dielectric layer that is in electrical communication with the reference carbon (layer 3); a counter carbon layer (layer 7) on the second side of the substrate; a third UV dielectric layer (layer 8) on the counter carbon layer; and a second extra Ag layer (i.e., a second Ag layer, layer 9) on the third UV dielectric layer that is in electrical communication with the counter carbon (layer 7). The first extra Ag layer and/or second extra Ag layer are in electrochemical communication with the Ag/AgCl stripe, which acts as a reference electrode, and the counter carbon layer, which functions as a counter electrode.

As shown in FIG. 14B, the analyte sensor can include a substrate (Layer 0) having a front side (contact) (a first side) and a back side (non-contact) (a second side) opposite to the front side; a working carbon layer (layer 1) on the front side of the substrate; a first UV dielectric layer (layer 2) on the working carbon layer; a reference carbon layer (layer 3) on the UV dielectric layer; an Ag/AgCl stripe (layer 4) on the reference carbon layer; a second UV dielectric layer (layer 5) on the Ag/AgCl stripe; a first extra Ag layer (i.e., a first Ag layer, layer 6) on the second UV dielectric layer that is in electrical communication with the reference carbon (layer 3) thru the vias in the UV dielectric layer (layer 5); a counter carbon layer (layer 7) on the second side of the substrate; a third UV dielectric layer (layer 8) on the counter carbon layer; and a second extra Ag layer (i.e., a second Ag layer, layer 9) on the third UV dielectric layer that is in electrical communication with the counter carbon (layer 7) thru the vias in the UV dielectric layer (layer 8). The first extra Ag layer and/or second extra Ag layer are in electrochemical communication with the Ag/AgCl stripe, which acts as a reference electrode, and the counter carbon layer, which functions as a counter electrode.

As shown in FIG. 14C, the analyte sensor can include a substrate (Layer 0) having a front side (contact) (a first side) and a back side (non-contact) (a second side) opposite to the front side; a working carbon layer (layer 1) on the front side of the substrate; a first UV dielectric layer (layer 2) on the working carbon layer; a reference carbon layer (layer 3) on the UV dielectric layer; an Ag/AgCl stripe (layer 4) on the reference carbon layer; a second UV dielectric layer (layer 5) on the Ag/AgCl stripe; a first extra Ag layer (i.e., a first Ag layer, layer 6) on the second UV dielectric layer that is in electrical communication with the reference carbon (layer 3) thru the vias in the UV dielectric layer (layer 5) and the carbon pad that can optionally fill the vias; a counter carbon layer (layer 7) on the second side of the substrate; a third UV dielectric layer (layer 8) on the counter carbon layer; and a second extra Ag layer (i.e., a second Ag layer, layer 9) on the third UV dielectric layer that is in electrical communication with the counter carbon (layer 7) thru the vias in the UV dielectric layer (layer 8) and the carbon pad that can optionally fill the vias. The first extra Ag layer and/or second extra Ag layer are in electrochemical communication with the Ag/AgCl stripe, which acts as a reference electrode, and the counter carbon layer, which functions as a counter electrode.

When generating Ag ions, at least one of the first extra Ag layer or the second extra Ag layer electrochemically communicates with the reference and counter electrodes via a bi-potentiostat, such as utilizing a dual-sensor design configuration. In an aspect, the bi-potentiostat can apply a potential between +30 mV and +200 mV relative to the reference electrode to actively generate silver ions. The applied duration can be adjusted as needed.

Alternatively, with a galvanostat, a controlled or selected current mode can be used to inject a positive current into the first extra Ag layer and/or the second extra Ag layer (i.e., printed silver ink layers) to generate Ag ions. As such, the precise amount of Ag ions can be generated by controlling the current level and duration.

There are many possible operation methods to generate the antimicrobial Ag ions. For example, a basal rate of Ag ion generation can be turned on galvanostatically; a bolus charge can also be injected as needed; a certain amount of AgCl enough for release in one day can be generated potentiostatically by turning on the electrochemical communication between the Ag layer and the reference/counter electrodes daily, and so on.

AgCl can be generated electrochemically in site on electrodes. U.S. Pat. Nos. 8,280,474 and 9,042,955, the disclosures of each of which are incorporated herein in their entirety, disclose methods to extend the lifetime of an amperometric analyte sensor by extending the screen-printed Ag/AgCl reference electrode lifetime.

In some aspects, FIG. 14D shows an analyte sensor with a substrate (Layer 0) having a front side (contact) (a first side) and a back side (non-contact) (a second side) opposite to the front side; a working carbon layer (layer 1) on the front side of the substrate; a first UV dielectric layer (layer 2) on the working carbon layer; a reference carbon layer (layer 3) on the UV dielectric layer; an Ag/AgCl stripe (layer 4) on the reference carbon layer; a second UV dielectric layer (layer 5) on the Ag/AgCl stripe; a first extra Ag layer (i.e., a first Ag layer, layer 6) on the second UV dielectric layer that is in not in electrical communication with the reference carbon (layer 3); a counter carbon layer (layer 7) on the second side of the substrate; a third UV dielectric layer (layer 8) on the counter carbon layer; and a second extra Ag layer (i.e., a second Ag layer, layer 9) on the third UV dielectric layer that is not in electrical communication with the counter carbon (layer 7). The first extra Ag layer and second extra Ag layer are electrically isolated and can passively dissolve in the presence of water (e.g., during in vivo use) to release antimicrobial silver and/or silver chloride.

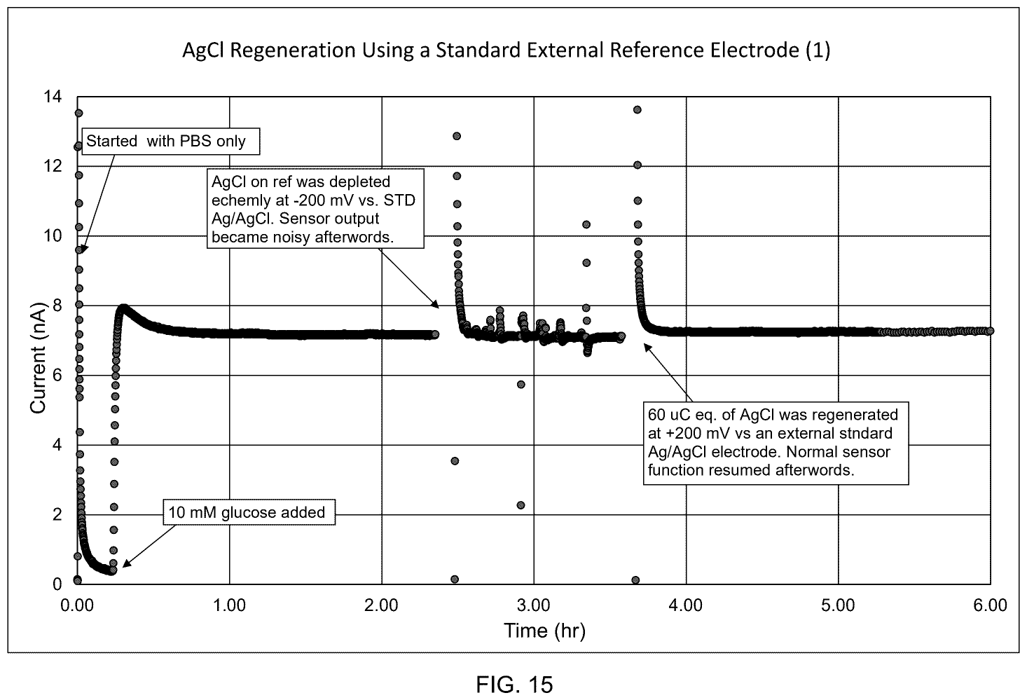

For an amperometric analyte sensor, the response signal of the analyte sensor can become unstable after use in a long term. Without wishing to be bound by any theory, it is believed to be the life of Ag/AgCl reference electrodes being reduced by the loss of AgCl through its slow dissolution into the aqueous solution such as the bodily fluid. FIG. 15 illustrates an electrochemical generation of AgCl according to one or more aspects of the present disclosure. As shown in FIG. 15, after AgCl on the reference electrode of the analyte sensor was depleted electrochemically at −200 mV vs. an external standard Ag/AgCl reference electrode, the sensor output became noisy. Once all the AgCl of the reference electrode of the analyte sensor was released, the potential of the reference electrode became unstable, which caused the noisy sensor signal. By applying a +200 mV vs. the external standard Ag/AgCl reference electrode, a certain amount of AgCl was regenerated, and normal sensor function was resumed. The AgCl was generated by electrochemically oxidizing the Ag metal to form Ag ions which subsequently were precipitated by chloride ions as AgCl. Therefore, the antimicrobial Ag ions can be actively electrochemically generated by oxidizing the Ag metal in a controlled and selective mode for antimicrobial applications.

The present disclosure also provides the following numbered aspects.

Aspect 1. An analyte sensor comprising
a working electrode,
a sensing layer disposed on at least a portion of the working electrode that comprises an analyte-responsive enzyme, and
a polymer membrane overcoating at least the sensing layer,
wherein the polymer membrane comprises an antimicrobial agent disposed therein, a hydrogel coating disposed thereon, or both an antimicrobial agent disposed therein and hydrogel coating disposed thereon.

Aspect 2. An analyte sensor for measuring an analyte concentration in a bodily fluid of a user, the analyte sensor comprising:
a proximal portion positionable above a surface of a skin and electrically coupled with a processor configured to (a) correlate a signal indicative of analyte concentration obtained by the sensor to analyte concentration in the bodily fluid; and to (b) communicate the analyte concentration to a reader device to be displayed,
a distal portion positionable below the surface of the skin, wherein the distal portion is in contact with bodily fluid and configured to monitor analyte concentration in the bodily fluid,
the distal portion comprising a working electrode, a reference electrode, and a counter electrode, each connected to contacts positioned on the proximal portion, wherein the working electrode comprises at least one sensing layer;
wherein the sensing layer comprises:
   an analyte-responsive enzyme; and
   a polymer membrane overcoating at least the sensing layer,
wherein the polymer membrane comprises an antimicrobial agent disposed therein, a hydrogel coating disposed thereon, or both an antimicrobial agent disposed therein and hydrogel coating disposed thereon.

Aspect 3. The analyte sensor of aspect 1 or 2, wherein the polymer membrane comprises a polymer matrix formed from at least one polymer selected from the group consisting of a polyvinylpyridine-based polymer, a polyvinylimidazole-based polymer, a polyacrylate, a poly(amino acid), a polyurethane, a polyether urethane, a silicone, and any combination thereof.

Aspect 4. The analyte sensor of aspect 3, wherein the polymer matrix is formed from a polyvinylpyridine-based polymer, a polyvinylimidazole-based polymer, or a combination thereof.

Aspect 5. The analyte sensor of aspect 3 or 4, wherein the polyvinylpyridine-based polymer is an optionally substituted polyvinylpyridine-co-polystyrene polymer.

Aspect 6. The analyte sensor of any one of aspects 3-5, wherein the polymer matrix is further formed from a crosslinker.

Aspect 7. The analyte sensor of aspect 6, wherein the crosslinker is selected from the group consisting of polyepoxides, carbodiimide, cyanuric chloride, triglycidyl glycerol, N-hydroxysuccinimide, imidoesters, epichlorohydrin, amine-containing compounds, and any combination thereof.

Aspect 8. The analyte sensor of aspect 7, wherein the crosslinker is a diglycidyl- or triglycidyl-functional epoxy.

Aspect 9. The analyte sensor of aspect 8, wherein the crosslinker is selected from the group consisting of diglycidyl-PEG 200, diglycidyl-PEG 400, diglycidyl-PEG 1000, glycerol triglycidyl ether, and any combination thereof.

Aspect 10. The analyte sensor of any one of aspects 1-9, wherein the polymer membrane comprises an antimicrobial agent.

Aspect 11. The analyte sensor of aspect 10, wherein the antimicrobial agent is an antibiotic, an anti-fungal agent, an anti-infective agent, or any combination thereof and is not a metal or a metal salt.

Aspect 12. The analyte sensor of aspect 10 or 11, wherein the antimicrobial agent comprises at least one antibiotic.

Aspect 13. The analyte sensor of aspect 12, wherein the antibiotic comprises a tetracycline class antibiotic.

Aspect 14. The analyte sensor of aspect 13, wherein the tetracycline class antibiotic is minocycline or a salt thereof.

Aspect 15. The analyte sensor of aspect 14, wherein the minocycline salt is minocycline hydrochloride.

Aspect 16. The analyte sensor of aspect 15, wherein the minocycline hydrochloride is added to the polymer matrix in an amount of about 0.1 to about 5 wt %.

Aspect 17. The analyte sensor of any one of aspects 12-16, wherein the antibiotic comprises the tetracycline class antibiotic in combination with an ansamycin class antibiotic.

Aspect 18. The analyte sensor of aspect 17, wherein the ansamycin class antibiotic is a rifamycin.

Aspect 19. The analyte sensor of aspect 18, wherein the rifamycin is rifampin.

Aspect 20. The analyte sensor of aspect 19, wherein the antibiotic comprises minocycline hydrochloride and rifampin in a weight ratio of ranging from about 1:10 to about 10:1.

Aspect 21. The analyte sensor of aspect 20, wherein the weight ratio of minocycline hydrochloride to rifampin ranges from about 1:6 to about 1:4.

Aspect 22. The analyte sensor of any one of aspects 12-21, wherein a total amount of the antibiotic is in a range of about 0.1 wt % to about 40 wt % based on a total weight of the polymer matrix.

Aspect 23. The analyte sensor of aspect 22, wherein the total amount of the antibiotic is in a range of about 5 wt % to about 20 wt % based on a total weight of the polymer matrix.

Aspect 24. The analyte sensor of any one of aspects 1-24, wherein the polymer membrane comprises a hydrogel coating overcoating the polymer membrane.

Aspect 25. The analyte sensor of aspect 24, wherein the hydrogel coating is formed from a polymer selected from poly(acrylic acid), poly-(α,β)-DL-aspartic acid, poly-L-glutamic acid, a salt form thereof, and a combination thereof, and a crosslinker.

Aspect 26. The analyte sensor of aspect 25, wherein the polymer is poly(acrylic acid), a salt form thereof, or a combination thereof.

Aspect 27. The analyte sensor of aspect 25 or 26, wherein the crosslinker comprises trimethylolpropane tris(2-methyl-1-aziridinepropionate).

Aspect 28. The analyte sensor of any one of aspects 24-27, wherein the hydrogel coating further comprises a fluoride ion compound.

Aspect 29. The analyte sensor of aspect 28, wherein the fluoride ion compound is at least one selected from an alkali metal fluoride, an alkaline earth metal fluoride, a transition metal fluoride, an ammonium fluoride, and any combination thereof.

Aspect 30. The analyte sensor of aspect 28 or 29, wherein the fluoride ion compound is sodium fluoride.

Aspect 31. The analyte sensor of any one of aspects 28-30, wherein a total amount of the fluoride ion is in a range of about 1 wt % to about 30 wt % based on a total weight of the hydrogel coating.

Aspect 32. The analyte sensor of any one of aspects 1-31, wherein the analyte sensor further comprises a metal-containing layer in electrochemical communication with the reference electrode, the counter electrode, a second working electrode, or any combination thereof.

Aspect 33. The analyte sensor of aspect 32, wherein the metal-containing layer comprises: (i) a metal selected from the group consisting of silver, copper, zinc, gold, platinum, palladium, titanium, an alloy of any of the foregoing, and mixtures of any of the foregoing; and (ii) optionally a corresponding metal salt thereof.

Aspect 34. The analyte sensor of aspect 32 or 33, wherein the metal is in a form of a film, a wire, or particles.

Aspect 35. The analyte sensor of any one of aspects 32-34, wherein the metal-containing layer comprises: (i) a silver film, silver wire, and/or silver particles; and (ii) a silver salt.

Aspect 36. The analyte sensor of aspect 35, wherein the silver salt is silver chloride.

Aspect 37. A method of actively releasing an antimicrobial agent in an analyte sensor, the method comprising:
inserting the analyte sensor of any one of claims 32-36 into skin of a patient; wherein the hydrogel coating is in electrochemical communication with the counter electrode and/or the reference electrode; and
electrochemically generating an antimicrobial agent by applying an electric current to the analyte sensor.

Aspect 38. The method of aspect 37, wherein the antimicrobial agent is electrochemically generated metal ions.

Aspect 39. The method of aspect 37 or 38, wherein the antimicrobial agent is electrochemically generated silver ions.

Aspect 40. The method of any one of aspects 37-39, wherein electrochemically generating an antimicrobial agent by applying an anodic electric current to the analyte sensor comprises applying a potential to oxidize the metal in the metal-containing layer.

Aspect 41. An analyte sensor for detecting an analyte in vivo, the sensor comprising:
a proximal portion configured to be positioned above a user's skin; and
a distal portion configured to be transcutaneously positioned beneath the skin and in contact with bodily fluid to detect the analyte in vivo;
the distal portion comprising:
   a working electrode,
   a sensing layer disposed on at least a portion of the working electrode that comprises an analyte-responsive enzyme, and
   a polymer membrane overcoating at least the sensing layer,
   wherein the polymer membrane comprises an antimicrobial agent disposed therein, a hydrogel coating disposed thereon, or both an antimicrobial agent disposed therein and hydrogel coating disposed thereon.

Aspect 42. The analyte sensor of aspect 41, wherein the polymer membrane comprises a polymer matrix formed from at least one polymer selected from the group consisting of a polyvinylpyridine-based polymer, a polyvinylimidazole-based polymer, a polyacrylate, a poly(amino acid), a polyurethane, a polyether urethane, a silicone, and any combination thereof.

Aspect 43. The analyte sensor of aspect 42, wherein the polymer matrix is formed from a polyvinylpyridine-based polymer, a polyvinylimidazole-based polymer, or a combination thereof.

Aspect 44. The analyte sensor of aspect 42 or 43, wherein the polyvinylpyridine-based polymer is an optionally substituted polyvinylpyridine-co-polystyrene polymer.

Aspect 45. The analyte sensor of any one of aspects 42-44, wherein the polymer matrix is further formed from a crosslinker.

Aspect 46. The analyte sensor of aspect 45, wherein the crosslinker is selected from the group consisting of polyepoxides, carbodiimide, cyanuric chloride, triglycidyl glycerol, N-hydroxysuccinimide, imidoesters, epichlorohydrin, amine-containing compounds, and any combination thereof.

Aspect 47. The analyte sensor of aspect 46, wherein the crosslinker is a diglycidyl- or triglycidyl-functional epoxy.

Aspect 48. The analyte sensor of aspect 47, wherein the crosslinker is selected from the group consisting of diglycidyl-PEG 200, diglycidyl-PEG 400, diglycidyl-PEG 1000, glycerol triglycidyl ether, and any combination thereof.

Aspect 49. The analyte sensor of any one of aspects 41-448, wherein the polymer membrane comprises an antimicrobial agent.

Aspect 50. The analyte sensor of aspect 49, wherein the antimicrobial agent is an antibiotic, an anti-fungal agent, an anti-infective agent, or any combination thereof and is not a metal or a metal salt.

Aspect 51. The analyte sensor of aspect 49 or 50, wherein the antimicrobial agent comprises at least one antibiotic.

Aspect 52. The analyte sensor of aspect 51, wherein the antibiotic comprises a tetracycline class antibiotic.

Aspect 53. The analyte sensor of aspect 52, wherein the tetracycline class antibiotic is minocycline or a salt thereof.

Aspect 54. The analyte sensor of aspect 53, wherein the minocycline salt is minocycline hydrochloride.

Aspect 55. The analyte sensor of aspect 54, wherein the minocycline hydrochloride is added to the polymer matrix in an amount of about 0.1 to about 5 wt %.

Aspect 56. The analyte sensor of any one of aspects 51-55, wherein the antibiotic comprises the tetracycline class antibiotic in combination with an ansamycin class antibiotic.

Aspect 57. The analyte sensor of aspect 56, wherein the ansamycin class antibiotic is a rifamycin.

Aspect 58. The analyte sensor of aspect 57, wherein the rifamycin is rifampin.

Aspect 59. The analyte sensor of aspect 58, wherein the antibiotic comprises minocycline hydrochloride and rifampin in a weight ratio of ranging from about 1:10 to about 10:1.

Aspect 60. The analyte sensor of aspect 59, wherein the weight ratio of minocycline hydrochloride to rifampin ranges from about 1:6 to about 1:4.

Aspect 61. The analyte sensor of any one of aspects 51-60, wherein a total amount of the antibiotic is in a range of about 0.1 wt % to about 40 wt % based on a total weight of the polymer matrix.

Aspect 62. The analyte sensor of aspect 61, wherein the total amount of the antibiotic is in a range of about 5 wt % to about 20 wt % based on a total weight of the polymer matrix.

Aspect 63. The analyte sensor of any one of aspects 41-63, wherein the polymer membrane comprises a hydrogel coating overcoating the polymer membrane.

Aspect 64. The analyte sensor of aspect 63, wherein the hydrogel coating is formed from a polymer selected from poly(acrylic acid), poly-(α,β)-DL-aspartic acid, poly-L-glutamic acid, a salt form thereof, and a combination thereof, and a crosslinker.

Aspect 65. The analyte sensor of aspect 64, wherein the polymer is poly(acrylic acid), a salt form thereof, or a combination thereof.

Aspect 66. The analyte sensor of aspect 64 or 65, wherein the crosslinker comprises trimethylolpropane tris(2-methyl-1-aziridinepropionate).

Aspect 67. The analyte sensor of any one of aspects 63-66, wherein the hydrogel coating further comprises a fluoride ion compound.

Aspect 68. The analyte sensor of aspect 67, wherein the fluoride ion compound is at least one selected from an alkali metal fluoride, an alkaline earth metal fluoride, a transition metal fluoride, an ammonium fluoride, and any combination thereof.

Aspect 69. The analyte sensor of aspect 67 or 68, wherein the fluoride ion compound is sodium fluoride.

Aspect 70. The analyte sensor of any one of aspects 67-69, wherein a total amount of the fluoride ion is in a range of about 1 wt % to about 30 wt % based on a total weight of the hydrogel coating.

Aspect 71. The analyte sensor of any one of aspects 41-70, wherein the analyte sensor further comprises a metal-containing layer in electrochemical communication with the reference electrode, the counter electrode, a second working electrode, or any combination thereof.

Aspect 72. The analyte sensor of aspect 71, wherein the metal-containing layer comprises: (i) a metal selected from the group consisting of silver, copper, zinc, gold, platinum, palladium, titanium, an alloy of any of the foregoing, and mixtures of any of the foregoing; and (ii) optionally a corresponding metal salt thereof.

Aspect 73. The analyte sensor of aspect 71 or 72, wherein the metal is in a form of a film, a wire, or particles.

Aspect 74. The analyte sensor of any one of aspects 71-73, wherein the metal-containing layer comprises: (i) a silver film, silver wire, and/or silver particles; and (ii) a silver salt.

Aspect 75. The analyte sensor of aspect 74, wherein the silver salt is silver chloride.

Aspect 76. A sensor control device comprising:
(i) a processor; and
(ii) the analyte sensor of any one of aspects 41-75, wherein the sensor obtains a signal indicative of the analyte concentration in the bodily fluid and communicates the signal indicative of the analyte concentration to the processor.

Aspect 77. An analyte sensing system comprising:
(iii) the analyte sensor of any one of aspects 41-75; and
(iv) a processor configured to (a) correlate a signal indicative of analyte concentration obtained by the sensor to analyte concentration in the bodily fluid; and to (b) communicate the analyte concentration to a reader device to be displayed.

Aspect 78. A method comprising:
exposing the analyte sensor of any one of aspects 41-75 to the bodily fluid;
obtaining a signal at or above an oxidation-reduction potential of the active area, the signal being proportional to a concentration of the analyte in the bodily fluid contacting the sensing layer; and
correlating the signal to the analyte concentration in the bodily fluid.

It is to be appreciated that the Detailed Description section, and not the Summary and Abstract sections, is intended to be used to interpret the claims. The Summary and Abstract sections may set forth one or more but not all exemplary aspects of the present invention as contemplated by the inventor(s), and thus, are not intended to limit the present invention and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the invention that others can, by applying knowledge within the skill of the art, readily modify and/or adapt for various applications such specific aspects, without undue experimentation, without departing from the general concept of the present invention. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary aspects, but should be defined only in accordance with the following claims and their equivalents.

The claims in the instant application are different than those of the parent application or other related applications. The Applicant therefore rescinds any disclaimer of claim scope made in the parent application or any predecessor application in relation to the instant application. The Examiner is therefore advised that any such previous disclaimer and the cited references that it was made to avoid, may need to be revisited. Further, the Examiner is also reminded that any disclaimer made in the instant application should not be read into or against the parent application.

1. An analyte sensor for detecting an analyte in vivo, the sensor comprising
a proximal portion configured to be positioned above a user's skin; and
a distal portion configured to be transcutaneously positioned beneath the skin and in contact with bodily fluid to detect the analyte in vivo;
the distal portion comprising:
a working electrode,
a sensing layer disposed on at least a portion of the working electrode that comprises an analyte-responsive enzyme, and
a polymer membrane overcoating at least the sensing layer,
wherein the polymer membrane comprises an antimicrobial agent disposed therein, a hydrogel coating disposed thereon, or both an antimicrobial agent disposed therein and hydrogel coating disposed thereon.

2. (canceled)

3. The analyte sensor of claim 1, wherein the polymer membrane comprises a polymer matrix formed from at least one polymer selected from the group consisting of a polyvinylpyridine-based polymer, a polyvinylimidazole-based polymer, a polyacrylate, a poly(amino acid), a polyurethane, a polyether urethane, a silicone, and any combination thereof.

4. (canceled)

5. The analyte sensor of claim 3, wherein the polyvinylpyridine-based polymer is an optionally substituted polyvinylpyridine-co-polystyrene polymer.

6. The analyte sensor of claim 3, wherein the polymer matrix is further formed from a crosslinker selected from the group consisting of polyepoxides, carbodiimide, cyanuric chloride, triglycidyl glycerol, N-hydroxysuccinimide, imidoesters, epichlorohydrin, amine-containing compounds, and any combination thereof.

7. (canceled)

8. (canceled)

9. The analyte sensor of claim 6, wherein the crosslinker is selected from the group consisting of diglycidyl-PEG 200, diglycidyl-PEG 400, diglycidyl-PEG 1000, glycerol triglycidyl ether, and any combination thereof.

10. The analyte sensor of claim 1, wherein the polymer membrane comprises an antimicrobial agent.

11. The analyte sensor of claim 10, wherein the antimicrobial agent is an antibiotic, an anti-fungal agent, an anti-infective agent, or any combination thereof and is not a metal or a metal salt.

12. (canceled)

13. (canceled)

14. The analyte sensor of claim 11, wherein the antibiotic is minocycline or a salt thereof.

15. The analyte sensor of claim 14, wherein the minocycline salt is minocycline hydrochloride, and wherein the minocycline hydrochloride is added to the polymer matrix in an amount of about 0.1 to about 5 wt %.

16. (canceled)

17. The analyte sensor of claim 11, wherein the antibiotic comprises a tetracycline class antibiotic in combination with an ansamycin class antibiotic.

18. (canceled)

19. The analyte sensor of claim 17, wherein the ansamycin class antibiotic is rifampin.

20. The analyte sensor of claim 19, wherein the antibiotic comprises minocycline hydrochloride and rifampin in a weight ratio of ranging from about 1:10 to about 10:1.

21. (canceled)

22. The analyte sensor of claim 11, wherein a total amount of the antibiotic is in a range of about 0.1 wt % to about 40 wt % based on a total weight of the polymer matrix.

23. (canceled)

24. The analyte sensor of claim 1, wherein the polymer membrane comprises a hydrogel coating overcoating the polymer membrane, wherein the hydrogel coating comprises a crosslinker and a polymer selected from the group consisting of poly(acrylic acid), poly-(α,β)-DL-aspartic acid, poly-L-glutamic acid, a salt form thereof, and a combination thereof.

25. (canceled)

26. (canceled)

27. The analyte sensor of claim 24, wherein the crosslinker comprises trimethylolpropane tris(2-methyl-1-aziridinepropionate).

28. The analyte sensor of claim 24, wherein the hydrogel coating further comprises sodium fluoride in a total amount ranging from about 1 wt % to about 30 wt % based on a total weight of the hydrogel coating.

29. (canceled)

30. (canceled)

31. (canceled)

32. The analyte sensor of claim 1, wherein the analyte sensor further comprises a metal-containing layer in electrochemical communication with a reference electrode, a counter electrode, a second working electrode, or any combination thereof, wherein the metal-containing layer comprises: (i) a metal selected from the group consisting of silver, copper, zinc, gold, platinum, palladium, titanium, an alloy of any of the foregoing, and mixtures of any of the foregoing; and (ii) optionally a metal salt of the metal in (i).

33. (canceled)

34. (canceled)

35. The analyte sensor of claim 32, wherein the metal-containing layer comprises: (i) a silver film, silver wire, and/or silver particles; and (ii) a silver salt.

36. (canceled)

37. A method of actively releasing an antimicrobial agent in an analyte sensor, the method comprising:
inserting the analyte sensor of claim 32, into skin of a patient;
wherein the hydrogel coating is in electrochemical communication with the counter electrode and/or the reference electrode; and
electrochemically generating an antimicrobial agent by applying an electric current to the analyte sensor.

38. (canceled)

39. The method of claim 37, wherein the antimicrobial agent is electrochemically generated silver ions.

40. (canceled)
